SEQUENCE LISTING (19 sequences)

SEQ ID NO: 1 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = gm
  modified_base 4: mod_base = OTHER; note = 2'-O-methyladenosine
  modified_base 5: mod_base = gm
  modified_base 6: mod_base = um
  modified_base 7: mod_base = um
  modified_base 8: mod_base = gm
  modified_base 9: mod_base = OTHER; note = 2'-O-methyladenosine
  modified_base 10: mod_base = gm
  modified_base 11: mod_base = um
  modified_base 12: mod_base = OTHER; note = 2'-fluorouridine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 19^20: mod_base = OTHER; note = phosphorothioate linkage
  modified_base 20: mod_base = cm
  modified_base 20^21: mod_base = OTHER; note = phosphorothioate linkage
  modified_base 21: mod_base = OTHER; note = 2'-O-methyladenosine
  source 1..21: mol_type = other RNA
ttgagttgagttcaagtgaca

SEQ ID NO: 2 - RNA, 21 nt, synthetic construct
  modified_base 1: mod_base = um
  modified_base 2: mod_base = gm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = cm
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methyladenosine
  source 1..21: mol_type = other RNA
tgtcacttgaactcaactcaa

SEQ ID NO: 3 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = cm
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-fluorocytidine
  modified_base 11: mod_base = um
  modified_base 12: mod_base = OTHER; note = 2'-fluorouridine
  modified_base 13: mod_base = um
  modified_base 14: mod_base = OTHER; note = 2'-fluoroadenosine
  modified_base 15: mod_base = um
  modified_base 16: mod_base = OTHER; note = 2'-fluorouridine
  modified_base 17: mod_base = gm
  modified_base 18: mod_base = OTHER; note = 2'-fluoroadenosine
  modified_base 18^19: mod_base = OTHER; note = phosphorothioate linkage
  modified_base 19: mod_base = gm
  source 1..19: mol_type = other RNA
ttcaaagcactttattgag

SEQ ID NO: 4 - RNA, 19 nt, synthetic construct
  modified_base 1: mod_base = cm
  modified_base 2: mod_base = um
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-fluoroadenosine
  modified_base 8: mod_base = OTHER; note = 2'-fluoroadenosine
  modified_base 9: mod_base = OTHER; note = 2'-fluoroadenosine
  modified_base 10: mod_base = gm
  modified_base 11: mod_base = um
  modified_base 12: mod_base = gm
  modified_base 13: mod_base = cm
  modified_base 14: mod_base = um
  modified_base 15: mod_base = um
  modified_base 16: mod_base = um
  modified_base 17: mod_base = gm
  modified_base 18: mod_base = OTHER; note = 2'-O-methyladenosine
  modified_base 19: mod_base = OTHER; note = 2'-O-methyladenosine
  source 1..19: mol_type = other RNA
ctcaataaagtgctttgaa

SEQ ID NO: 5 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = cm
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-fluorocytidine
  modified_base 11: mod_base = um
  modified_base 12: mod_base = OTHER; note = 2'-fluorouridine
  modified_base 13: mod_base = um
  modified_base 14: mod_base = OTHER; note = 2'-fluoroadenosine
  modified_base 15: mod_base = um
  modified_base 16: mod_base = OTHER; note = 2'-fluorouridine
  modified_base 17: mod_base = gm
  modified_base 18: mod_base = OTHER; note = 2'-fluoroadenosine
  modified_base 18^19: mod_base = OTHER; note = phosphorothioate linkage
  modified_base 19: mod_base = gm
  source 1..19: mol_type = other RNA
ttcaaagcactttattgag

SEQ ID NO: 6 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = gm
  modified_base 4: mod_base = OTHER; note = 2'-O-methyladenosine
  modified_base 5: mod_base = gm
  modified_base 6: mod_base = um
  modified_base 7: mod_base = um
  modified_base 8: mod_base = gm
  modified_base 9: mod_base = OTHER; note = 2'-O-methyladenosine
  modified_base 10: mod_base = gm
  modified_base 11: mod_base = um
  modified_base 12: mod_base = OTHER; note = 2'-fluorouridine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 17^18: mod_base = OTHER; note = phosphorothioate linkage
  modified_base 18: mod_base = gm
  modified_base 18^19: mod_base = OTHER; note = phosphorothioate linkage
  modified_base 19: mod_base = OTHER; note = 2'-O-methyladenosine
  source 1..19: mol_type = other RNA
ttgagttgagttcaagtga

SEQ ID NO: 7 - RNA, 38 nt, synthetic construct
  modified_base 1: mod_base = um
  modified_base 2: mod_base = cm
  modified_base 3: mod_base = OTHER; note = 2'-O-methyladenosine
  modified_base 4: mod_base = cm
  modified_base 5: mod_base = um
  modified_base 6: mod_base = um
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroadenosine
  modified_base 10: mod_base = cm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = cm
  modified_base 20^21: mod_base = OTHER; note = phosphorothioate linkage
  modified_base 21: mod_base = um
  modified_base 21^22: mod_base = OTHER; note = phosphorothioate linkage
  modified_base 22: mod_base = cm
  modified_base 23: mod_base = OTHER; note = 2'-O-methyladenosine
  modified_base 24: mod_base = OTHER; note = 2'-O-methyladenosine
  modified_base 25: mod_base = um
  modified_base 26: mod_base = OTHER; note = 2'-fluoroadenosine
  modified_base 27: mod_base = OTHER; note = 2'-fluoroadenosine
  modified_base 28: mod_base = OTHER; note = 2'-fluoroadenosine
  modified_base 29: mod_base = gm
  modified_base 30: mod_base = um
  modified_base 31: mod_base = gm
  modified_base 32: mod_base = cm
  modified_base 33: mod_base = um
  modified_base 34: mod_base = um
  modified_base 35: mod_base = um
  modified_base 36: mod_base = gm
  modified_base 37: mod_base = OTHER; note = 2'-O-methyladenosine
  modified_base 38: mod_base = OTHER; note = 2'-O-methyladenosine
  source 1..38: mol_type = other RNA
tcacttgaactcaactcaactcaataaagtgctttgaa

SEQ ID NO: 8 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = cm
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-fluorocytidine
  modified_base 11: mod_base = um
  modified_base 12: mod_base = OTHER; note = 2'-fluorouridine
  modified_base 13: mod_base = um
  modified_base 14: mod_base = OTHER; note = 2'-fluoroadenosine
  modified_base 15: mod_base = um
  modified_base 16: mod_base = OTHER; note = 2'-fluorouridine
  modified_base 17: mod_base = gm
  modified_base 18: mod_base = OTHER; note = 2'-fluoroadenosine
  modified_base 18^19: mod_base = OTHER; note = phosphorothioate linkage
  modified_base 19: mod_base = gm
  source 1..19: mol_type = other RNA
ttcaaagcactttattgag

SEQ ID NO: 9 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = gm
  modified_base 4: mod_base = OTHER; note = 2'-O-methyladenosine
  modified_base 5: mod_base = gm
  modified_base 6: mod_base = um
  modified_base 7: mod_base = um
  modified_base 8: mod_base = gm
  modified_base 9: mod_base = OTHER; note = 2'-O-methyladenosine
  modified_base 10: mod_base = gm
  modified_base 11: mod_base = um
  modified_base 12: mod_base = OTHER; note = 2'-fluorouridine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 17^18: mod_base = OTHER; note = phosphorothioate linkage
  modified_base 18: mod_base = gm
  modified_base 18^19: mod_base = OTHER; note = phosphorothioate linkage
  modified_base 19: mod_base = OTHER; note = 2'-O-methyladenosine
  source 1..19: mol_type = other RNA
ttgagttgagttcaagtga

SEQ ID NO: 10 - RNA, 38 nt, synthetic construct; identical to SEQ ID NO: 7
  modified_base 1: mod_base = um
  modified_base 2: mod_base = cm
  modified_base 3: mod_base = OTHER; note = 2'-O-methyladenosine
  modified_base 4: mod_base = cm
  modified_base 5: mod_base = um
  modified_base 6: mod_base = um
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroadenosine
  modified_base 10: mod_base = cm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = cm
  modified_base 20^21: mod_base = OTHER; note = phosphorothioate linkage
  modified_base 21: mod_base = um
  modified_base 21^22: mod_base = OTHER; note = phosphorothioate linkage
  modified_base 22: mod_base = cm
  modified_base 23: mod_base = OTHER; note = 2'-O-methyladenosine
  modified_base 24: mod_base = OTHER; note = 2'-O-methyladenosine
  modified_base 25: mod_base = um
  modified_base 26: mod_base = OTHER; note = 2'-fluoroadenosine
  modified_base 27: mod_base = OTHER; note = 2'-fluoroadenosine
  modified_base 28: mod_base = OTHER; note = 2'-fluoroadenosine
  modified_base 29: mod_base = gm
  modified_base 30: mod_base = um
  modified_base 31: mod_base = gm
  modified_base 32: mod_base = cm
  modified_base 33: mod_base = um
  modified_base 34: mod_base = um
  modified_base 35: mod_base = um
  modified_base 36: mod_base = gm
  modified_base 37: mod_base = OTHER; note = 2'-O-methyladenosine
  modified_base 38: mod_base = OTHER; note = 2'-O-methyladenosine
  source 1..38: mol_type = other RNA

SEQ ID NO: 11 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = cm
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-fluorocytidine
  modified_base 11: mod_base = um
  modified_base 12: mod_base = OTHER; note = 2'-fluorouridine
  modified_base 13: mod_base = um
  modified_base 14: mod_base = OTHER; note = 2'-fluoroadenosine
  modified_base 15: mod_base = um
  modified_base 16: mod_base = OTHER; note = 2'-fluorouridine
  modified_base 17: mod_base = gm
  modified_base 18: mod_base = OTHER; note = 2'-fluoroadenosine
  modified_base 18^19: mod_base = OTHER; note = phosphorothioate linkage
  modified_base 19: mod_base = gm
  source 1..19: mol_type = other RNA
ttcaaagcactttattgag

SEQ ID NO: 12 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = gm
  modified_base 4: mod_base = OTHER; note = 2'-O-methyladenosine
  modified_base 5: mod_base = gm
  modified_base 6: mod_base = um
  modified_base 7: mod_base = um
  modified_base 8: mod_base = gm
  modified_base 9: mod_base = OTHER; note = 2'-O-methyladenosine
  modified_base 10: mod_base = gm
  modified_base 11: mod_base = um
  modified_base 12: mod_base = OTHER; note = 2'-fluorouridine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 17^18: mod_base = OTHER; note = phosphorothioate linkage
  modified_base 18: mod_base = gm
  modified_base 18^19: mod_base = OTHER; note = phosphorothioate linkage
  modified_base 19: mod_base = OTHER; note = 2'-O-methyladenosine
  source 1..19: mol_type = other RNA
ttgagttgagttcaagtga

SEQ ID NO: 13 - RNA, 38 nt, synthetic construct; identical to SEQ ID NO: 7
  modified_base 1: mod_base = um
  modified_base 2: mod_base = cm
  modified_base 3: mod_base = OTHER; note = 2'-O-methyladenosine
  modified_base 4: mod_base = cm
  modified_base 5: mod_base = um
  modified_base 6: mod_base = um
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroadenosine
  modified_base 10: mod_base = cm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = cm
  modified_base 20^21: mod_base = OTHER; note = phosphorothioate linkage
  modified_base 21: mod_base = um
  modified_base 21^22: mod_base = OTHER; note = phosphorothioate linkage
  modified_base 22: mod_base = cm
  modified_base 23: mod_base = OTHER; note = 2'-O-methyladenosine
  modified_base 24: mod_base = OTHER; note = 2'-O-methyladenosine
  modified_base 25: mod_base = um
  modified_base 26: mod_base = OTHER; note = 2'-fluoroadenosine
  modified_base 27: mod_base = OTHER; note = 2'-fluoroadenosine
  modified_base 28: mod_base = OTHER; note = 2'-fluoroadenosine
  modified_base 29: mod_base = gm
  modified_base 30: mod_base = um
  modified_base 31: mod_base = gm
  modified_base 32: mod_base = cm
  modified_base 33: mod_base = um
  modified_base 34: mod_base = um
  modified_base 35: mod_base = um
  modified_base 36: mod_base = gm
  modified_base 37: mod_base = OTHER; note = 2'-O-methyladenosine
  modified_base 38: mod_base = OTHER; note = 2'-O-methyladenosine
  source 1..38: mol_type = other RNA

SEQ ID NO: 14 - RNA, 19 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 18^19: mod_base = OTHER; note = phosphorothioate linkage
  modified_base 19: mod_base = gm
  source 1..19: mol_type = other RNA
aagttacaaaagcaaaacg

SEQ ID NO: 15 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluorocytidine
  modified_base 5: mod_base = um
  modified_base 6: mod_base = OTHER; note = 2'-fluoroguanosine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-fluoroadenosine
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = cm
  modified_base 18: mod_base = OTHER; note = 2'-fluorocytidine
  modified_base 18^19: mod_base = OTHER; note = phosphorothioate linkage
  modified_base 19: mod_base = cm
  source 1..19: mol_type = other RNA
tcactgagaatactgtccc

SEQ ID NO: 16 - RNA, 38 nt, synthetic construct
  modified_base 1: mod_base = cm
  modified_base 2: mod_base = gm
  modified_base 3: mod_base = um
  modified_base 4: mod_base = um
  modified_base 5: mod_base = um
  modified_base 6: mod_base = um
  modified_base 7: mod_base = OTHER; note = 2'-fluoroguanosine
  modified_base 8: mod_base = cm
  modified_base 9: mod_base = OTHER; note = 2'-fluorouridine
  modified_base 10: mod_base = um
  modified_base 11: mod_base = OTHER; note = 2'-fluorouridine
  modified_base 12: mod_base = um
  modified_base 13: mod_base = gm
  modified_base 14: mod_base = um
  modified_base 15: mod_base = OTHER; note = 2'-O-methyladenosine
  modified_base 16: mod_base = OTHER; note = 2'-O-methyladenosine
  modified_base 17: mod_base = cm
  modified_base 18: mod_base = um
  modified_base 19: mod_base = um
  modified_base 20: mod_base = gm
  modified_base 20^21: mod_base = OTHER; note = phosphorothioate linkage
  modified_base 21: mod_base = gm
  modified_base 22: mod_base = gm
  modified_base 23: mod_base = OTHER; note = 2'-O-methyladenosine
  modified_base 24: mod_base = cm
  modified_base 25: mod_base = OTHER; note = 2'-O-methyladenosine
  modified_base 26: mod_base = OTHER; note = 2'-fluoroguanosine
  modified_base 27: mod_base = OTHER; note = 2'-fluorouridine
  modified_base 28: mod_base = OTHER; note = 2'-fluoroadenosine
  modified_base 29: mod_base = um
  modified_base 30: mod_base = um
  modified_base 31: mod_base = cm
  modified_base 32: mod_base = um
  modified_base 33: mod_base = cm
  modified_base 34: mod_base = OTHER; note = 2'-O-methyladenosine
  modified_base 35: mod_base = gm
  modified_base 36: mod_base = um
  modified_base 37: mod_base = OTHER; note = 2'-O-methylinosine
  modified_base 38: mod_base = OTHER; note = 2'-O-methyladenosine
  source 1..38: mol_type = other RNA
cgttttgcttttgtaacttgggacagtattctcagtna

SEQ ID NO: 17 - RNA, 19 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroadenosine
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluoroguanosine
  modified_base 17: mod_base = um
  modified_base 18: mod_base = OTHER; note = 2'-fluorocytidine
  modified_base 18^19: mod_base = OTHER; note = phosphorothioate linkage
  modified_base 19: mod_base = cm
  source 1..19: mol_type = other RNA
acaaaagcaaaacaggtcc

SEQ ID NO: 18 - RNA, 19 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluorocytidine
  modified_base 5: mod_base = um
  modified_base 6: mod_base = OTHER; note = 2'-fluoroguanosine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-fluoroadenosine
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = cm
  modified_base 18: mod_base = OTHER; note = 2'-fluorocytidine
  modified_base 18^19: mod_base = OTHER; note = phosphorothioate linkage
  modified_base 19: mod_base = cm
  source 1..19: mol_type = other RNA
tcactgagaatactgtccc

SEQ ID NO: 19 - RNA, 38 nt, synthetic construct
  modified_base 1: mod_base = gm
  modified_base 2: mod_base = gm
  modified_base 3: mod_base = OTHER; note = 2'-O-methyladenosine
  modified_base 4: mod_base = cm
  modified_base 5: mod_base = cm
  modified_base 6: mod_base = um
  modified_base 7: mod_base = OTHER; note = 2'-fluoroguanosine
  modified_base 8: mod_base = OTHER; note = 2'-fluorouridine
  modified_base 9: mod_base = OTHER; note = 2'-fluorouridine
  modified_base 10: mod_base = um
  modified_base 11: mod_base = um
  modified_base 12: mod_base = gm
  modified_base 13: mod_base = cm
  modified_base 14: mod_base = um
  modified_base 15: mod_base = um
  modified_base 16: mod_base = um
  modified_base 17: mod_base = um
  modified_base 18: mod_base = gm
  modified_base 19: mod_base = um
  modified_base 20: mod_base = gm
  modified_base 20^21: mod_base = OTHER; note = phosphorothioate linkage
  modified_base 21: mod_base = gm
  modified_base 22: mod_base = gm
  modified_base 23: mod_base = OTHER; note = 2'-O-methyladenosine
  modified_base 24: mod_base = cm
  modified_base 25: mod_base = OTHER; note = 2'-O-methyladenosine
  modified_base 26: mod_base = OTHER; note = 2'-fluoroguanosine
  modified_base 27: mod_base = OTHER; note = 2'-fluorouridine
  modified_base 28: mod_base = OTHER; note = 2'-fluoroadenosine
  modified_base 29: mod_base = um
  modified_base 30: mod_base = um
  modified_base 31: mod_base = cm
  modified_base 32: mod_base = um
  modified_base 33: mod_base = cm
  modified_base 34: mod_base = OTHER; note = 2'-O-methyladenosine
  modified_base 35: mod_base = gm
  modified_base 36: mod_base = um
  modified_base 37: mod_base = OTHER; note = 2'-O-methylinosine
  modified_base 38: mod_base = OTHER; note = 2'-O-methyladenosine
  source 1..38: mol_type = other RNA
ggacctgttttgcttttgtgggacagtattctcagtna

(19) United States
(12) Patent Application Publication
(10) Pub. No.: US 2026/0176621 A1
(43) Pub. Date: Jun. 25, 2026

(54) Metabolically Stabilized Carbohydrate Targeting Ligands For Oligonucleotide Conjugates

(71) Applicant: Arrowhead Pharmaceuticals, Inc., Pasadena, CA (US)

(72) Inventors: Agnieszka Glebocka, Madison, WI (US); Jing Chen, Madison, WI (US); Szymon Klossowski, Middleton, WI (US); Tao Pei, Middleton, WI (US); Jonathan Benson, Stoughton, WI (US); Andrei V. Blokhin, Fitchburg, WI (US); Thomas Endean, Madison, WI (US); Sergey Shuvaev, Verona, WI (US)

(21) Appl. No.: 19/401,558

(22) Filed: Nov. 26, 2025

Related U.S. Application Data
(63) Continuation of application No. PCT/US24/31757, filed on May 30, 2024.
(60) Provisional application No. 63/505,414, filed on May 31, 2023.
(60) Provisional application No. 63/559,728, filed on Feb. 29, 2024.

(51) Int. Cl. C12N 15/11; C12N 15/113

(52) U.S. Cl. CPC C12N 15/111; C12N 15/113; C12N 2310/11; C12N 2310/14

(57) ABSTRACT

The present disclosure relates to delivery platforms that specifically and efficiently direct stabilized RNAi agent payloads to hepatocytes in a subject, in vivo. The delivery platforms disclosed herein include metabolically stabilized N-Acetylgalactosamine (NAG or GalNAc) targeting ligands conjugated to one or more oligonucleotides through a metabolically stabilized linkage that is more stable than a phosphodiester linkage, to facilitate the delivery of the oligonucleotide-based payloads to cells, including to hepatocytes. Pharmaceutical compositions that include the metabolically stabilized RNAi agent conjugate delivery platform are also described, as well as methods of use for the treatment of various diseases and disorders where delivery of a therapeutic payload to a hepatocyte is desirable.

### CROSS REFERENCE TO RELATED APPLICATIONS

This application is a continuation of PCT Application PCT/US24/31757, filed May 30, 2024, which claims the benefit of priority of U.S. Provisional Patent Application Ser. No. 63/505,414, filed on May 31, 2023, and U.S. Provisional Patent Application Ser. No. 63/559,728, filed on Feb. 29, 2024, the contents of each of which are incorporated herein by reference in their entirety.

### SEQUENCE LISTING

This application contains a Sequence Listing which has been submitted in XML format and is hereby incorporated by reference in its entirety. The XML copy is named 30725-WO_SeqListing_2024.05.30.xml, was created on May 30, 2024, and is 255688 bytes in size.

### FIELD OF THE INVENTION

The present disclosure relates to delivery platforms for the delivery of oligonucleotides or oligonucleotide-based agents (e.g., antisense oligonucleotides (ASOs) or double-stranded RNAi agents or small interfering RNAs (siRNAs)), to hepatic cells in vivo, and in particular to hepatocytes. The delivery of RNAi agents using the delivery platforms disclosed herein provide for the inhibition of genes that are expressed in hepatocytes.

### BACKGROUND OF THE INVENTION

Directing therapeutic payloads to specific tissues and cells of interest in a subject in vivo continues to be a great challenge in the field of medicine. This is particularly true for oligonucleotide-based therapeutics, such as antisense oligonucleotides (ASOs) and RNA interference (RNAi) agents (typically comprised of small (or short) interfering RNA that employ chemically modified nucleotides), which have shown great promise and potential to revolutionize the field of medicine and provide potent therapeutic treatment options for previously undruggable diseases; provided, of course, that the therapeutic oligonucleotide can reach the desired cells and tissues in vivo. Indeed, obtaining suitable delivery of oligonucleotide-based therapeutics is and still remains the most pressing challenge to overcome in discovering and identifying viable therapeutics.

While developments over the past few decades have led to a better understanding of how to suitably deliver oligonucleotides to hepatocytes by covalently linking the oligonucleotide payload to a targeting ligand comprised of N-Acetylgalactosamine (NAG or GalNAc), further improvements are needed and desired. Improving delivery can potentially permit less drug to be administered to the patient or subject, which can provide the benefit of reducing the likelihood of toxicological side-effects and potentially lower the costs of the therapeutic as less material will be required to be manufactured.

Once such potential solution is to stabilize the NAG ligand to prevent metabolic disintegration of the ligand. NAG ligands have been shown to degrade under physiological conditions prior to delivering the oligonucleotide payload to hepatocytes.

Thus, there remains a need for a delivery mechanism or platform to specifically and efficiently direct oligonucleotide-based therapeutics, and RNAi agents in particular, to hepatocyte cells.

### SUMMARY

Disclosed herein are delivery platforms that include metabolically stabilized carbohydrate ligands linked to therapeutics, such as oligonucleotide-based therapeutics including antisense oligonucleotides (ASOs) or RNA interference (RNAi) agents (also herein termed RNAi agent, RNAi trigger, or trigger; e.g., double-stranded RNAi agents or small (or short) interfering RNA (siRNAs)), to hepatic cells in vivo. The delivery of such therapeutic oligonucleotides facilitates the selective and efficient inhibition of the expression of genes present in the liver, and specifically genes present in hepatocytes.

While metabolically stabilized NAG ligands have been previously suggested, reported data have shown no identifiable improvement or advantage over traditional standard NAG ligands. As supported by the Examples set forth herein, this is due to, at least in part, the failure of others to simultaneously stabilize the linkage to the oligonucleotide molecule through a linkage more stable than a traditional phosphodiester bond (e.g., the failure to introduce a more stable phosphorothioate or phosphorodithioate linkage), so that the benefit of the metabolically stabilized NAG ligand can be observed.

One aspect described herein is a compound or a pharmaceutically acceptable salt thereof, comprising:
a. an oligonucleotide between 12 and 49 nucleotides in length; and
b. a metabolically stabilized carbohydrate ligand;
wherein the double-stranded RNAi agent and the metabolically stabilized carbohydrate ligand are covalently bound by a phosphorothioate linkage, a phosphorodithioate linkage, or a linkage that is more metabolically stable than a phosphodiester linkage.

In another aspect described herein is a compound or a pharmaceutically acceptable salt thereof, comprising:
a. a double-stranded RNAi agent comprising a sense strand and an antisense strand, wherein the sense strand of the double-stranded RNAi agent comprises between 15 and 23 nucleotides, and the antisense strand of the double-stranded RNAi agent comprises between 18 and 23 nucleotides;
b. a metabolically stabilized carbohydrate ligand;
wherein the double-stranded RNAi agent and the metabolically stabilized carbohydrate ligand are covalently bound by a phosphorothioate linkage.

In some aspects, provided herein is a compound or a pharmaceutically acceptable salt thereof, comprising:
a. an oligonucleotide between 12 and 49 nucleotides in length; and
b. a metabolically stabilized carbohydrate ligand;
wherein the double-stranded RNAi agent and the metabolically stabilized carbohydrate ligand are covalently bound by a phosphorothioate linkage, a phosphorodithioate linkage, or a linkage that is more metabolically stable than a phosphodiester linkage;
wherein the metabolically stabilized carbohydrate ligand is of the formula:

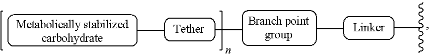

wherein:
each instance of the metabolically stabilized carbohydrate is independently a chemically modified carbohydrate moiety;
each instance of the tether is independently of the formula:

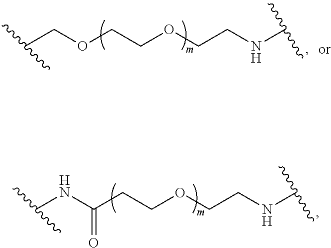

wherein m is an integer selected from 1 to 20 (e.g., 1, 2, 3, 4, 5, 6, 7, 8, 9, 10, 11, 12, 13, 14, 15, 16, 17, 18, 19 or 20);
the branch point group is a structure selected from the group consisting of:

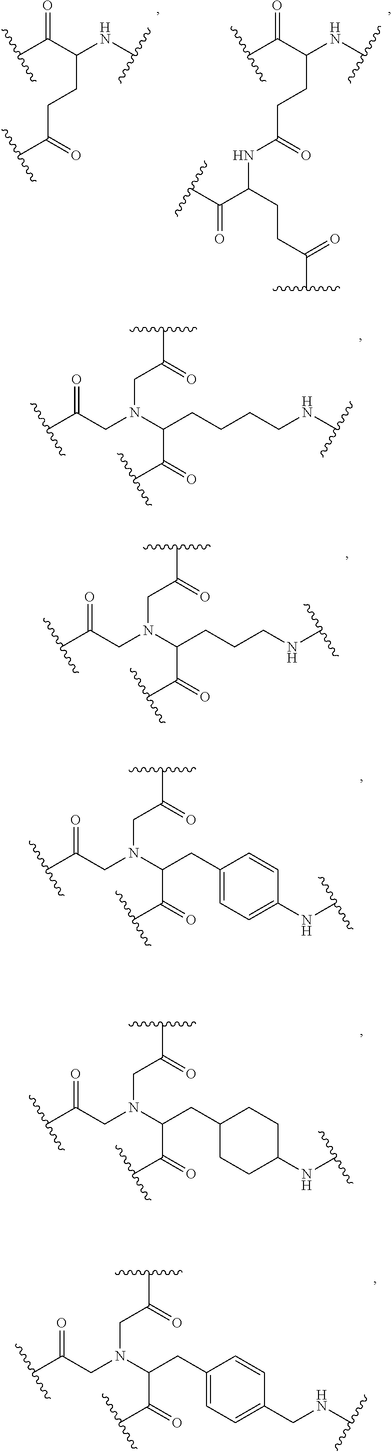

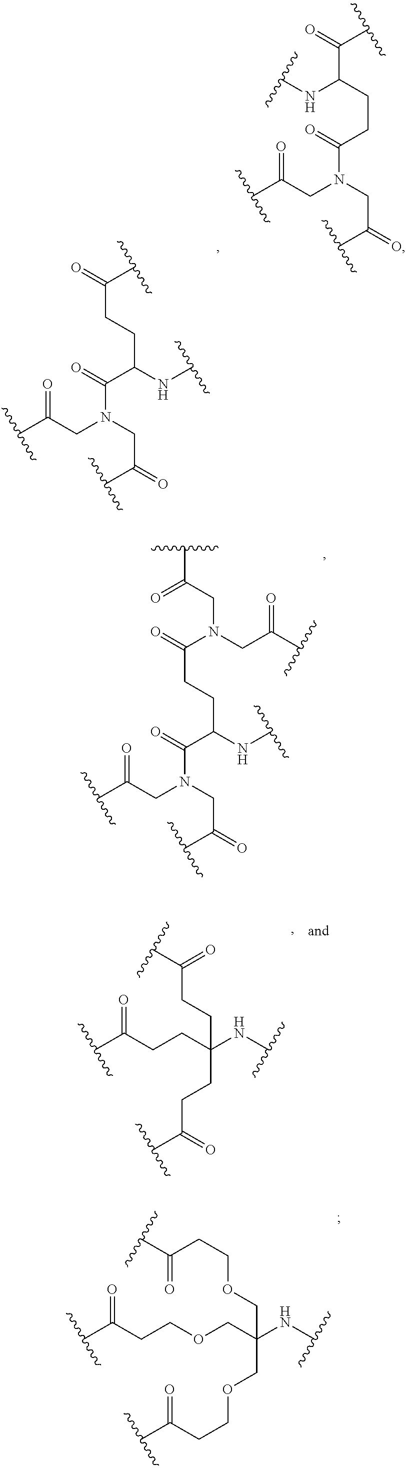 [SMILES: *NC(CCC(=O)N(CC(*)=O)CC(*)=O)C(*)=O ; *NC(CCC(*)=O)C(=O)N(CC(*)=O)CC(*)=O ; *NC(CCC(=O)N(CC(*)=O)CC(*)=O)C(=O)N(CC(*)=O)CC(*)=O ; *NC(CCC(*)=O)(CCC(*)=O)CCC(*)=O ; *NC(COCCC(*)=O)(COCCC(*)=O)COCCC(*)=O]

the linker is a structure selected from the group consisting of

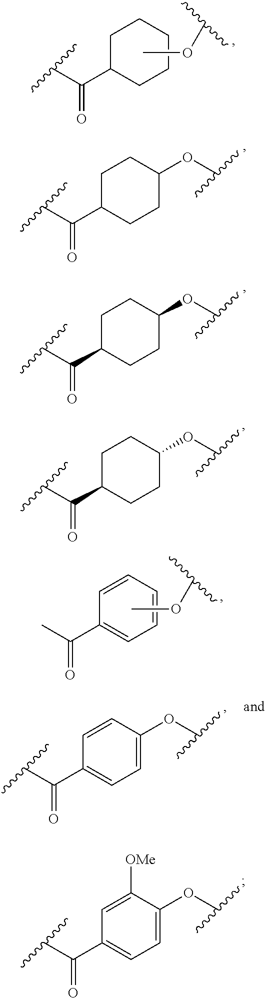

n is an integer from 1 to 4; and [symbol] indicates the attachment point to the oligonucleotide.

In some embodiments, the metabolically stabilized carbohydrate ligand comprises an N-Acetylgalactosamine (NAG or GalNAc) targeting ligand that includes the structure of the following Formulae:

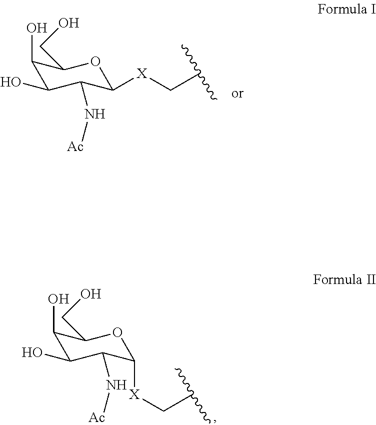

wherein X═CH₂ or S, and
an RNAi agent.
   As shown above, Formula I represents the β-anomeric linkage of a metabolically stabilized NAG ligand, while Formula II represents the α-anomeric linkage of a metabolically stabilized NAG ligand. The symbol

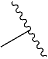

as used herein means that any group or groups may be linked thereto that is in accordance with the scope of the inventions described herein.

The metabolically stabilized carbohydrate ligand and the oligonucleotide may be covalently linked in any manner known in the art, provided that the linkers used to covalently link the various components are all metabolically stabilized linkages that are more stable in vivo than a phosphodiester bond. Exemplary embodiments of multimeric RNAi agent conjugates in accordance with the scope of the inventions disclosed herein may be found in the various examples herein.

Preferably, for the metabolically stabilized compound having the chemical structure of Formula I or Formula II, X is CH₂, as shown in the following Formula Ia and Formula IIa:

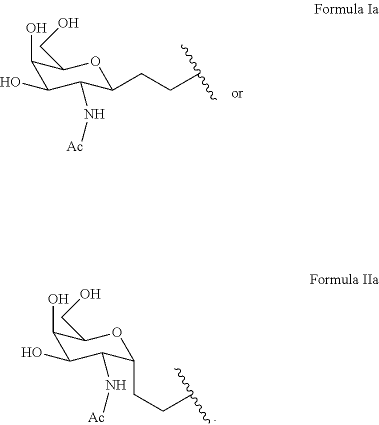 [SMILES: *CCC1OC(CO)C(O)C(O)C1NC(C)=O ; *CCC1OC(CO)C(O)C(O)C1NC(C)=O]

Preferably, the oligonucleotide is a double-stranded RNAi agent comprised of a sense strand and an antisense strand, and more preferably the metabolically stabilized carbohydrate ligand is linked to the sense strand of the RNAi agent.

While it has been previously reported that using metabolically stabilized NAG-RNAi agent conjugates can deliver an RNAi agent (specifically, an siRNA) to hepatocytes in vivo, previously reported results indicate that there is no difference with respect to asialoglycoprotein receptor (ASGPR) affinity or gene silencing activity compared to previously known, standard GalNAc ligands having the metabolically labile glycosidic linkage as shown in the following structure:

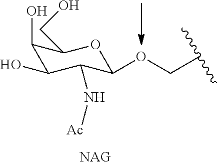 [SMILES: *COC1OC(CO)C(O)C(O)C1NC(C)=O]

(See Kandasmy et al., Metabolically stabilized Anomeric Linkages Containing GalNAc-siRNA Conjugates: An Interplay among ASGPR, Glycosidase, and RISC Pathways). As disclosed herein, this reported conclusion is inaccurate where, as supported by the data set forth in the Examples herein, the metabolically stabilized compounds having the chemical structure of Formula I or Formula II are covalently linked to the components by metabolically stabilized linkers that are more stable than a phosphodiester linkage (Compare, e.g., Id. at Abstract Figure of “Metabolically labile glycosidic linkage” (showing a labile phosphorothioate linkage to the RNAi agent and a monomeric-RNAi agent conjugate)).

In some embodiments, the metabolically stabilized compound having the structure of Formula I or Formula II is linked to the RNAi agent by a linker that is not a more labile linker than a phosphodiester linkages, such as a phosphorothioate linkage. In some embodiments, the metabolically stabilized compound having the structure of Formula I or Formula II is linked to the RNAi agents by a linker that includes a stable phosphorothioate linkage or a phosphorodithioate linkage.

In a further aspect of the disclosure herein, in some embodiments the length of the RNAi agents used in the multimeric RNAi agent conjugate delivery platform described herein are comprised of a duplex with a sense strand that is no more than 21 nucleotides in length, and an antisense strand that is no more than 21 nucleotides in length. In some embodiments, the multimeric RNAi agent conjugate delivery platform described herein are comprised of a duplex with a sense strand that is no more than 19 nucleotides in length, and an antisense strand that is no more than 19 nucleotides in length. As shown in the Examples set forth herein, the data show that additional delivery advantages can be attained when one or both the RNAi agents used in multimeric RNAi agent conjugate delivery platform is limited in length, preferably wherein the RNAi agent is comprised of sense strands and antisense strands that are no more than 21 nucleotides in length, no more than 20 nucleotides in length, or no more than 19 nucleotides in length.

The described metabolically stabilized carbohydrate conjugates can be used in methods for therapeutic treatment (including prophylactic, intervention, and preventative treatment) of conditions and diseases that can be mediated at least in part by the reduction in target gene expression, including, for example, diseases that can be mediated at least in part be the reduction of one or more genes expressed in hepatocytes. The compounds disclosed herein can selectively reduce target gene expression in cells in a subject, specifically in hepatocytes in the liver. The methods disclosed herein include, for example, the administration of one or more multimeric RNAi agent conjugates or one or more ASO conjugates to a subject, e.g., a human or animal subject, using any suitable methods known in the art, such as intravenous infusion, intravenous injection, or subcutaneous injection.

Also described herein are pharmaceutical compositions that include an oligonucleotide conjugate disclosed herein capable of inhibiting the expression of one or more target genes, wherein the composition further includes at least one pharmaceutically acceptable excipient.

The pharmaceutical compositions described herein that include one or more of the disclosed RNAi agents or other oligonucleotide conjugates or oligonucleotide-based agent conjugates are able to selectively and efficiently decrease or inhibit expression of a target gene in vivo.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### DETAILED DESCRIPTION

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a target mRNA.

As used herein, an “oligonucleotide-based agent” is a nucleotide sequence containing about 10-50 (e.g., 10 to 48, 10 to 46, 10 to 44, 10 to 42, 10 to 40, 10 to 38, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 50, 12 to 48, 12 to 46, 12 to 44, 12 to 42, 12 to 40, 12 to 38, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 50, 14 to 48, 14 to 46, 14 to 44, 14 to 42, 14 to 40, 14 to 38, 14 to 36, 14 to 34, 14 to 32, 14 to 30, 14 to 28, 14 to 26, 14 to 24, 14 to 22, 14 to 20, 14 to 18, 14 to 16, 16 to 50, 16 to 48, 16 to 46, 16 to 44, 16 to 42, 16 to 40, 16 to 38, 16 to 36, 16 to 34, 16 to 32, 16 to 30, 16 to 28, 16 to 26, 16 to 24, 16 to 22, 16 to 20, 16 to 18, 18 to 50, 18 to 48, 18 to 46, 18 to 44, 18 to 42, 18 to 40, 18 to 38, 18 to 36, 18 to 34, 18 to 32, 18 to 30, 18 to 28, 18 to 26, 18 to 24, 18 to 22, 18 to 20, 20 to 50, 20 to 48, 20 to 46, 20 to 44, 20 to 42, 20 to 40, 20 to 38, 20 to 36, 20 to 34, 20 to 32, 20 to 30, 20 to 28, 20 to 26, 20 to 24, 20 to 22, 22 to 50, 22 to 48, 22 to 46, 22 to 44, 22 to 42, 22 to 40, 22 to 38, 22 to 36, 22 to 34, 22 to 32, 22 to 30, 22 to 28, 22 to 26, 22 to 24, 24 to 50, 24 to 48, 24 to 46, 24 to 44, 24 to 42, 24 to 40, 24 to 38, 24 to 36, 24 to 34, 24 to 32, 24 to 30, 24 to 28, 24 to 26, 26 to 50, 26 to 48, 26 to 46, 26 to 44, 26 to 42, 26 to 40, 26 to 38, 26 to 36, 26 to 34, 26 to 32, 26 to 30, 26 to 28, 28 to 50, 28 to 48, 28 to 46, 28 to 44, 28 to 42, 28 to 40, 28 to 38, 28 to 36, 28 to 34, 28 to 32, to 28 to 30, 30 to 50, 30 to 48, 30 to 46, 30 to 44, 30 to 42, 30 to 40, 30 to 38, 30 to 36, 30 to 34, 30 to 32, 32 to 50, 32 to 48, 32 to 46, 32 to 44, 32 to 42, 32 to 40, 32 to 38, 32 to 36, 32 to 34, 34 to 50, 34 to 48, 34 to 46, 34 to 44, 34 to 42, 34 to 40, 34 to 38, 34 to 36, 36 to 50, 36 to 48, 36 to 46, 36 to 44, 36 to 42, 36 to 40, 36 to 38, 38 to 50, 38 to 48, 38 to 46, 38 to 44, 38 to 42, 38 to 40, 40 to 50, 40 to 48, 40 to 46, 40 to 44, 40 to 42, 42 to 50, 42 to 48, 42 to 46, 42 to 44, 44 to 50, 44 to 48, 44 to 46, 46 to 50, 46 to 48, or 48 to 50) nucleotides or nucleotide base pairs. In some embodiments, an oligonucleotide-based agent has a nucleobase sequence that is at least partially complementary to a coding sequence in an expressed target nucleic acid or target gene within a cell. In some embodiments, the oligonucleotide-based agent, upon delivery to a cell expressing a gene, are able to inhibit the expression of the underlying gene, and are referred to herein as “expression-inhibiting oligonucleotide-based agents.” The gene expression can be inhibited in vitro or in vivo.

“Oligonucleotide-based agents” include, but are not limited to: single-stranded oligonucleotides, single-stranded antisense oligonucleotides (ASOs), short interfering RNAs (siRNAs), double-strand RNAs (dsRNA), micro RNAs (miRNAs), short hairpin RNAs (shRNA), ribozymes, interfering RNA molecules, and dicer substrates. In some embodiments, an oligonucleotide-based agent is a single-stranded oligonucleotide, such as an antisense oligonucleotide. In some embodiments, an oligonucleotide-based agent is a double-stranded oligonucleotide. In some embodiments, an oligonucleotide-based agent is a double-stranded oligonucleotide that is an RNAi agent.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the preventative treatment, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

As used herein, the term “linked” or “conjugated” when referring to the connection between two compounds or molecules means that two molecules are joined by a covalent bond or are associated via noncovalent bonds (e.g., hydrogen bonds or ionic bonds). In some examples, where the term “linked” or “conjugated” refers to the association between two molecules via noncovalent bonds, the association between the two different molecules has a K_{D} of less than 1×10⁻⁴ M (e.g., less than 1×10⁻⁵ M, less than 1×10⁻⁶ M, or less than 1×10⁻⁷ M) in physiologically acceptable buffer (e.g., buffered saline). Unless stated, the terms “linked” and “conjugated” as used herein may refer to the connection between a first compound and a second compound either with or without any intervening atoms or groups of atoms.

As used herein, a linking group is one or more atoms that connects one molecule or portion of a molecule to another to second molecule or second portion of a molecule. Similarly, as used in the art, the term scaffold is sometimes used interchangeably with a linking group. Linking groups may comprise any number of atoms or functional groups. In some embodiments, linking groups may not facilitate any biological or pharmaceutical response, and merely serve to link two biologically active molecules.

As used herein, a “metabolically stabilized carbohydrate ligand” is a carbohydrate ligand suitable for binding to the asialoglycoprotein receptor that is abundantly expressed on hepatocytes, wherein the carbohydrate ligand has been chemically modified to provide for a more stable chemical composition in serum (e.g., human serum). Suitable tests to determine whether such a compound is more metabolically stabilized (e.g., more stable in human serum) compared a to carbohydrate ligand that has not been chemically modified and can still retain the ability to deliver cargo molecules such as RNAi agents to hepatocytes can be readily determined by persons of skill in the art.

Certain chemical modifications that provide metabolically stabilized carbohydrate ligand, include but are not limited to, modifications at the atom adjacent to the anomeric carbon, or modifications to the phosphodiester linkage connecting the metabolically stabilized carbohydrate ligand to an oligonucleotide (e.g., a phosphorothioate linkage or phosphorodithioate linkage). In some embodiments, the metabolically stabilized carbohydrate ligand is chemically modified at the atom adjacent to the anomeric carbon. In some embodiments, the atom adjacent to the anomeric carbon is a second carbon atom, which may be a methylene (—CH2-) moiety. In other embodiments, the atom adjacent to the anomeric carbon is a sulfur (—S—) atom.

In some embodiments, metabolically stable carbohydrate ligands comprise a sugar moiety. In some embodiments, the sugar moiety is selected from the group consisting of glucose, galactose, and N-Acetylgalactosamine. Non-limiting examples of a metabolically stabilized carbohydrate ligands are the metabolically stabilized N-Acetylgalactosamine ligands of Formula I and Formula II disclosed herein.

In some embodiments, a metabolically stabilized carbohydrate ligand is 1.5× more stable in human serum than a non-metabolically stabilized carbohydrate ligand as measured by compound half-life in serum in vitro. In some embodiments, a metabolically stabilized carbohydrate ligand is 2× more stable in human serum than a non-metabolically stabilized carbohydrate ligand as measured by compound half-life in serum in vitro. In some embodiments, a metabolically stabilized carbohydrate ligand is 5× more stable in human serum than a non-metabolically stabilized carbohydrate ligand as measured by compound half-life in serum in vitro. In some embodiments, a metabolically stabilized carbohydrate ligand is 10× more stable in human serum than a non-metabolically stabilized carbohydrate ligand as measured by compound half-life in serum in vitro.

Unless stated otherwise, the symbol

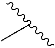

as used herein means that any group or groups may be linked thereto that is in accordance with the scope of the inventions described herein.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The term “pharmaceutically acceptable salt” refers to those salts which are, within the scope of sound medical judgment, suitable for use in contact with the tissues of humans without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference.

Pharmaceutically acceptable salts of the compounds of this disclosure include those derived from suitable inorganic and organic acids and bases. Examples of pharmaceutically acceptable, nontoxic acid addition salts are salts formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and alkyl ammonium (i.e., (C₁₋₄ alkyl)₄N⁺) salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### Modified Nucleotides

In some embodiments, an RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as N_{UNA} or NUNA), locked nucleotides (represented herein as N_{LNA} or NLNA), 3′-O-methoxy (2′ internucleoside linked) nucleotides (represented herein as 3′-OMen), 2′-F-Arabino nucleotides (represented herein as NfANA or Nf_{ANA}), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides, and represented herein as a lower case letter ‘n’ in a nucleotide sequence), 2′-fluoro nucleotides (also referred to herein as 2′-deoxy-2′-fluoro nucleotide, and represented herein as Nf), 2′-deoxy nucleotides (represented herein as dN), 2′-methoxyethyl (2′-O-2-methoxylethyl) nucleotides (also referred to herein as 2′-MOE, and represented herein as NM), 2′-amino nucleotides, and 2′-alkyl nucleotides. It is not necessary for all positions in a given compound to be uniformly modified. Conversely, more than one modification can be incorporated in a single RNAi agent or even in a single nucleotide thereof. The RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some embodiments, one or more nucleotides of an RNAi agent is an unmodified ribonucleotide.

### Modified Internucleoside Linkages

In some embodiments, one or more nucleotides of an RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an RNAi agent can contain 1, 2, 3, 4, 5, or 6 phosphorothioate linkages, an antisense strand of an RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an RNAi agent can contain 1, 2, 3, or 4 phosphorothioate linkages, an antisense strand of an RNAi agent can contain 1, 2, 3, or 4 phosphorothioate linkages, or both the sense strand and the antisense strand independently can contain 1, 2, 3, or 4 phosphorothioate linkages.

In some embodiments, an RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, one phosphorothioate internucleoside linkage is at the 5′ end of the sense strand, and another phosphorothioate linkage is at the 3′ end of the sense strand. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, an RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 17-19, 18-20, 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, an RNAi agent contains at least three or four phosphorothioate internucleoside linkages in the antisense strand.

In some embodiments, an RNAi agent contains one or more modified nucleotides and one or more modified internucleoside linkages. In some embodiments, a 2′-modified nucleoside is combined with modified internucleoside linkage.

### Targeting Ligands and Targeting Groups

As disclosed herein, the multimeric RNAi agent conjugate delivery platform is comprised of one or more targeting groups. Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules.

In some embodiments, a targeting group is linked to an RNAi agent using a linker, such as a PEG linker or one, two, or three abasic and/or ribitol (abasic ribose) residues, which in some instances can serve as linkers. In some embodiments, a targeting group is covalently linked to the 3′ and/or 5′ end of either the sense strand and/or the antisense strand of an RNAi agent. In some embodiments, a targeting ligand is linked to the 3′ and/or 5′ end of the sense strand of one of the RNAi agents. In some embodiments, a targeting group is linked to the 5′ end of an RNAi agent sense strand of one RNAi agent. In some embodiments, a targeting group is linked internally to one or more nucleotides of an RNAi agent sense strand. In some embodiments, a targeting ligand is positioned between two RNAi agents in the multimeric RNAi agent conjugate. A targeting group may be linked directly or indirectly to the RNAi agent via a linker/linking group. In some embodiments, a targeting group is linked to the RNAi agent via a metabolically stabilized bond or linkage.

In some embodiments, a targeting group comprises an asialoglycoprotein receptor ligand. As used herein, an asialoglycoprotein receptor ligand is a ligand that contains a moiety having affinity for the asialoglycoprotein receptor. As noted herein, the asialoglycoprotein receptor is highly expressed on hepatocytes. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formylgalactosamine, N-Acetylgalactosamine, N-propionyl-galactosamine, N-n-butanoyl-galactosamine, and N-iso-butanoylgalactos-amine (see for example: S. T. Iobst and K. Drickamer, J. B. C., 1996, 271, 6686), as well as metabolically stabilized glycosidic linked N-Acetylgalactosamine. Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945).

Galactose derivatives have been used to target molecules to hepatocytes in vivo through their binding to the asialoglycoprotein receptor expressed on the surface of hepatocytes. Binding of asialoglycoprotein receptor ligands to the asialoglycoprotein receptor(s) facilitates cell-specific targeting to hepatocytes and endocytosis of the molecule into hepatocytes. Asialoglycoprotein receptor ligands can be monomeric (e.g., having a single galactose derivative, also referred to as monovalent or monodentate) or multimeric (e.g., having multiple galactose derivatives). The galactose derivative or galactose derivative cluster can be attached to the 3′ or 5′ end of the sense or antisense strand of the RNAi agent using methods known in the art. The galactose derivative or galactose derivative cluster can also be attached internally to one or more nucleotides the sense or antisense strand of the RNAi agent using methods known in the art.

In some embodiments, the targeting ligand is comprised of one or more metabolically stabilized N-Acetylgalactosamine (NAG or GalNAc) targeting ligands that includes the structure of the following Formulae:

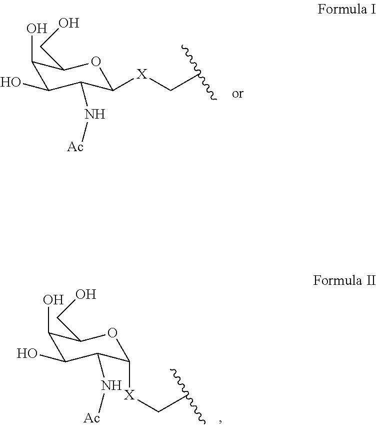

wherein X═CH₂ or S.

In some embodiments, the metabolically stabilized NAG targeting ligand is a trimer (also referred to as tri-antennary or tri-valent), wherein three moieties of Formula I or Formula II are attached through a centralized branch point. (See, e.g., the chemical structure referred to herein of NAG52). In some embodiments, the targeting ligand is a cluster of four metabolically stabilized NAG moieties thereby forming a tetramer (also referred to as tetra-antennary or tetra-valent) targeting ligand. In some embodiments, the metabolically stabilized NAG targeting ligand is a bi-antennary or bi-valent), wherein two moieties of Formula I or Formula II are attached through a centralized branch point.

As used herein, a metabolically stabilized NAG targeting ligand contains one or more moieties of Formula I or Formula II, each linked to a central branch point. In some embodiments, the targeting ligands are linked to the branch point via linkers or spacers. In some embodiments, the linker or spacer is a flexible hydrophilic spacer, such as a PEG group (see, e.g., U.S. Pat. No. 5,885,968; Biessen et al. J. Med. Chem. 1995 Vol. 39 p. 1538-1546). The branch point can be any small molecule which permits attachment of three galactose derivatives and further permits attachment of the branch point to an RNAi agent.

An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group.

In some embodiments, a delivery platform or compound disclosed herein comprises one or more targeting ligands that include a compound of Formula I or Formula II:

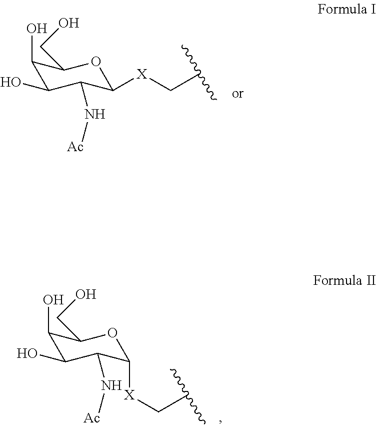

or a pharmaceutically acceptable salt thereof,
wherein X═CH₂ or S.

In some embodiments, a delivery platform or compound disclosed herein comprises one or more targeting ligands that include a compound of Formula Ia or Formula Ib:

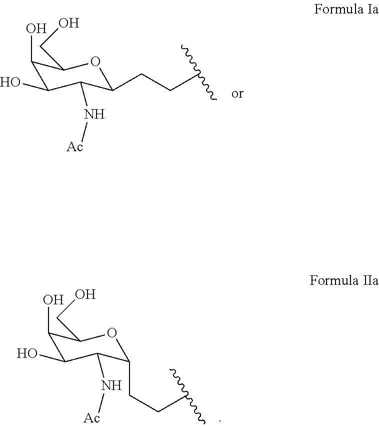 [SMILES: *CCC1OC(CO)C(O)C(O)C1NC(C)=O ; *CCC1OC(CO)C(O)C(O)C1NC(C)=O]

Methods of making compounds of Formula Ia are described in the Examples below.

In some embodiments, compounds that may be conjugated to RNAi agents to synthesize a delivery platform for an RNAi agent are shown in Table 1 below.

In some embodiments, a compound described herein comprises:
a. an oligonucleotide between 12 and 49 nucleotides in length; and
b. a metabolically stabilized carbohydrate ligand;
wherein the double-stranded RNAi agent and the metabolically stabilized carbohydrate ligand are covalently bound by a phosphorothioate linkage, a phosphorodithioate linkage, or a linkage that is more metabolically stable than a phosphodiester linkage;
wherein the metabolically stabilized carbohydrate ligand is of the formula:

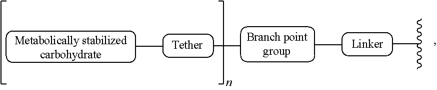

wherein:
each instance of the metabolically stabilized carbohydrate is independently a chemically modified carbohydrate moiety; each instance of the tether is independently of the formula:

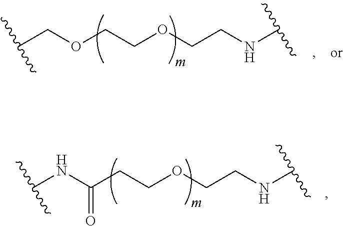

wherein m is an integer selected from 1 to 20 (e.g., 1, 2, 3, 4, 5, 6, 7, 8, 9, 10, 11, 12, 13, 14, 15, 16, 17, 18, 19 or 20);
the branch point group is a structure selected from the group consisting of:

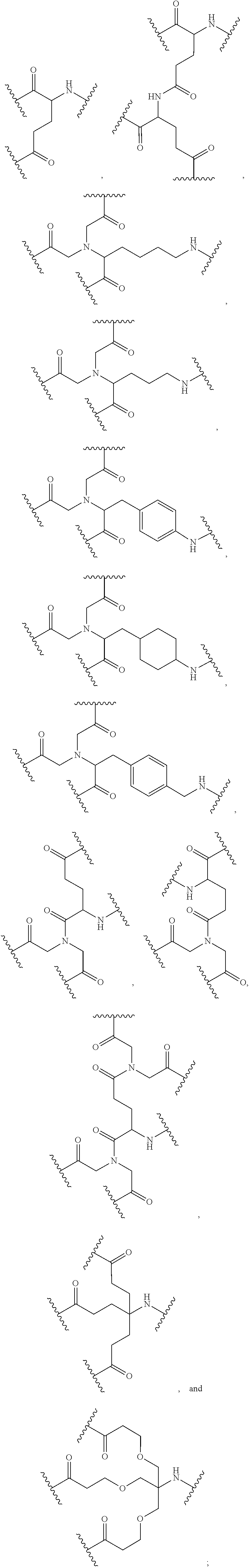

the linker is a structure selected from the group consisting of:

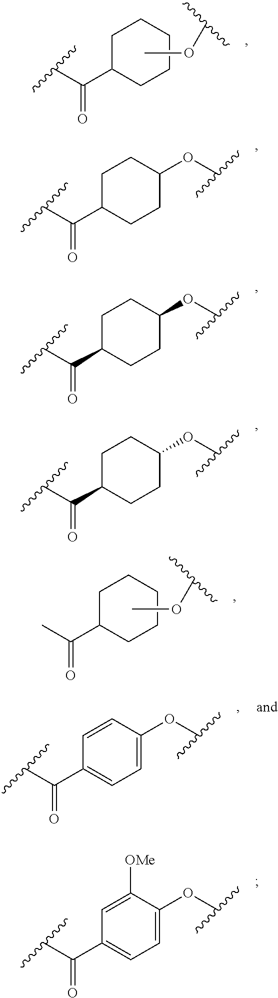

n is an integer from 1 to 4; and [symbol] indicates the attachment point to the oligonucleotide.

In certain embodiments, the tether is of the formula:

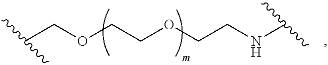

wherein m is an integer selected from 1 to 20 (e.g., 1, 2, 3, 4, 5, 6, 7, 8, 9, 10, 11, 12, 13, 14, 15, 16, 17, 18, 19 or 20). In certain embodiments, the tether is of the formula:

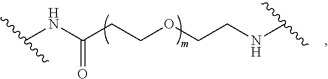

wherein m is an integer selected from 1 to 20 (e.g., 1, 2, 3, 4, 5, 6, 7, 8, 9, 10, 11, 12, 13, 14, 15, 16, 17, 18, 19 or 20). In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 13. In certain embodiments, m is 14. In certain embodiments, m is 15. In certain embodiments, m is 16. In certain embodiments, m is 17. In certain embodiments, m is 18. In certain embodiments, m is 19. In certain embodiments, m is 20.

In certain embodiments, the tether is of the formula:

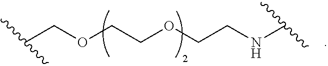

In certain embodiments, the tether is of the formula:

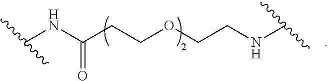

In certain embodiments, the branch point group is of the formula:

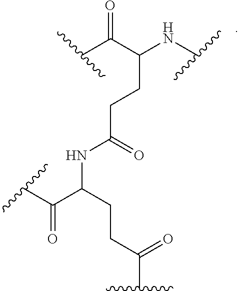 [SMILES: *NC(CCC(=O)NC(CCC(*)=O)C(*)=O)C(*)=O]

In certain embodiments, the branch point group is of the formula:

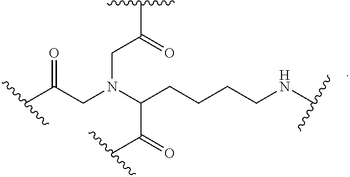 [SMILES: *NCCCCC(C(*)=O)N(CC(*)=O)CC(*)=O]

In certain embodiments, the branch point group is of the formula:

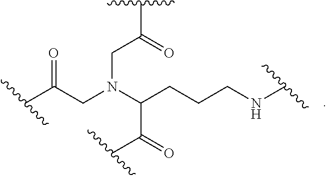 [SMILES: *NCCCC(C(*)=O)N(CC(*)=O)CC(*)=O]

In certain embodiments, the branch point group is of the formula:

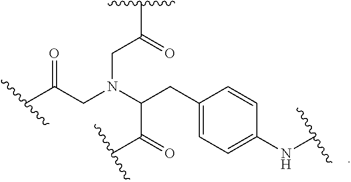 [SMILES: *Nc1ccc(CC(C(*)=O)N(CC(*)=O)CC(*)=O)cc1]

In certain embodiments, the branch point group is of the formula:

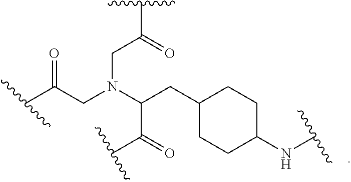 [SMILES: *NC1CCC(CC(C(*)=O)N(CC(*)=O)CC(*)=O)CC1]

In certain embodiments, the branch point group is of the formula:

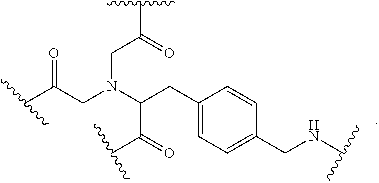 [SMILES: *NCc1ccc(CC(C(*)=O)N(CC(*)=O)CC(*)=O)cc1]

In certain embodiments, the branch point group is of the formula:

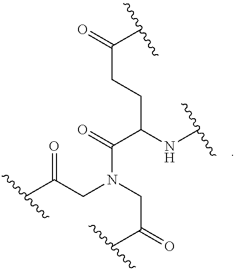 [SMILES: *NC(CCC(*)=O)C(=O)N(CC(*)=O)CC(*)=O]

In certain embodiments, the branch point group is of the formula:

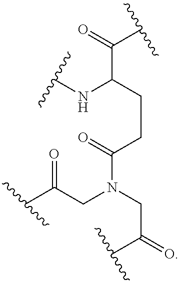 [SMILES: *NC(CCC(=O)N(CC(*)=O)CC(*)=O)C(*)=O]

In certain embodiments, the branch point group is of the formula:

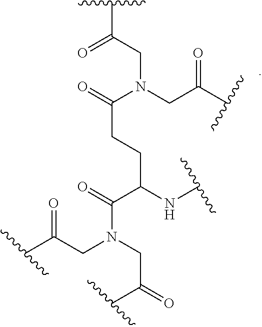 [SMILES: *NC(CCC(=O)N(CC(*)=O)CC(*)=O)C(=O)N(CC(*)=O)CC(*)=O]

In certain embodiments, the branch point group is of the formula:

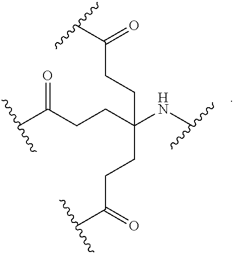 [SMILES: *NC(CCC(*)=O)(CCC(*)=O)CCC(*)=O]

In certain embodiments, the branch point group is of the formula:

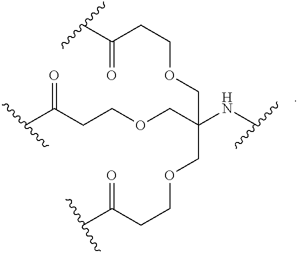 [SMILES: *NC(COCCC(*)=O)(COCCC(*)=O)COCCC(*)=O]

In certain embodiments, the branch point group is of the formula:

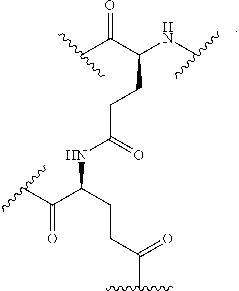 [SMILES: *N[C@@H](CCC(=O)N[C@@H](CCC(*)=O)C(*)=O)C(*)=O]

In certain embodiments, the branch point group is of the formula:

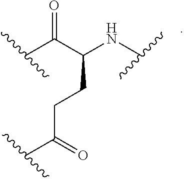 [SMILES: *N[C@@H](CCC(*)=O)C(*)=O]

In certain embodiments, the linker structure is of the formula:

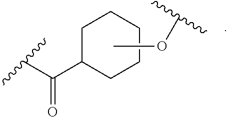

In certain embodiments, the linker structure is of the formula:

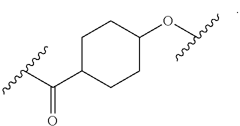 [SMILES: *OC1CCC(C(*)=O)CC1]

In certain embodiments, the linker structure is of the formula:

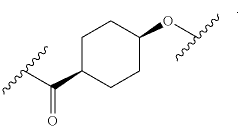 [SMILES: *O[C@H]1CC[C@@H](C(*)=O)CC1]

In certain embodiments, the linker structure is of the formula:

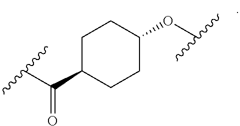 [SMILES: *O[C@H]1CC[C@H](C(*)=O)CC1]

In certain embodiments, the linker structure is of the formula:

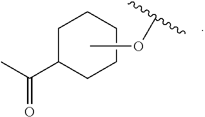

In certain embodiments, the linker structure is of the formula:

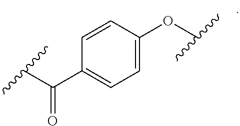 [SMILES: *Oc1ccc(C(*)=O)cc1]

In certain embodiments, the linker structure is of the formula:

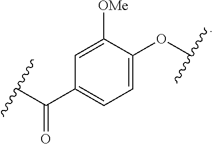 [SMILES: *Oc1ccc(C(*)=O)cc1OC]

In certain embodiments, n is 1. In certain embodiments, n is 2. In certain embodiments, n is 3. In certain embodiments, n is 4.

In some embodiments, a compound described herein comprises any of the metabolically stabilized N-Acetylgalactosamine targeting ligands shown in Table 1 below.

TABLE 1
Examples of metabolically stabilized N-Acetylgalactosamine targeting ligands.

| | |
|---|---|
| NAG42s | 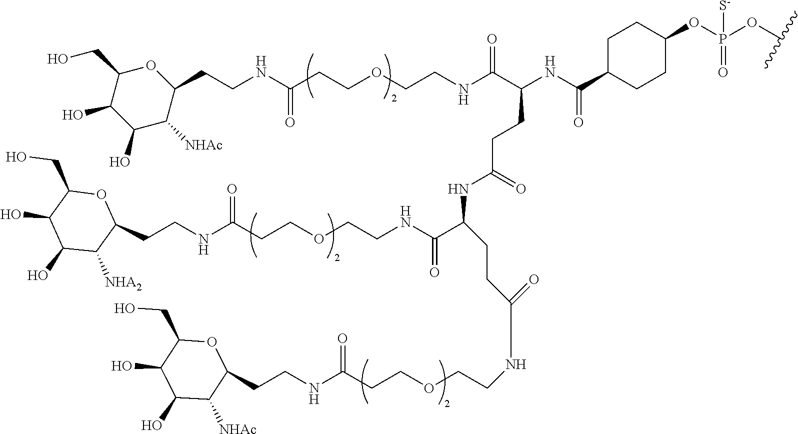 |
| NAG52s | 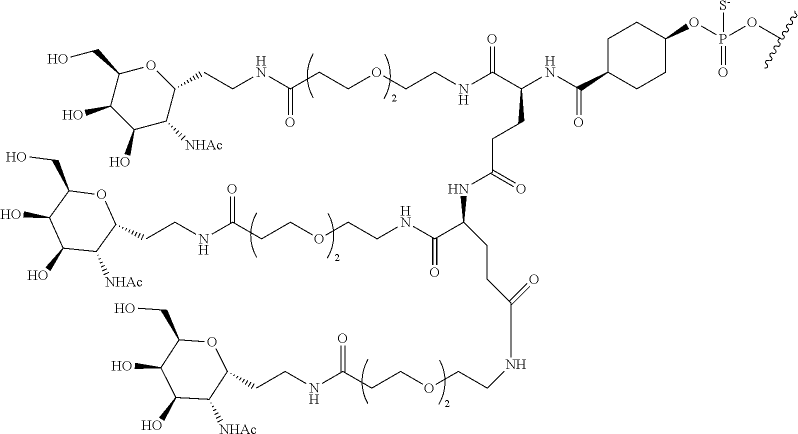 |
| NAG55s | 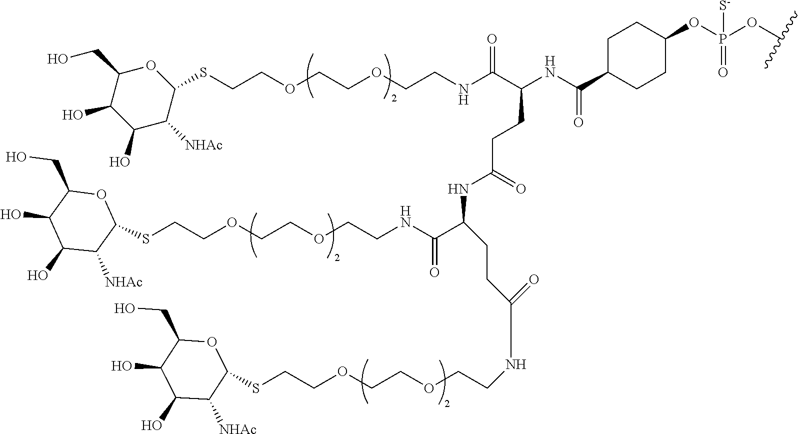 |
| NAG1008s | 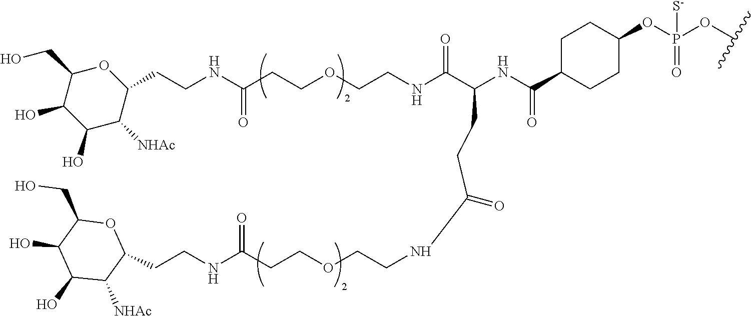 |

### Linking Groups

In some embodiments, an RNAi agent contains or is conjugated to one or more non-nucleotide groups including, but not limited to a linking group a delivery polymer, or a delivery vehicle. The non-nucleotide group can enhance targeting, delivery, or attachment of the RNAi agent. Examples of linking groups are provided in Table 2. The non-nucleotide group can be covalently linked to the 3′ and/or 5′ end of either the sense strand and/or the antisense strand.

In some embodiments, an RNAi agent contains a non-nucleotide group linked to the 3′ and/or 5′ end of the sense strand. In some embodiments, a non-nucleotide group is linked to the 5′ end of an RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

The RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

A linker or linking group is a connection between two atoms that links one chemical group (such as an RNAi agent) or segment of interest to another chemical group (such as a targeting ligand, targeting group, PK/PD modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, targeting groups are linked to RNAi agents without the use of an additional linker. In some embodiments, the targeting group is designed having a linker readily present to facilitate the linkage to an RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group may be conjugated synthetically to the 5′ or 3′ end of the sense strand of an RNAi agent described herein. In some embodiments, a linking group is conjugated synthetically to the 5′ end of the sense strand of an RNAi agent. In some embodiments, a linking group conjugated to an RNAi agent may be a trialkyne linking group.

Examples of certain modified nucleotides and linking groups, are provided in Table 2.

TABLE 2
Structures Representing Various Modified Nucleotides and Linking Groups

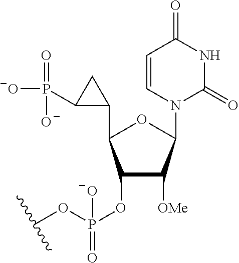

cPrpu

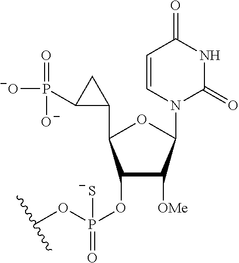

cPrpus

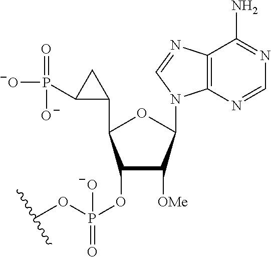

cPrpa

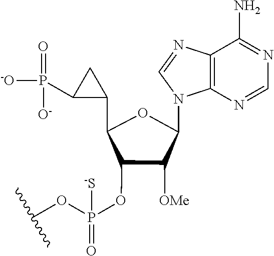

cPrpas

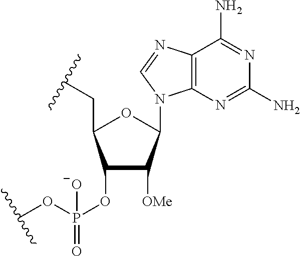

a_2N

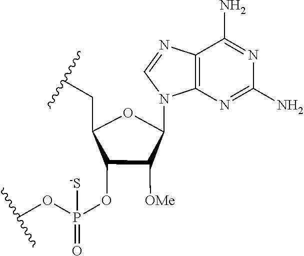

a_2Ns
When positioned internally:
linkage towards 5′ end

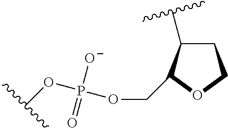

linkage towards 3′ end
(invAb)
linkage towards 5′ end

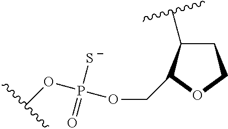

linkage towards 3′ end
(invAb)s
When positioned at the 3' terminal end:
linkage towards 5' end

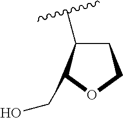

(invAb)
When position at the 3′ terminal end:
linkage towards 5′ end

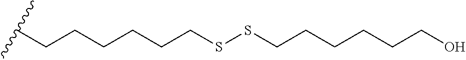 [SMILES: *CCCCCCSSCCCCCCO]

(C6-SS-C6)
When positioned internally:
linkage towards 5′ end linkage towards 3′ end

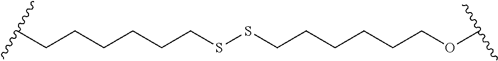 [SMILES: *CCCCCCSSCCCCCCO*]

(C6-SS-C6)
When position at the 3′ terminal end:
linkage towards 5′ end

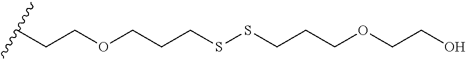 [SMILES: *CCOCCCSSCCCOCCO]

(6-SS-6)
When positioned internally:
linkage towards 5′ end linkage towards 3′ end

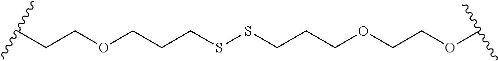 [SMILES: *CCOCCCSSCCCOCCO*]

(6-SS-6)

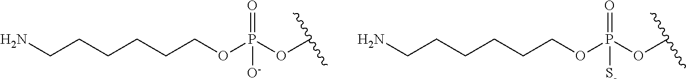

(NH2-C6) (NH2-C6)s

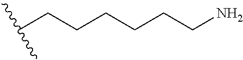 [SMILES: *CCCCCCN]

(C6-NH2)

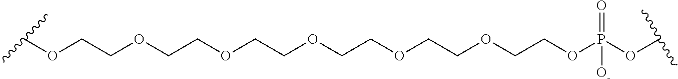

Sp18

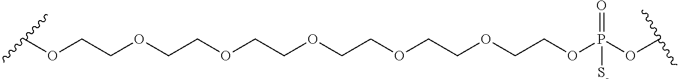

Sp18s

Alternatively, other linking groups known in the art may be used.

In addition or alternatively to linking an RNAi agent to one or more targeting ligands, targeting groups, and/or PK/PD modulators, in some embodiments, a delivery vehicle may be used to deliver an RNAi agent to a cell or tissue. A delivery vehicle is a compound that can improve delivery of the RNAi agent to a cell or tissue, and can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/022309, WO 2011/104169, and WO 2012/083185, WO 2013/032829, WO 2013/158141, each of which is incorporated herein by reference), or other delivery systems available in the art.

### Pharmaceutical Compositions

In some embodiments, the present disclosure provides pharmaceutical compositions that include, consist of, or consist essentially of, one or more of the delivery platforms disclosed herein.

As used herein, a “pharmaceutical composition” comprises a pharmacologically effective amount of an Active Pharmaceutical Ingredient (API), and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions described herein can contain other additional components commonly found in pharmaceutical compositions. In some embodiments, the additional component is a pharmaceutically-active material. Pharmaceutically-active materials include, but are not limited to: anti-pruritics, astringents, local anesthetics, or anti-inflammatory agents (e.g., antihistamine, diphenhydramine, etc.), small molecule drug, antibody, antibody fragment, aptamers, and/or vaccines.

The pharmaceutical compositions may also contain preserving agents, solubilizing agents, stabilizing agents, wetting agents, emulsifiers, sweeteners, colorants, odorants, salts for the variation of osmotic pressure, buffers, coating agents, or antioxidants. They may also contain other agent with a known therapeutic benefit.

The pharmaceutical compositions can be administered in a number of ways depending upon whether local or systemic treatment is desired and upon the area to be treated. Administration can be made by any way commonly known in the art, such as, but not limited to, topical (e.g., by a transdermal patch), pulmonary (e.g., by inhalation or insufflation of powders or aerosols, including by nebulizer, intratracheal, intranasal), epidermal, transdermal, oral or parenteral. Parenteral administration includes, but is not limited to, intravenous, intraarterial, subcutaneous, intraperitoneal or intramuscular injection or infusion; subdermal (e.g., via an implanted device), intracranial, intraparenchymal, intrathecal, and intraventricular, administration. In some embodiments, the pharmaceutical compositions described herein are administered by subcutaneous injection. The pharmaceutical compositions may be administered orally, for example in the form of tablets, coated tablets, dragées, hard or soft gelatin capsules, solutions, emulsions or suspensions. Administration can also be carried out rectally, for example using suppositories; locally or percutaneously, for example using ointments, creams, gels, or solutions; or parenterally, for example using injectable solutions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of any of the ligands described herein that can be in microcrystalline form, for example, in the form of an aqueous microcrystalline suspension. Liposomal formulations or biodegradable polymer systems can also be used to present any of the ligands described herein for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). As used herein, “pharmacologically effective amount,” “therapeutically effective amount,” or simply “effective amount” refers to that amount of an the pharmaceutically active agent to produce a pharmacological, therapeutic or preventive result.

Medicaments containing a RNAi agent are also an object of the present invention, as are processes for the manufacture of such medicaments, which processes comprise bringing one or more compounds containing a RNAi agent, and, if desired, one or more other substances with a known therapeutic benefit, into a pharmaceutically acceptable form.

The described RNAi agents and pharmaceutical compositions comprising RNAi agents disclosed herein may be packaged or included in a kit, container, pack, or dispenser. The RNAi agents and pharmaceutical compositions comprising the RNAi agents may be packaged in pre-filled syringes or vials.

### Methods of Treatment and Inhibition of Expression

The delivery platforms disclosed herein can be used to treat a subject (e.g., a human or other mammal) having a disease or disorder that would benefit from administration of the RNAi agent. In some embodiments, the delivery platforms for an RNAi agent disclosed herein can be used to treat a subject (e.g., a human) that would benefit from reduction and/or inhibition in expression of mRNA and/or a target protein levels.

In some embodiments, the subject is administered a therapeutically effective amount of any one or more RNAi agents. Treatment of a subject can include therapeutic and/or prophylactic treatment. The subject is administered a therapeutically effective amount of any one or more RNAi agents described herein. The subject can be a human, patient, or human patient. The subject may be an adult, adolescent, child, or infant. Administration of a pharmaceutical composition described herein can be to a human being or animal.

The RNAi agents described herein can be used to treat at least one symptom in a subject having a disease or disorder relating to a target gene, or having a disease or disorder that is mediated at least in part by target gene expression. In some embodiments, the RNAi agents are used to treat or manage a clinical presentation of a subject with a disease or disorder that would benefit from or be mediated at least in party by a reduction in target mRNA. The subject is administered a therapeutically effective amount of one or more of the RNAi agents or RNAi agent-containing compositions described herein. In some embodiments, the methods disclosed herein comprise administering a composition comprising an RNAi agent described herein to a subject to be treated. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by target gene expression, in a patient in need thereof, wherein the methods include administering to the patient any of the RNAi agents described herein.

In some embodiments, the gene expression level and/or mRNA level of a target gene in a subject to whom an RNAi agent is administered is reduced by at least about 30%, 35%, 40%, 45%, 50%, 55%, 60%, 65%, 70%, 75%, 80%, 85%, 95%, 96%, 97%, 98%, 99%, or greater than 99% relative to the subject prior to being administered the RNAi agent or to a subject not receiving the RNAi agent. The gene expression level and/or mRNA level in the subject may be reduced in a cell, group of cells, and/or tissue of the subject.

In some embodiments, the protein level in a subject to whom an RNAi agent has been administered is reduced by at least about 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the RNAi agent or to a subject not receiving the RNAi agent. The protein level in the subject may be reduced in a cell, group of cells, tissue, blood, and/or other fluid of the subject.

A reduction in mRNA levels and protein levels can be assessed by any methods known in the art. As used herein, a reduction or decrease in mRNA level and/or protein level are collectively referred to herein as a reduction or decrease in the target gene or inhibiting or reducing the expression of the target gene. The Examples set forth herein illustrate known methods for assessing inhibition of gene expression.

In some embodiments, RNAi agents may be used in the preparation of a pharmaceutical composition for use in the treatment of a disease, disorder, or symptom that is mediated at least in part by target gene expression.

In some embodiments, methods of treating a subject are dependent on the body weight of the subject. In some embodiments, RNAi agents may be administered at a dose of about 0.05 mg/kg to about 40.0 mg/kg of body weight of the subject. In other embodiments RNAi agents may be administered at a dose of about 5 mg/kg to about 20 mg/kg of body weight of the subject.

In some embodiments, RNAi agents may be administered in a split dose, meaning that two doses are given to a subject in a short (for example, less than 24 hour) time period. In some embodiments, about half of the desired daily amount is administered in an initial administration, and the remaining about half of the desired daily amount is administered approximately four hours after the initial administration.

In some embodiments, RNAi agents may be administered once a week (i.e., weekly). In other embodiments, RNAi agents may be administered biweekly (once every other week).

In some embodiments, RNAi agents or compositions containing RNAi agents may be used for the treatment of a disease, disorder, or symptom that is mediated at least in part by target gene expression.

### Cells, Tissues, and Non-Human Organisms

Cells, tissues, and non-human organisms that include at least one of the delivery platforms comprising an RNAi agent described herein is contemplated. The cell, tissue, or non-human organism is made by delivering the RNAi agent to the cell, tissue, or non-human organism by any means available in the art. In some embodiments, the cell is a mammalian cell, including, but not limited to, a human cell.

The above provided embodiments and items are now illustrated with the following, non-limiting examples.

### Illustrative Embodiments

Provided here are illustrative embodiments of the disclosed technology. These embodiments are illustrative only and do not limit the scope of the present disclosure or of the claims attached hereto.

Embodiment 1. A compound for inhibiting expression of one or more genes, comprising:
a. an oligonucleotide comprising a strand 12-49 nucleotides in length; and
b. a metabolically stabilized carbohydrate ligand;
wherein the oligonucleotide and the metabolically stabilized carbohydrate ligand are covalently bound by a phosphorothioate linkage, a phosphorodithioate linkage, or another linkage that is more metabolically stable than a phosphodiester linkage, or a pharmaceutically acceptable salt thereof.

Embodiment 2. The compound of Embodiment 1 or a pharmaceutically acceptable salt thereof, wherein the metabolically stabilized carbohydrate ligand is of the formula:

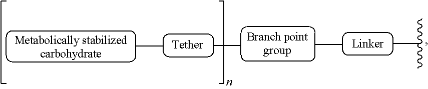

wherein:
each instance of the metabolically stabilized carbohydrate is independently a chemically modified carbohydrate moiety;
each instance of the tether is independently of the formula:

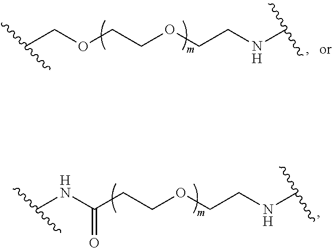

wherein m is an integer selected from 1 to 20;
the branch point group is a structure selected from the group consisting of:

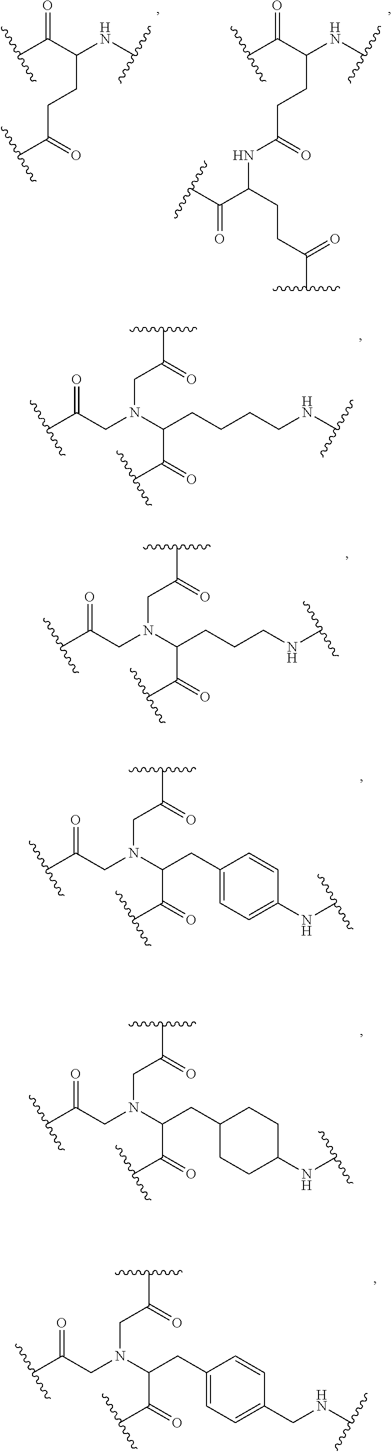

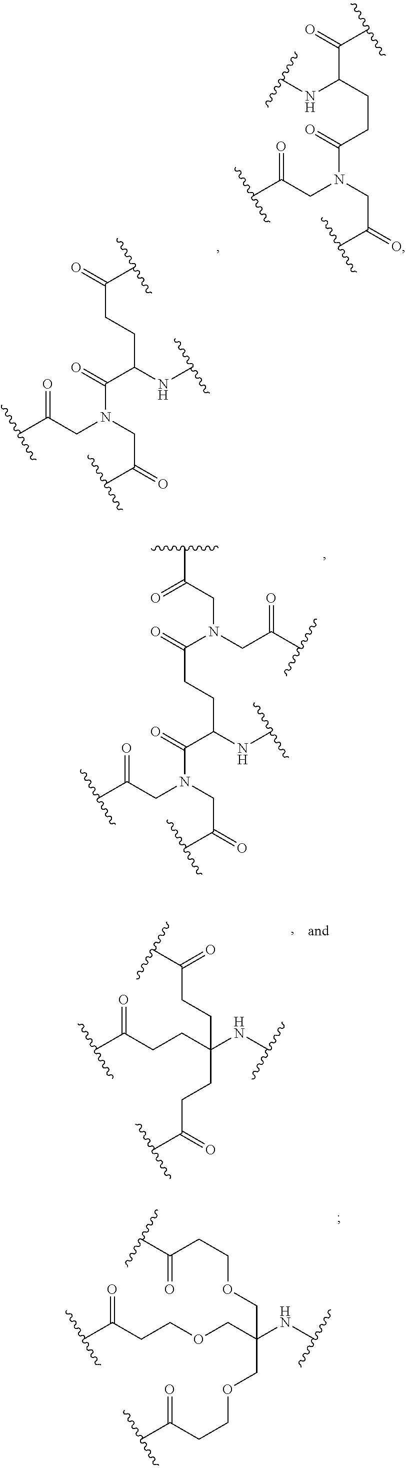 [SMILES: *NC(CCC(=O)N(CC(*)=O)CC(*)=O)C(*)=O ; *NC(CCC(*)=O)C(=O)N(CC(*)=O)CC(*)=O ; *NC(CCC(=O)N(CC(*)=O)CC(*)=O)C(=O)N(CC(*)=O)CC(*)=O ; *NC(CCC(*)=O)(CCC(*)=O)CCC(*)=O ; *NC(COCCC(*)=O)(COCCC(*)=O)COCCC(*)=O]

the linker is a structure selected from the group consisting of

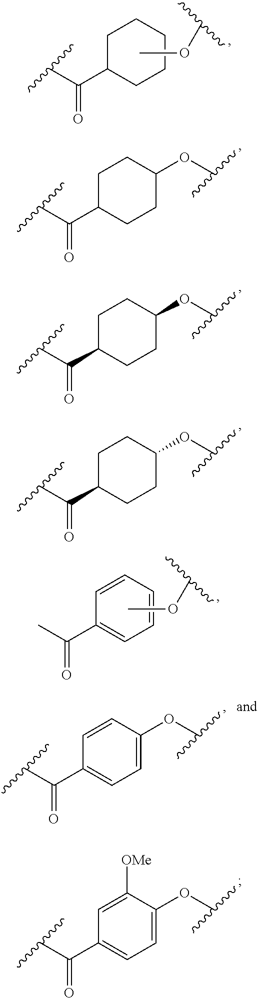

n is an integer from 1 to 4, as valency permits; and
[symbol] indicates the attachment point to the remainder of the compound.

Embodiment 3. The compound of Embodiment 2, wherein the tether is of the formula:

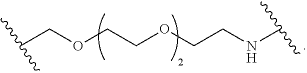

Embodiment 4. The compound of Embodiment 2, wherein the tether is of the formula:

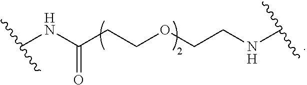

Embodiment 5. The compound of any one of Embodiments 2-4, wherein the branch point group is of the formula:

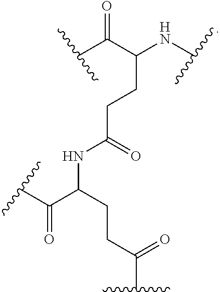 [SMILES: *NC(CCC(=O)NC(CCC(*)=O)C(*)=O)C(*)=O]

Embodiment 6. The compound of any one of Embodiments 2-5, wherein the branch point group is of the formula:

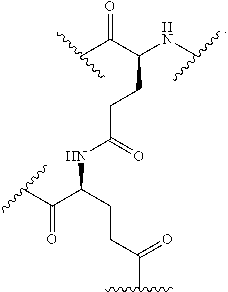 [SMILES: *N[C@@H](CCC(=O)N[C@@H](CCC(*)=O)C(*)=O)C(*)=O]

Embodiment 7. The compound of any one of Embodiments 2-4, wherein the branch point group is of the formula:

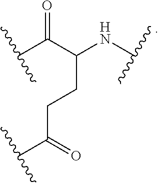 [SMILES: *NC(CCC(*)=O)C(*)=O]

Embodiment 8. The compound of any one of Embodiments 2-4, or 7, wherein the branch point group is of the formula:

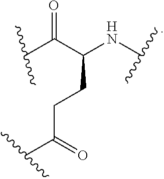 [SMILES: *N[C@@H](CCC(*)=O)C(*)=O]

Embodiment 9. The compound of any one of Embodiments 1-8, wherein the metabolically stabilized carbohydrate ligand comprises metabolically stabilized N-Acetylgalactosamine.

Embodiment 10. The compound of any one of Embodiments 1-9, wherein the metabolically stabilized carbohydrate is of the formula:

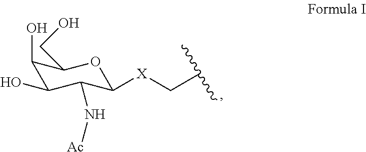

wherein X is CH₂ or S, and [symbol] indicates the point of connection to the remainder of the compound.

Embodiment 11. The compound of any one of Embodiments 1-9, wherein the metabolically stabilized carbohydrate is of the formula:

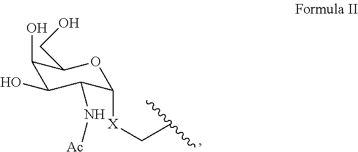

wherein X is CH₂ or S, and [symbol] indicates the point of connection to the remainder of the compound.

Embodiment 12. The compound of Embodiment 10 or 11, wherein X is CH₂.

Embodiment 13. The compound of Embodiment 10 or 11, wherein X is S.

Embodiment 14. The compound of any one of Embodiments 1-6, or 9-13, wherein the metabolically stabilized carbohydrate ligand comprises three metabolically stabilized N-Acetylgalactosamine moieties.

Embodiment 15. The compound of any one of Embodiments 2-6, or 9-13, wherein n is 3.

Embodiment 16. The compound of any one of Embodiments 2-15, wherein the linker is of the formula:

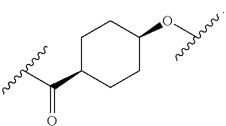 [SMILES: *O[C@H]1CC[C@@H](C(*)=O)CC1]

Embodiment 17. The compound of any one of Embodiments 1-16, wherein the metabolically stabilized carbohydrate ligand comprises a structure selected from the group consisting of:

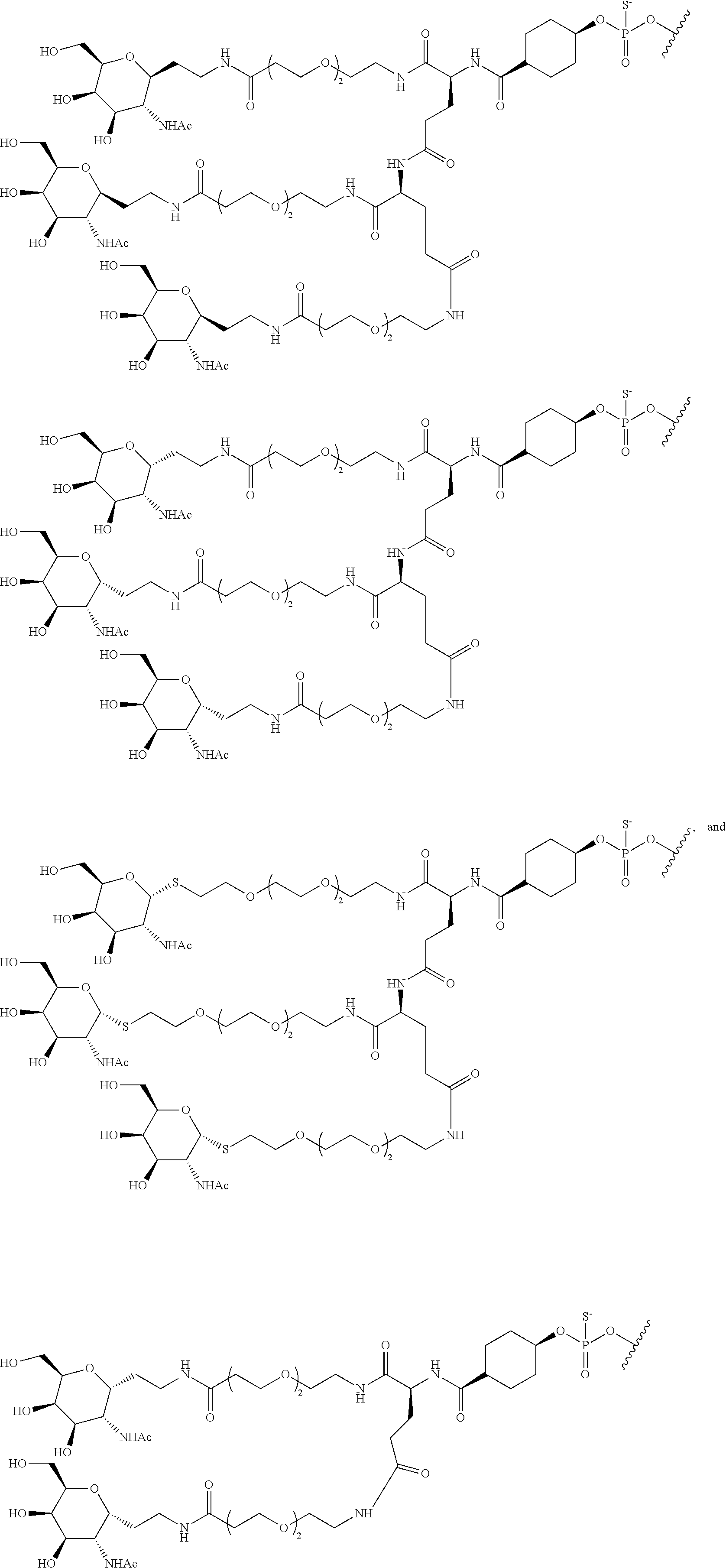

wherein: [symbol] indicates the point of connection to the remainder of the compound.

Embodiment 18. The compound of any one of Embodiments 1-17, wherein the oligonucleotide is a double-stranded RNAi agent comprising a sense strand and an antisense strand, wherein:
(i) the sense strand of the double-stranded RNAi agent comprises 19-23 nucleotides; and
(ii) the antisense strand of the double-stranded RNAi agent comprises 19-23 nucleotides.

Embodiment 19. The compound of any one of Embodiments 1-18, wherein the sense strand comprises 19-21 nucleotides and the antisense strand comprises 19-21 nucleotides.

Embodiment 20. The compound of any one of Embodiments 1-19, wherein the antisense strand of the double-stranded RNAi agent consists of 19 nucleotides.

Embodiment 21. The compound of any one of Embodiments 1-20, wherein the antisense strand is at least partially complementary to an mRNA sequence encoded by a gene expressed in a human hepatocyte.

Embodiment 22. The compound of any one of Embodiments 1-21, wherein the antisense strand is fully complementary to an mRNA sequence encoded by a gene expressed in a human hepatocyte.

Embodiment 23. The compound of any one of Embodiments 1-22, wherein the metabolically stabilized carbohydrate ligand is conjugated to the 3′ terminus of the sense strand.

Embodiment 24. The compound of any one of Embodiments 1-23, wherein the metabolically stabilized carbohydrate ligand is conjugated to the 5′ terminus of the sense strand.

Embodiment 25. The compound of any one of Embodiments 1-24, wherein an end cap is located at the 3′ end of the first sense strand, the 3′ end of the second sense strand, or both the 3′ end of the first sense strand and the second sense strand.

Embodiment 26. The compound of Embodiment 25, wherein the end cap is an inverted abasic moiety or NH2-C6.

Embodiment 27. A compound comprising a structure selected from the group consisting of:

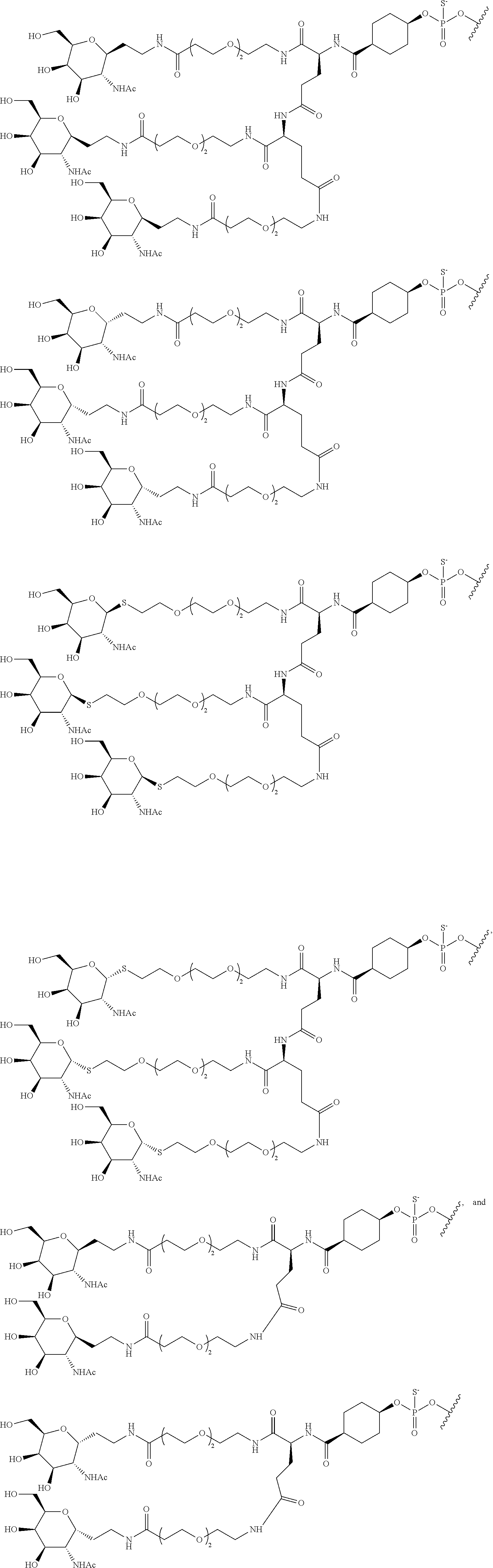

or a pharmaceutically acceptable salt thereof, wherein [symbol] indicates the point of connection to the remainder of the compound.

Embodiment 28. The compound of Embodiment 27, wherein the compound is of the formula:

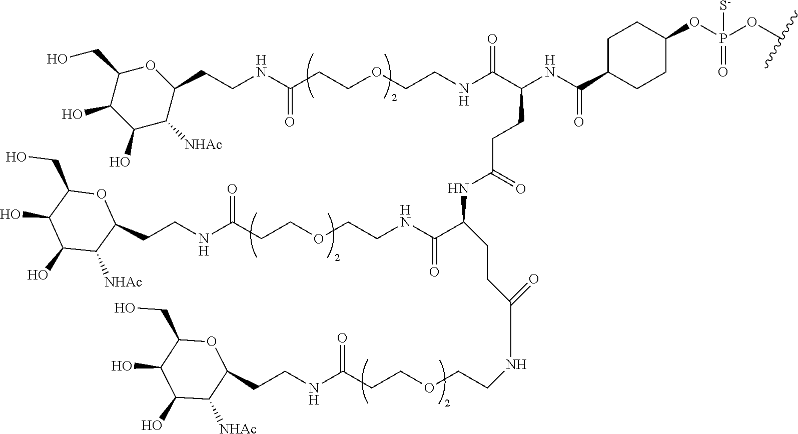

or a pharmaceutically acceptable salt thereof, wherein [symbol] indicates the point of connection to the remainder of the compound.

Embodiment 29. The compound of Embodiment 27, wherein the compound is of the formula:

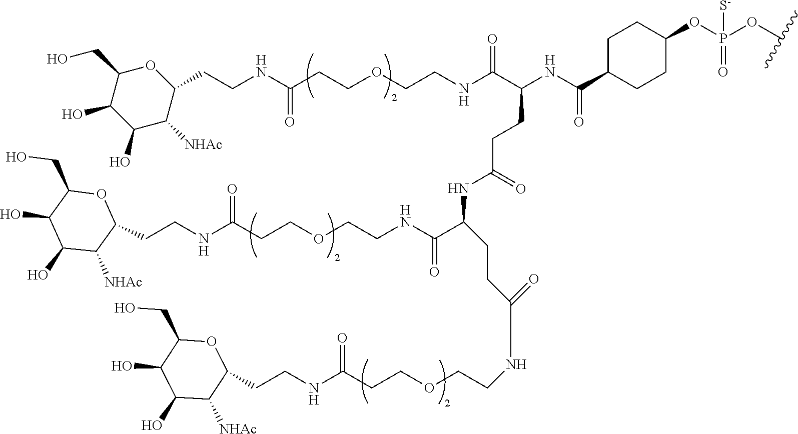

or a pharmaceutically acceptable salt thereof, wherein [symbol] indicates the point of connection to the remainder of the compound.

Embodiment 30. The compound of Embodiment 27, wherein the compound is of the formula:

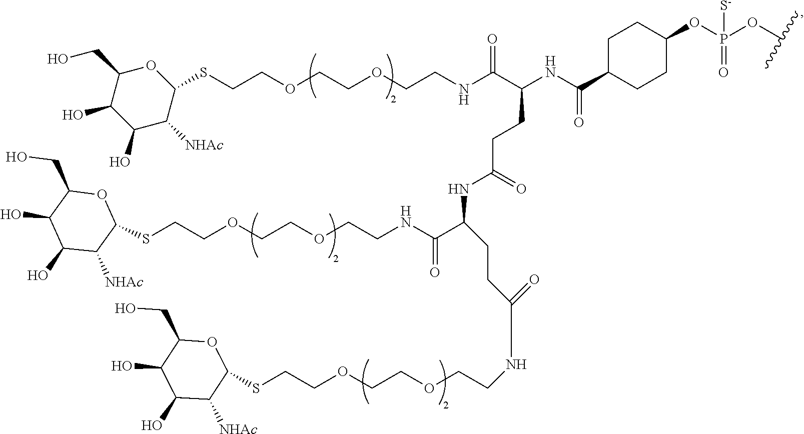

pharmaceutically acceptable salt thereof, wherein [symbol] indicates the point of connection to the remainder of the compound.

Embodiment 31. The compound of Embodiment 27, wherein the compound is of the formula:

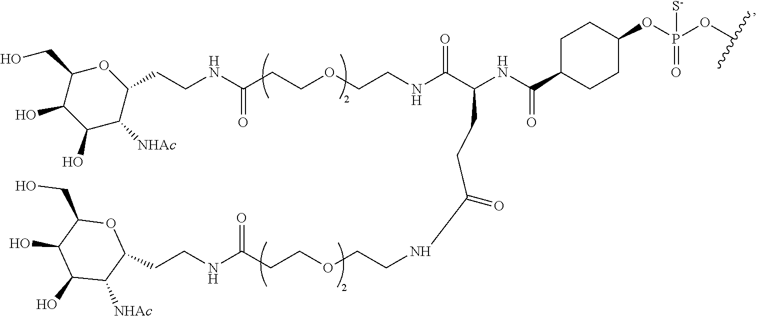

or a pharmaceutically acceptable salt thereof, wherein [symbol] indicates the point of connection to the remainder of the compound.

Embodiment 32. The compound of any one of Embodiments 27-31, wherein the remainder of the compound comprises an oligonucleotide strand that is 12-49 nucleotides in length.

Embodiment 33. The compound of Embodiment 32, wherein the oligonucleotide is a double-stranded RNAi agent comprising a sense strand and an antisense strand, wherein:
(i) the sense strand of the double-stranded RNAi agent comprises 19-23 nucleotides; and
(ii) the antisense strand of the double-stranded RNAi agent comprises 19-23 nucleotides.

Embodiment 34. The compound of Embodiment 33, wherein the sense strand comprises 19-21 nucleotides and the antisense strand comprises 19-21 nucleotides.

Embodiment 35. The compound of Embodiment 33 or 34, wherein the antisense strand of the double-stranded RNAi agent consists of 19 nucleotides.

Embodiment 36. The compound of any one of Embodiments 33-35, wherein the antisense strand is at least partially complementary to the mRNA encoded by a gene expressed in a human hepatocyte.

Embodiment 37. The compound of any one of Embodiments 33-36, wherein the antisense strand is fully complementary to the mRNA encoded by a gene expressed in a human hepatocyte.

Embodiment 38. The compound of any one of Embodiments 33-37, wherein an end cap is located at the 3′ end of the sense strand.

Embodiment 39. The compound of any one of Embodiments 33-38, wherein an end cap is located at the 5′ end of the sense strand.

Embodiment 40. A pharmaceutical composition comprising a compound of any one of Embodiments 1-39 and a pharmaceutically acceptable excipient.

Embodiment 41. A method of inhibiting expression of a gene, comprising administering to a subject in need thereof a compound of any one of Embodiments 1-39.

### EXAMPLES

The following examples are not limiting and are intended to illustrate certain embodiments disclosed herein.

### Example 1. Synthesis of RNAi Agents and Multimeric RNAi Agents Conjugates

The following describes the general procedures for the syntheses of certain RNAi agents, and conjugates thereof, including the RNAi conjugates that are illustrated in the non-limiting Examples set forth herein.

Synthesis of RNAi Agents. RNAi agents can be synthesized using methods generally known in the art. For the synthesis of the RNAi agents illustrated in the Examples set forth herein, the sense and antisense strands of the RNAi agents were synthesized according to phosphoramidite technology on solid phase used in oligonucleotide synthesis. Depending on the scale, a MerMade96E® (Bioautomation), a MerMadel2® (Bioautomation), or an Oligopilot 100 (GE Healthcare) was used. Syntheses were performed on a solid support made of controlled pore glass (CPG, 500 Å or 600 Å, obtained from Prime Synthesis, Aston, PA, USA) or polystyrene (obtained from Kinovate, Oceanside, CA, USA). All RNA and 2′-modified RNA phosphoramidites were purchased from Thermo Fisher Scientific (Milwaukee, WI, USA), ChemGenes (Wilmington, MA, USA), or Hongene Biotech (Morrisville, NC, USA). Specifically, the 2′-O-methyl phosphoramidites that were used include the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites and 2′-O-propargyl phosphoramidites carried the same protecting groups as the 2′-O-methyl phosphoramidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes. The following UNA phosphoramidites that were used included the following: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile or a 200 mM solution of xanthane hydride (TCI America, Portland, OR, USA) in pyridine was employed.

TFA aminolink phosphoramidites were also commercially purchased (ThermoFisher) to introduce the (NH2-C6) reactive group linkers. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ 0-Me), and 60 sec (2′ F). Trialkyne-containing phosphoramidites were synthesized to introduce the respective (TriAlk #) linkers. When used in connection with the RNAi agents presented in certain Examples herein, trialkyne-containing phosphoramidites were dissolved in anhydrous dichloromethane or anhydrous acetonitrile (50 mM), while all other amidites were dissolved in anhydrous acetonitrile (50 mM), and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ O-Me), and 60 sec (2′ F).

For some RNAi agents, a linker, such as a C6-SS-C6 or a 6-SS-6 group, C6-SS(Me)-C5 was introduced at the 3′ terminal end of the sense strand. Pre-loaded resin was commercially acquired with the respective linker. Alternatively, for some sense strands, a dT resin was used and the respectively linker was then added via standard phosphoramidite synthesis.

Cleavage and deprotection of support bound oligomer. After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

Purification. Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G25 fine with a running buffer of 100 mM ammonium bicarbonate, pH 6.7 and 20% Acetonitrile or filtered water.

Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor and the dilution factor to determine the duplex concentration. The conversion factor used was either 0.037 mg/(mL·cm) or was calculated from an experimentally determined extinction coefficient.

### Example 2. Synthesis of Targeting Ligands

### Synthesis of NAG52 (Compound 9)

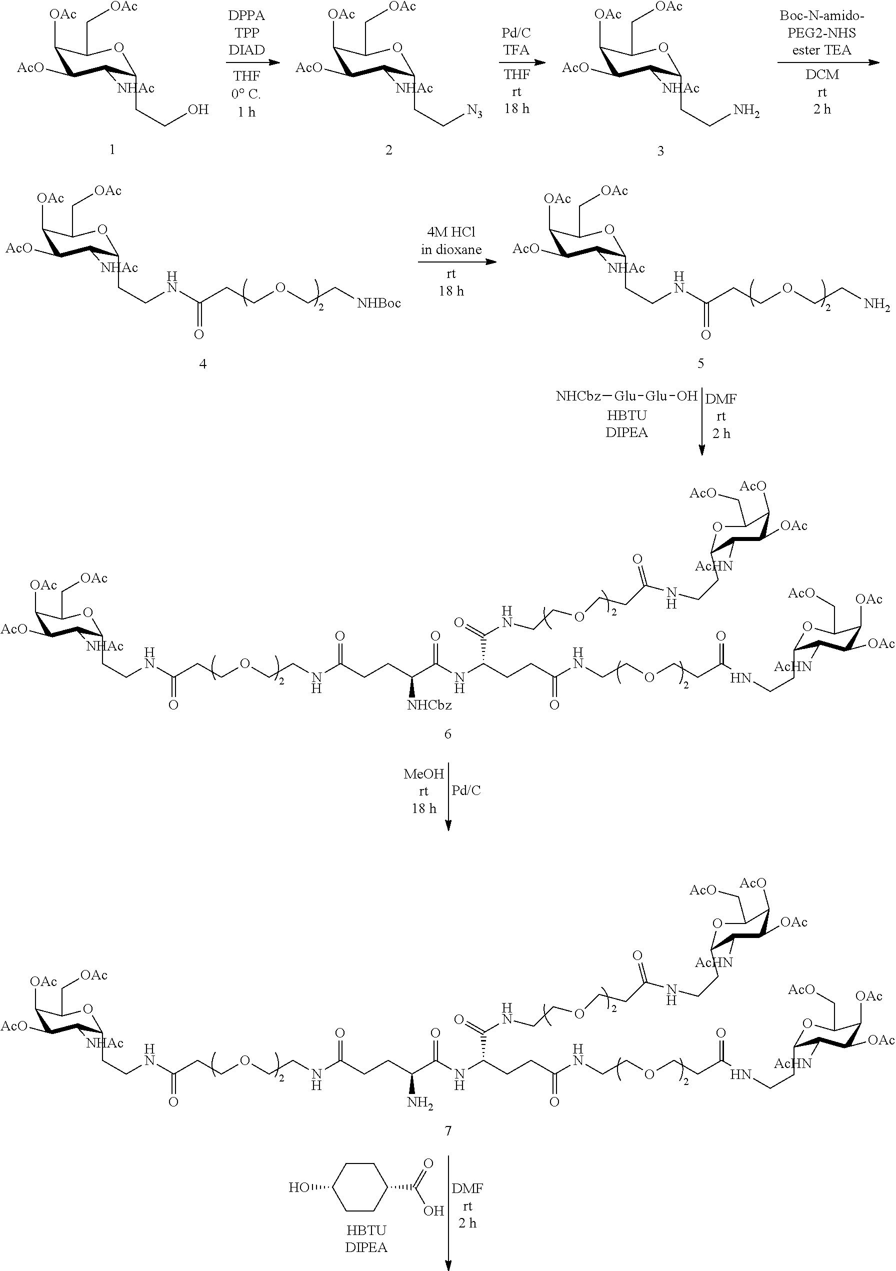

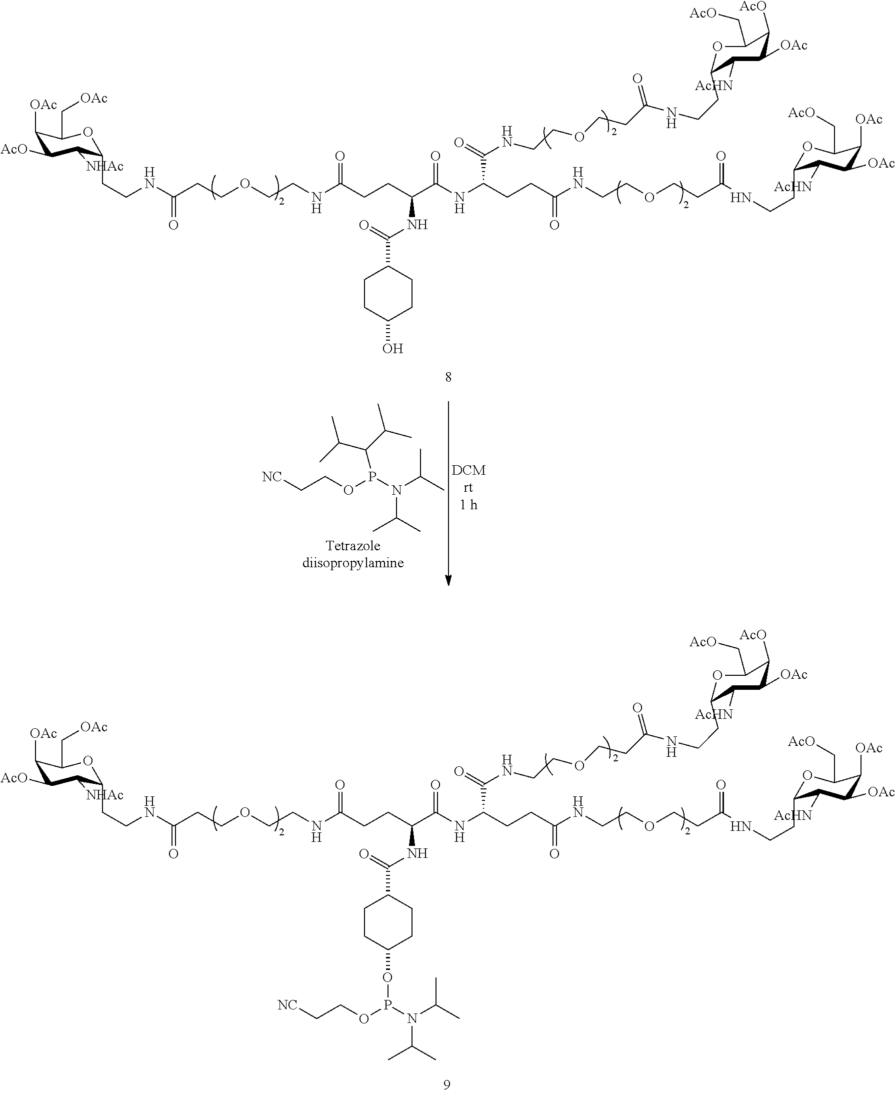

Compounds 1, 2 and 3 were each synthesized in accordance with previously published procedures (see, e.g., U.S. Patent Application Publication No. 2002/0107224 A1), and Cbz-NH-Glu-Glu-OH:

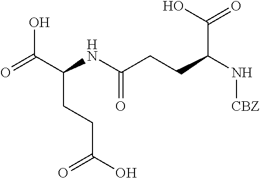

was synthesized in accordance with the procedure described in International Patent Application Publication No: WO 2017/156012 to Arrowhead Pharmaceuticals, Inc.), the contents of those references are incorporated by reference as if fully set forth herein. More specifically, 1 was synthesized from the following synthetic route:

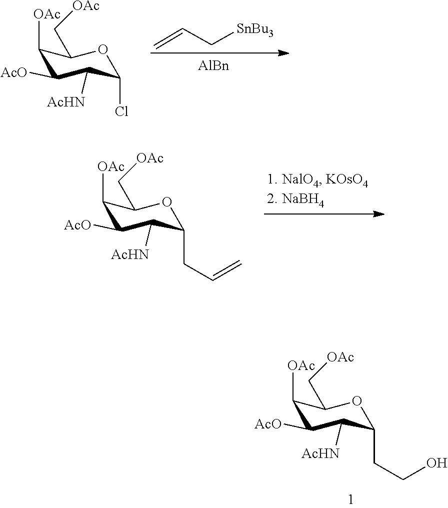

### Synthesis of 5

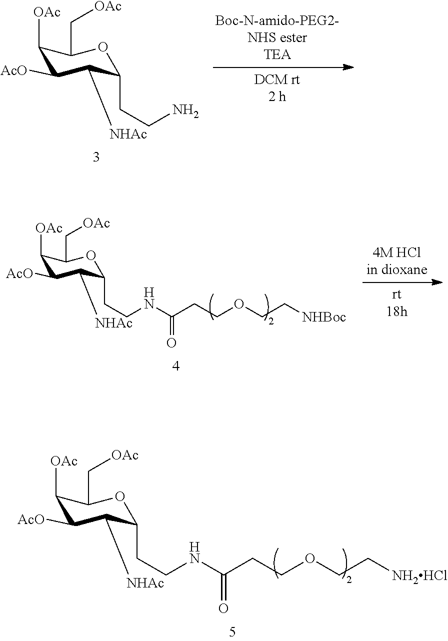

3 (4.61 g, 12.3 mmol) and Boc-N-amido-PEG2-NHS ester (CAS 2183440-73-3, 4.61 g, 12.3 mmol) were dissolved in anhydrous DCM (100 mL) followed by addition of triethylamine (3.4 mL, 24.6 mmol). The reaction mixture was stirred at room temperature (rt) for 2 h, the solution was concentrated down to 30 mL under reduced pressure and diluted with chloroform (300 mL). The resulting solution was first washed with brine/citric acid (1:1, 30 mL) and then with brine/saturated bicarbonate solution (1:1, 30 mL). The organic layer was dried over Na₂SO₄, concentrated under reduced pressure, and purified on a silica column (100% DCM to 20% MeOH in DCM). Fractions containing the desired product 4 were combined, the solvent was removed under reduced pressure and the resulting foaming residue was redissolved in 4M HCl in 1,4-dioxane (100 mL). The reaction mixture was stirred at rt for 1 h, and the solvent was removed under reduced pressure. The resulting residue was suspended in toluene and dried under reduced pressure giving the desired product 5 as an HCl salt (5.30 g, 9.30 mmol, 76% yield). Calculated MW 533.26 Found ESI MS⁺ m/z=534.23 [M+H⁺].

### Synthesis of 7

5 (5.30 g, 9.94 mmol) was dissolved in anhydrous DMF followed by addition of DIPEA (4.4 mmol, 45.97 mmol). Z-BisGlu (1.12 g, 2.73 mmol) and TBTU (3.03 g, 7.99 mmol) was added upon vigorous stirring. The solution turned light maroon, darkening over time. The reaction mixture was left stirring at rt for 2 h when no unreacted starting material could be detected by LC-MS. The solvent was removed by co-evaporation with toluene, and the residue was redissolved in chloroform (400 mL). The resulting solution was first washed with brine/water (1:1, 60 mL) and then with brine/bicarbonate solution (1:1, 60 mL). The organic layer was dried over Na₂SO₄, concentrated under reduced pressure, and purified on a silica column (100% DCM to 20% MeOH in DCM). Fractions containing the desired product 6 were combined, the solvent was removed under reduced pressure and the resulting foaming residue was redissolved in methanol (200 mL). Pd/C (0.70 g) was added to the solution, the suspension was hydrogenated under 1 atm overnight. The reaction mixture was stirred under hydrogen at rt overnight. The solution was filtered through a celite pad and concentrated under reduced pressure to yield the desired product 7 that was used as is in the next step (3.80 g, 2.09 mmol, 77% yield). Calculated MW 1821.84 Found ESI MS⁺ m/z=912.13 [M+2H⁺].

### Synthesis of 8

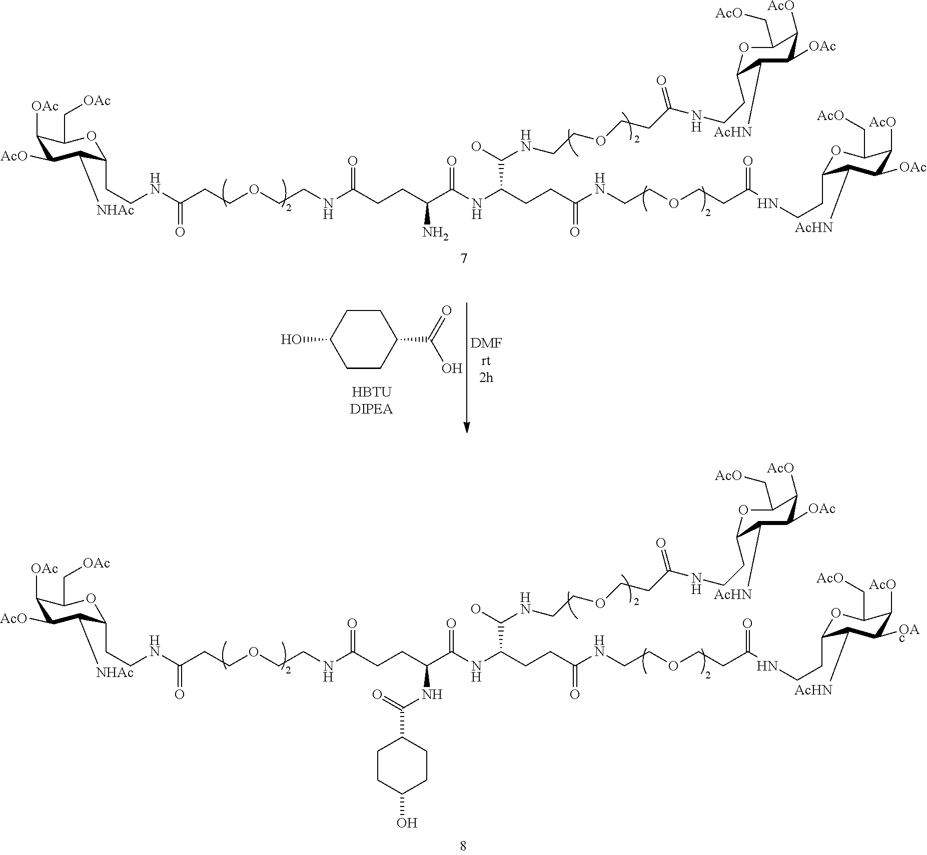

7 (3.80 g, 2.09 mmol) was dissolved in anhydrous DMF (30 mL) and was slowly added to a solution containing 4-hydroxycyclohexane carboxylic acid (0.35 g, 2.43 mmol), TBTU (0.82 g, 2.16 mmol) and DIPEA (1.12 mL, 6.44 mmol) in anhydrous DMF (30 mL). The reaction mixture was stirred at rt for 2 h. The solvent was removed by co-evaporation with toluene, and the residue was redissolved in chloroform (300 mL). The solution was washed with brine/5% citric acid (1:1, 30 mL), dried over Na₂SO₄ and concentrated under reduced pressure and purified on a silica column (100% DCM to 30% MeOH in DCM) to yield the desired product 8 (2.40 g, 1.23 mmol, 59% yield). Calculated MW 1947.90 Found ESI MS⁺ m/z=975.46 [M+2H⁺].

### Synthesis of 9

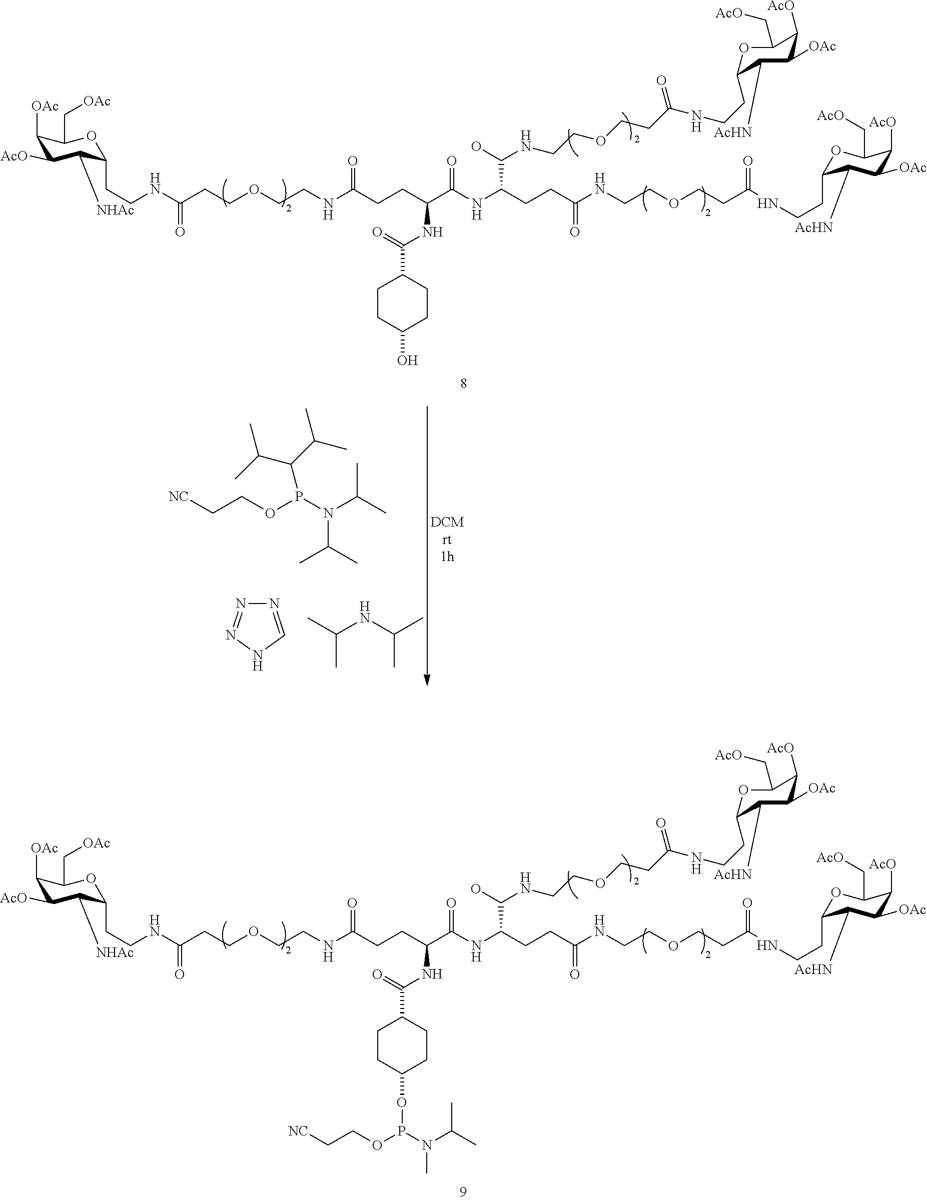

Compound 8 (2.40 g, 1.23 mmol) was thoroughly dried by co-evaporating DCM with toluene and dried in vacuo for 30 min. A round-bottom flask was charged with a stir bar and pre-treated molecular sieves, and was purged with nitrogen. The flask was filled with DCM (100 mL), and the molecular sieves were gently stirred for 10 min. Diisopropylammonium tetrazolide (1.40 g, 8.19 mmol) was added to the solution, and the reaction mixture was stirred for another 30 min. 2-Cyanoethyl N,N,N′,N′-tetraisopropylphosphorodiamidite (0.55 mL, 1.73 mmol) was added, and the reaction mixture was stirred for 1 h at rt when no unreacted starting material could be detected by LC-MS. The solution was filtered through a celite pad to remove molecular sieves and diluted with saturated bicarbonate solution (100 mL) upon stirring. After 15 min, the organic layer was separated, and the aqueous layer was extracted with chloroform (2×200 mL). The organic fractions were combined, dried over Na₂SO₄, concentrated under reduced pressure and purified on a silica column (100% DCM (+0.1% triethylamine) to 10% MeOH in DCM (+0.1% triethylamine)). The fractions containing the desired product 9 were combined, concentrated under reduced pressure, and the product was co-evaporated twice with toluene to remove any residual triethylamine to give the desired product as an off-white solid (2.4 g, 1.16 mmol, 94% yield).

### Compound 9 Characterization:

¹H NMR (DMSO-d6): 1.14 d (12H), 1.44 m (7H), 1.62-1.90 m (11H), 1.80 s (9H), 1.94 s (9H), 2.00 s (9H), 2.07 s (9H), 2.03-2.16 m (4H), 2.20-2.31 m (6H), 2.76 t (2H), 2.88-2.98 m (3H), 3.12-3.23 m (10H), 3.34-3.42 m (6H), 3.46 s (12H), 3.57 t (8H), 3.62-3.76 m (2H), 3.98-4.20 m (15H), 4.20-4.30 m (3H), 4.96 dd (3H), 5.28, d (3H), 7.56-8.00 m (8H), 8.12 d (3H).

³¹P NMR (DMSO-d6): 145.84, 146.01

### Synthesis of NAG42 (Compound 9B)

The synthesis of NAG42 follows the same synthetic route as NAG52 described above, with the only change being that it employs a beta anomeric stabilized linkage instead of an alpha anomeric linkage. More specifically, compound 1B having a beta anomeric linkage can be synthesized as follows:

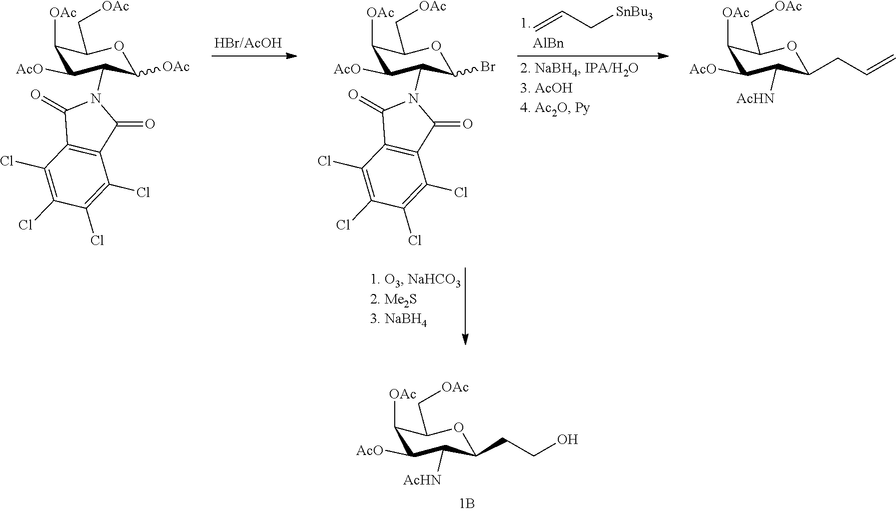

The remaining synthesis follows what is described above for the synthesis of NAG52, with 1B replacing 1, and resulting in compound 9B:

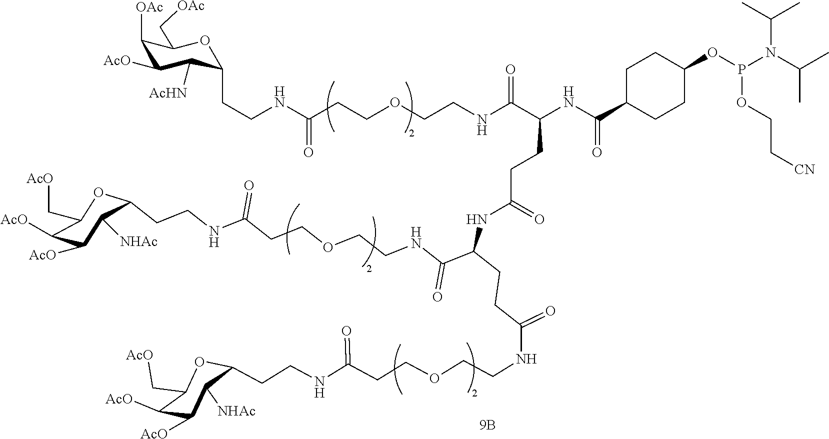

### Compound 9B Characterization:

¹H NMR (DMSO-d6): 1.14 d (12H), 1.36-1.54 m (7H), 1.60-1.86 m (11H), 1.79 s (9H), 1.89 s (9H), 1.99 s (9H), 2.10 s (9H), 2-02-2.16 m (4H), 2.24-2.30 m (6H), 2.76 t (2H), 2.98-3.08 m (3H), 3.12-3.24 m (10H), 3.30-3.42 m (8H), 3.47 s (12H), 3.58 t (8H), 3.62-3.76 m (2H), 3.80-4.06 m (14H), 4.10-4.20 m (2H), 4.88 dd (3H), 5.26 d (3H), 7.55-8.00 m (11H).

³¹P NMR (DMSO-d6): 145.84, 145.89

### Synthesis of NAG1008 Phosphoramidite

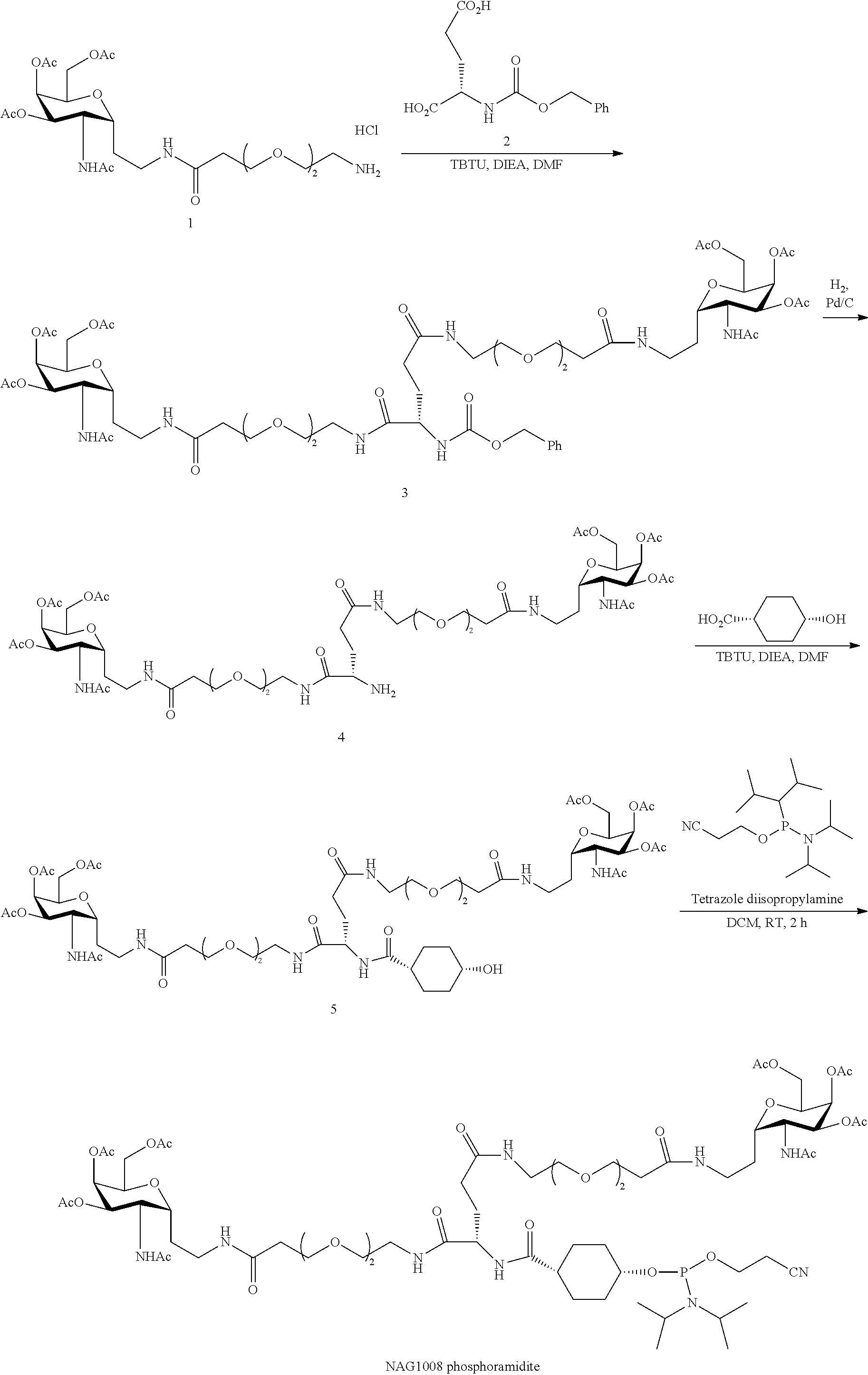

### Synthesis of 3.

Hydrochloride salt of α-C-Nag-Peg₂-amine 1 (3.864 g, 6.79 mmol), was taken in anh. DMF (250 mL), glutamic acid derivative 2 (734 mg, 2.61 mmol) and DIEA (4 mL, 23 mmol), and TBTU (1.860 g, 5.8 mmol) were added. The pH was checked to confirm that it was basic. The reaction was stirred for 1.5 h, all volatiles were removed in vacuo at 40° C., toluene was evaporated ×2 to remove residual DMF. The product was dissolved in CHCl₃, washed twice with 10% aq. NaCl, and sat. aq. NaHCO₃, and dried (Na₂SO₄). Combiflash purification was performed using 80 g column, eluent A=DCM; B=20% MeOH in DCM, 0-60% in 60 min. Yield 2.915 g, 81%. Calculated MW 1311.59 Found ESI MS⁺ m/z=1312.42 [M+H⁺].

### Synthesis of 4.

Product 3 (2.910 g, 2.222 mmol) was hydrogenated in with 10% Pd/C (330 mg) in MeOH (60 mL) for 16 h with hydrogen balloon. The product was filtered via celite, concentrated and dried in vacuo. The product was additionally dried by evaporation of toluene and used directly in the next step. Calculated MW 1177.55 Found ESI MS⁺ m/z=1179.02 [M+H⁺].

### Synthesis of 5.

Cis-4-hydroxycyclohexecarboxylic acid (368 mg, 2.56 mmol) was treated with TBTU (855 mg, 2.66 mmol) and DIEA (1.16 mL, 6.67 mmol) in anh. DMF (20 mL) for 3 min.

Compound 4 from the previous step (2.222 mmol) was dissolved in anh. DMF (40 mL) and added into the solution with activated acid. Following 1.5 h of stirring, DMF was removed in vacuo at 40° C., toluene was evaporated twice to get rid of residual DMF. The residue was taken in chloroform (150 mL), washed twice with 10% aq. NaCl, aq. NaHCO₃, dried (Na₂SO₄). Yield 1.89 g (65%). MS: calculated MW 1303.62 Found ESI MS⁺ m/z=1304.42 [M+H⁺]. Crude product was used directly in the next step.

### Synthesis of NAG1008 Phosphoramidite.

Crude precursor 5 (1.449 mmol, 1.89 g) was dried by 2 evaporations of toluene and redissolved in anh. DCM (60 mL). NN,-Diisopropylammonium tetrazolide (348 mg, 2 mmol) and molecular sieves (100 mg) were added, the mixture was stirred for 45 min. 2-Cyanoethyl N,N,N′,N′-tetraisopropylphosphorodiamidite (611 mg, 2 mmol) was added, the reaction was stirred for 4 h, filtered, and stirred with cold NaHCO₃ (vv 10%) in DCM for 15 min. The aq. layer was separated, extracted with CHCl₃ (×2), dried (Na₂SO₄), filtered and concentrated. The crude (2.02 g) was purified on CombiFlash. Column 24 g, liquid load. Eluent: A=DCM; B=MeOH 20% in DCM, 0-25%, 30 min. Yield 1.357 g, (62%). MS: calculated MW 1503.72 Found ESI MS⁻ m/z=1502.42 [M−H⁺]. NMR P-31, (DMSO, d6): 144.913, 144.944.

### Synthesis of NAG55 Phosphoramidite

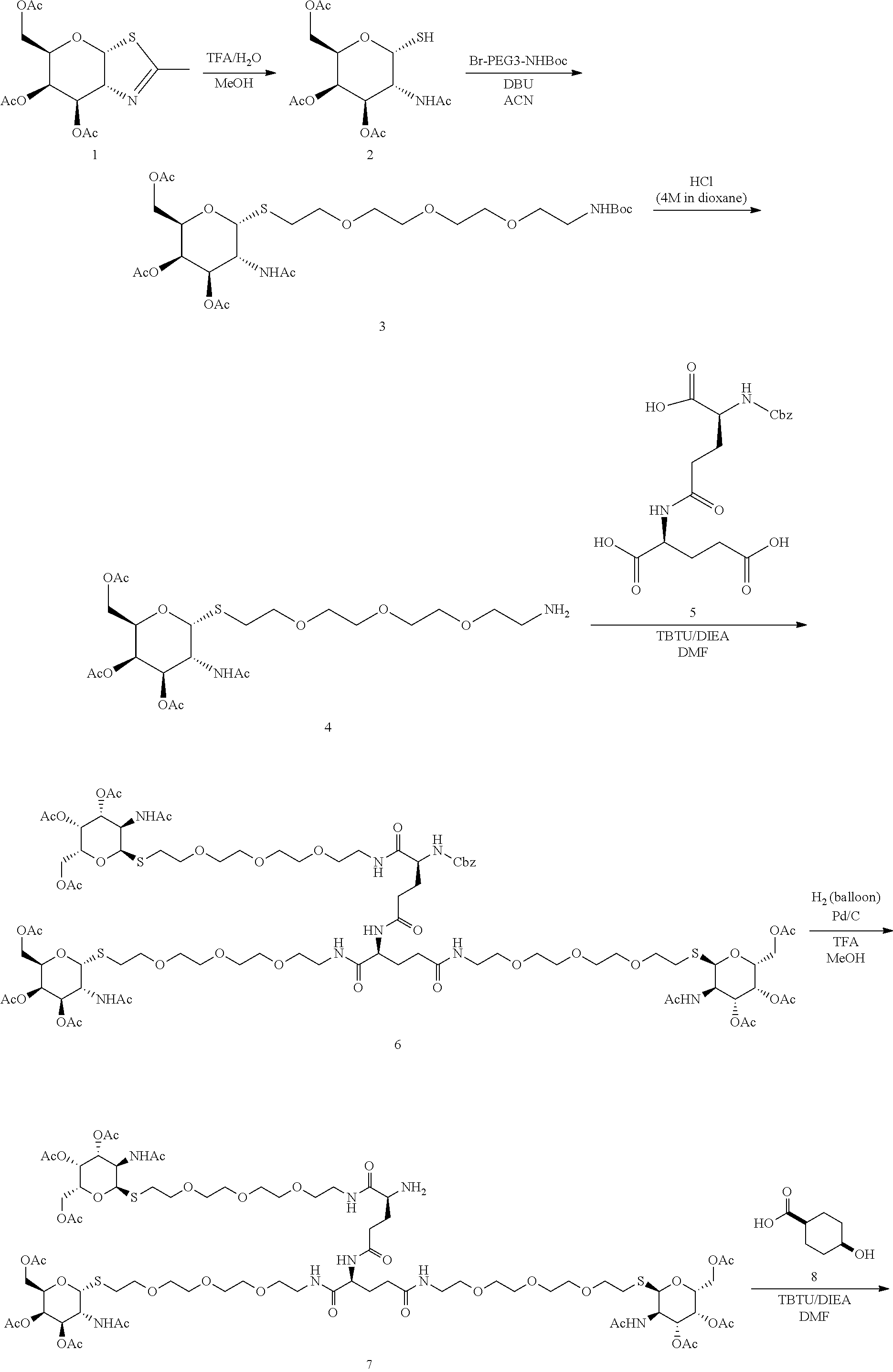

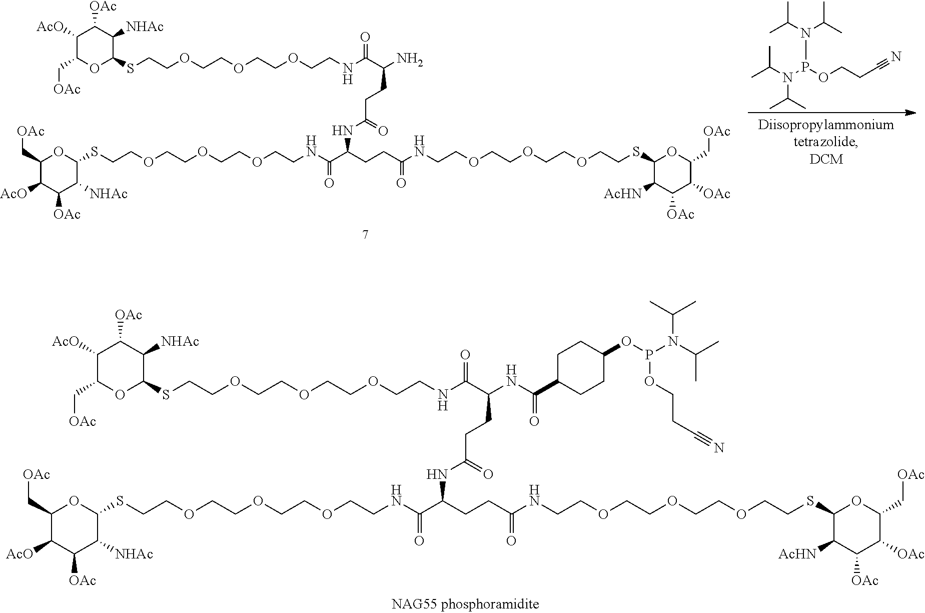

### Synthesis of Compound 2:

A solution of compound 1 (1 equiv.) in MeOH (12 volumes) was cooled to 0° C. To this stirring solution, TFA (0.8 volumes) and water (0.8 volumes) was added. The solution was allowed to warm to room temperature and stirred for 2 hours. The reaction concentrated and the crude of compound 2 was used on the next step.

### Synthesis of Compound 3:

Crude of compound 2 (1 equiv.) dissolved in acetonitrile (8 volumes) under nitrogen atmosphere and cooled in acetone/dry ice bath. DBU (1.5 equiv.) and Br-PEG3-NHBoc (1.05 equiv.) added, and reaction allowed to warm to room temperature and stirred overnight.

Solvent evaporated and the crude purified by column chromatography to give compound 3.

### Synthesis of Compound 4:

Compound 3 (1 equiv.) was added to a flask and 3.7 volumes of HCl solution (4M in 1,4-dioxane) was added and the reaction stirred at room temperature for 2.5 hours. The solution concentrated and the crude of compound 4 was used on the next step.

### Synthesis of Compound 6:

Crude of compound 4 (3.7 equiv.) and compound 5 (1 equiv.) dissolved in DMF (50 volumes). DIEA (15 equiv.) and TBTU (3.5 equiv.) added to this solution and stirred at room temperature for 2 hours. The reaction concentrated, then crude dissolved in chloroform (3.5 volumes) and washed with water/brine (1:1) and water/saturated NaHCO₃ (1:1) solutions. The organic layer dried on Na₂SO₄ and concentrated. The crude was purified by column chromatography to give compound 6.

### Synthesis of Compound 7:

A solution of compound 6 (1 equiv.) and TFA (10 equiv.) was stirred in MeOH in presence of Pd/C (50 wt %) under H₂ atmosphere for 2 hours at room temperature. Solution was filtered and concentrated to give compound 7.

### Synthesis of Compound 9:

Compound 7 dissolved in DCM (32 volumes) under nitrogen atmosphere and cooled to 0° C. In another flask, compound 8 (1.1 equiv.), TBTU (1.1 equiv.) and DIEA (3.5 equiv.) stirred in DCM (20 volumes) for 15 minutes, then added to the compound 7 solution and stirred at room temperature. After 3 hours, again in another flask, compound 8 (1.1 equiv.), TBTU (1.1 equiv.) and DIEA (3.5 equiv.) was stirred in DCM for 10 minutes and added to the main reaction flask. After another 15 minutes stirring, saturated NH₄Cl added and extracted with DCM (3 times). Combined organic layers were washed with saturated solution of NaHCO₃ and brine. Then dried on Na₂SO₄, and concentrated. The crude purified by column chromatography to give compound 9.

### Synthesis of NAG55 Phosphoramidite:

Compound 9 dissolved in DCM (15 volumes) under nitrogen atmosphere. 2-cyanoethyl N,N,N′,N′-tetraisopropylphosphorodiamidite (2.7 equiv.) and diisopropylammonium tetrazolide (0.7 equiv.) was added and stirred for 2 hours. Then, more of 2-cyanoethyl N,N,N′,N′-tetraisopropylphosphorodiamidite (0.5 equiv.) and diisopropylammonium tetrazolide (0.3 equiv.) was added and stirred for another 1.5 hours. Reaction cooled to 0° C. and washed with saturated solution of NaHCO₃. Organic layer dried on Na₂SO₄, and concentrated. The crude purified by column chromatography to give NAG55 phosphoramidite.

### Example 3. Conjugation of Linkers and Targeting Ligands to RNAi Agents

### A. Conjugation of Activated Ester Linkers

One potential method for conjugation of linkers is by the coupling of activated esters. In some embodiments, the following procedures may be used to conjugate linking groups having terminal propargyl groups to an RNAi agent with an amine-functionalized sense strand, such as C6-NH2, NH2-C6, or (NH2-C6)s, as shown in Table 2, above. An annealed RNAi Agent dried by lyophilization is dissolved in DMSO and 10% water (v/v %) at 25 mg/mL. Then 50-100 equivalents of TEA and 3 equivalents of activated ester linker are added to the solution. The solution is allowed to react for 1-2 hours, while monitored by RP-HPLC-MS (mobile phase A 100 mM HFIP, 14 mM TEA; mobile phase B: acetonitrile on an XBridge C18 column, Waters Corp.)

The product can then be precipitated by adding 12 mL acetonitrile and 0.4 mL PBS and centrifuging the solid to a pellet. The pellet is then re-dissolved in 0.4 mL of 1×PBS and 12 mL of acetonitrile. The resulting pellet is dried on high vacuum for one hour.

### B. Conjugation of Targeting Ligands to Propargyl Linkers

Similarly, another acceptable method to couple targeting ligands of the disclosed compounds herein is through their conjugation to propargyl linkers. In some embodiments, either prior to or after annealing, a 5′ or 3′ tridentate alkyne functionalized sense strand can be conjugated to the NAG ligand. The following describes one possible method for the conjugation of α/ρ-anomeric metabolically stabilized NAG to an annealed duplex: Stock solutions of 0.5M Tris(3-hydroxypropyltriazolylmethyl)amine (THPTA), 0.5M of Cu(II) sulfate pentahydrate (Cu(II)SO₄·5H₂O) and 2M solution of sodium ascorbate are prepared in deionized water. A 75 mg/mL solution in DMSO of NAG ligand azide is made. In a 1.5 mL centrifuge tube containing tri-alkyne functionalized duplex (3 mg, 75 μL, 40 mg/mL in deionized water, approximately 15,000 g/mol), 25 μL of 1M Hepes pH 8.5 buffer is added. After vortexing, 35 μL of DMSO is added and the solution is vortexed. The ligand can then be added to the reaction (e.g., 6 eq/duplex, 2 eq/alkyne, approximately 15 μL) and the solution is vortexed. Using pH paper, pH is checked and confirmed to be pH approximately 8. In a separate 1.5 mL centrifuge tube, 50 μL of 0.5M THPTA is mixed with 10 uL of 0.5M Cu(II)SO₄·5H₂O, vortexed, and incubated at room temp for 5 min. After 5 min, THPTA/Cu solution (7.2 μL, 6 eq 5:1 THPTA:Cu) is added to the reaction vial, and vortexed. Immediately afterwards, 2M ascorbate (5 μL, 50 eq per duplex, 16.7 per alkyne) is added to the reaction vial and vortexed. Once the reaction was complete (typically complete in 0.5-1 h), the reaction mixture is immediately purified by non-denaturing anion exchange chromatography.

### C. Conjugation of Targeting Ligands to Amine-Functionalized Sense Strand

In some embodiments, the following procedure may be used to conjugate an activated ester-functionalized targeting ligand such as a metabolically stabilized carbohydrate ligand to an amine functionalized RNAi agent comprising an amine, such as C6-NH2, NH2-C6, or (NH2-C6)s, as shown in Table 2: An annealed, lyophilized RNAi agent is dissolved in DMSO and 10% water (v/v %) at 25 mg/mL. Then 50-100 equivalents TEA and three equivalents of activated ester targeting ligand are added to the mixture. The reaction mixture is allowed to stir for 1-2 hours while monitored by RP-HPLC-MS (mobile phase A: 100 mM HFIP, 14 mM TEA; mobile phase B: Acetonitrile; column: XBridge C18). After the reaction mixture is complete, 12 mL of acetonitrile was added followed by 0.4 mL of PBS and then the mixture is centrifuged. The solid pellet is collected and dissolved in 0.4 mL of 1×PBS and then 12 mL of acetonitrile is added. The resulting pellet is collected and dried under vacuum for 1 hour.

### D. Addition of Targeting Ligands by Phosphoramidite Synthesis or On Resin.

Other acceptable methods to couple targeting ligands are to prepare the desired ligand as a phosphoramidite compound, which may be added to the 5′ end of the strand using standard solid phase synthesis, or to prepare the targeting ligand on resin which can be placed at the 3′ end of the strand after cleavage, again using standard solid phase oligonucleotide synthesis.

### Example 4. In Vivo Administration of Metabolically Stabilized RNAi Agent Conjugates in Cynomolgus Monkeys

Multimeric (dimer) RNAi agent conjugates that included metabolically stabilized NAG targeting ligands were evaluated for gene silencing activity in cynomolgus macaque (Macaca fascicularis) primates (referred to herein as “cynos”). Each of the multimeric RNAi agent conjugates evaluated included one RNAi agent of the conjugate having sufficient complementarity with the mouse Angiopoietin-like 3 (ANGPTL3) gene transcript, and a second RNAi agent having sufficient complementarity with the mouse Factor 12 (FXII) gene transcript. As discussed in more detail below, both FXII and ANGPTL3 gene expression levels were assessed.

At day 1, male cynos were given a single subcutaneous administration of 0.3 mL/kg animal weight (20 mg/mL concentration) containing 6.0 mg/kg (mpk) of a multimeric RNAi agent conjugate formulated in isotonic saline, or 3.0 mg/kg of two separate RNAi conjugates, according to the following Table 3.

TABLE 3
Dosing Groups of Example 4

| Group | RNAi Agent and Dose | Platform Design | Dosing Regimen |
|---|---|---|---|
| 1 | 3.0 mg/kg AD14219 | Two monomeric | Single injection |
| | (AC912316) + | RNAi-agent | on day 1 |
| | 3.0 mg/kg AD14220 | conjugates | |
| | (AC912317) | administered | |
| | (two monomeric RNAi- | separately | |
| | agent conjugates | | |
| | administered in a 1:1 | | |
| | mix) | | |
| 2 | 6.0 mg/kg AD14216 | Multimeric | Single injection |
| | (AC912313) | (Dimer) | on day 1 |
| 3 | 6.0 mg/kg AD14217 | Multimeric | Single injection |
| | (AC912314) | (Dimer) | on day 1 |
| 4 | 6.0 mg/kg AD14218 | Multimeric | Single injection |
| | (AC004352) | (Dimer) | on day 1 |

Each of the multimeric RNAi agent conjugates and the individual monomeric RNAi conjugates of Group 1 included modified nucleotides. The RNAi agent conjugates were synthesized according to phosphoramidite technology on solid phase in accordance with general procedures known in the art and commonly used in oligonucleotide synthesis, as set forth in Example 1 herein. For all of the RNAi agent conjugates, a targeting ligand was placed at the 5′ terminal end of the sense strand, as described in the following Table 4:

TABLE 4
RNAi agent conjugate duplexes from Example 4.

| Duplex | | SEQ | AS Gene |
|---|---|---|---|
| Name | Structure (5′→3′) | ID NO | Target |
| AD14219 | AS: cPrpusUfsgaguugaguUfcAfaGfugascsa | 1 | ANGPTL3 |
| | SS: NAG37sinvAbsugucacuuGfAfAfcucaacucaas(invAb) | 2 | |
| AD14220 | AS: cPrpusUfscsAfaAfgCfaCfuUfuAfuUfgAfsg | 3 | FXII |
| | SS: NAG37sinvAbscucaauAfAfAfgugcuuugaas(invAb) | 4 | |
| AD14216 | AS(1): cPrpusUfscsAfaAfgCfaCfuUfuAfuUfgAfsg | 5 | FXII |
| | AS(2): cPrpusUfsgaguugaguUfcAfaGfusgsa | 6 | ANGPTL3 |
| | SS: NAG37sucacuuGfAfAfcucaacucaas(invAb)- | 7 | |
| | Sp18csuscaauAfAfAfgugcuuugaas(invAb)s(C6-NH2) | | |
| AD14217 | AS(1): cPrpusUfscsAfaAfgCfaCfuUfuAfuUfgAfsg | 8 | FXII |
| | AS(2): cPrpusUfsgaguugaguUfcAfaGfusgsa | 9 | ANGPTL3 |
| | SS: NAG42sucacuuGfAfAfcucaacucaas(invAb)- | 10 | |
| | Sp18csuscaauAfAfAfgugcuuugaas(invAb)s(C6-NH2) | | |
| AD14218 | AS(1): cPrpusUfscsAfaAfgCfaCfuUfuAfuUfgAfsg | 11 | FXII |
| | AS(2): cPrpusUfsgaguugaguUfcAfaGfusgsa | 12 | ANGPTL3 |
| | SS: NAG52sucacuuGfAfAfcucaacucaas(invAb)- | 13 | |
| | Sp18csuscaauAfAfAfgugcuuugaas(invAb)s(C6-NH2) | | |

Table 4 Abbreviations: a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; s represents a phosphorothioate linkage; invAb represents an inverted abasic deoxyribose residue (see Table 2); sp18 represents the spacer 18 polyethylene glycol (PEG) linker as set forth in Table 2; C6-NH2 represents the aminolink linker or end cap as set forth in Table 2, NAG37s represents the N-Acetylgalactosamine trimer consisting of the structure represented as follows:

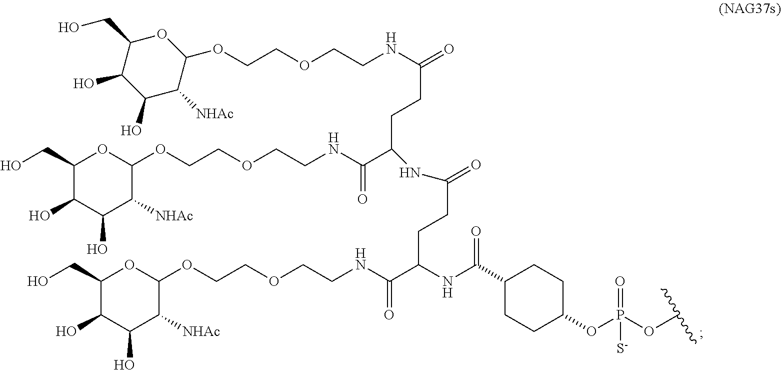 [SMILES: *OP(=O)([S-])O[C@H]1CC[C@@H](C(=O)NC(CCC(=O)NC(CCC(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)CC1]

NAG42s represents the metabolically stabilized NAG trimer having the structure represented in Table 2; and NAG52s represents the metabolically stabilized NAG trimer having the structure represented in Table 2.

The NAG37s structure was added to the sense strand as a phosphoramidite compound and was synthesized generally in accordance with International Patent Application Publication No. WO 2018/044350 to Arrowhead Pharmaceuticals, Inc., which is incorporated by reference in its entirety as if fully set forth herein. The NAG42s and NAG52s structures were also added to the sense strand in accordance with Examples 1, 2 and 3 herein.

In Table 4 above, AS refers to an antisense strand and SS refers to a sense strand. The individual nucleotides in a strand, while shown separated by commas in the Table above for convenience, are linked together by phosphodiester linkages unless an “s” is present, in which case the phosphorothioate linkage has replaced the phosphodiester linkage to link the nucleotides or non-nucleotide components of each respective strand. The antisense strands are then annealed to the respective sense strand. As used throughout herein for the multimeric RNAi agent conjugates disclosed, the “first” antisense strand or AS(1) in Table 4 above refers to the antisense strand that is located at the 3′-end of the sense strand multimeric RNAi conjugate complex. Each additional RNAi agent added to the multimeric conjugate (e.g., AS(2), AS(3), etc.), is be located further towards the 5′ end of the sense strand.

As noted above and discussed herein, the multimeric RNAi agent conjugate of Group 3 (AD14217) included the metabolically stabilized NAG of Formula II (and more specifically, Formula IIa), and the multimeric RNAi agent conjugate of Group 4 (AD14218) included the metabolically stabilized NAG of Formula I (and more specifically, Formula Ia). The multimeric RNAi agent conjugate of Group 2 (AD14216) and the monomeric RNAi agents of Group 1 (AD14219+AD14220) each included an N-Acetylgalactosamine targeting ligand that was not metabolically stabilized and having the structure set forth for NAG37s above. Each of the targeting ligands were linked to a respective RNAi agent through a phosphorothioate linkage.

Three (3) cynos were dosed in each Group (n=3). Serum samples were taken on days −14, −7, and day 1 (pre-dose). Monkeys were then administered according to the respective Groups as set forth in Table 5. Serum was then collected on days 8, 15, 22, 29, 36 43, 50, 57, 64, 71, 78, 98, 106, 120, 133, 148, and 162.

ANGPTL3 protein levels in serum were measured by ELISA assay (R&D Systems), according to the manufacturer's recommendations. FXII protein levels in serum were measured by ELISA assay (R&D Systems), according to the manufacturer's recommendations. The ANGPTL3 protein levels and FXII protein levels for each animal were normalized. For normalization, the level of ANGPTL3 protein or FXII protein, respectively, for each animal at a time point, was divided by the geometric mean of pre-treatment level of expression in that animal (in this case at days −14, −7 and 1 (pre-dose)) to determine the ratio of expression “normalized to pre-treatment.” Expression at a specific time point was then normalized to the saline control group by dividing the “normalized to pre-treatment” ratio for an individual animal by the mean “normalized to pretreatment” ratio of all mice in the saline control group. This resulted in expression for each time point normalized to that in the control group.

Data from the study set forth in this Example are shown in the following Tables 5 and 6:

TABLE 5
Average cynomolgus monkey ANGPTL3 Protein (cANGPTL3) Normalized to Pre-Treatment and Control from Example 4

| | Day 8 | | Day 15 | | Day 22 | | Day 29 | | Day 36 | |
|---|---|---|---|---|---|---|---|---|---|---|
| | Avg | Std Dev | Avg | Std Dev | Avg | Std Dev | Avg | Std Dev | Avg | Std Dev |
| Group ID | cANGPTL3 | (+/−) | cANGPTL3 | (+/−) | cANGPTL3 | (+/−) | cANGPTL3 | (+/−) | cANGPTL3 | (+/−) |
| Group 1 | 0.414 | 0.217 | 0.335 | 0.180 | 0.244 | 0.155 | 0.281 | 0.127 | 0.244 | 0.185 |
| (3.0 mg/kg AD14219 + | | | | | | | | | | |
| 3.0 mg/kg AD14220) | | | | | | | | | | |
| Group 2 | 0.442 | 0.130 | 0.362 | 0.120 | 0.339 | 0.170 | 0.403 | 0.175 | 0.369 | 0.207 |
| (6 mg/kg AD14216) | | | | | | | | | | |
| Group 3 | 0.361 | 0.019 | 0.288 | 0.030 | 0.194 | 0.033 | 0.261 | 0.073 | 0.246 | 0.067 |
| (6 mg/kg AD14217) | | | | | | | | | | |
| Group 4 | 0.252 | 0.034 | 0.238 | 0.049 | 0.137 | 0.038 | 0.161 | 0.059 | 0.186 | 0.042 |
| (6 mg/kg AD14218) | | | | | | | | | | |
| | Day 43 | | Day 50 | | Day 57 | | Day 64 | | Day 71 | |
| | Avg | Std Dev | Avg | Std Dev | Avg | Std Dev | Avg | Std Dev | Avg | Std Dev |
| | cANGPTL3 | (+/−) | cANGPTL3 | (+/−) | cANGPTL3 | (+/−) | cANGPTL3 | (+/−) | cANGPTL3 | (+/−) |
| Group 1 | 0.362 | 0.143 | 0.434 | 0.263 | 0.600 | 0.162 | 0.561 | 0.136 | 0.562 | 0.070 |
| (3.0 mg/kg AD14219 + | | | | | | | | | | |
| 3.0 mg/kg AD14220) | | | | | | | | | | |
| Group 2 | 0.394 | 0.159 | 0.560 | 0.259 | 0.622 | 0.301 | 0.590 | 0.270 | 0.641 | 0.220 |
| (6 mg/kg AD14216) | | | | | | | | | | |
| Group 3 | 0.272 | 0.078 | 0.327 | 0.075 | 0.456 | 0.121 | 0.548 | 0.164 | 0.521 | 0.129 |
| (6 mg/kg AD14217) | | | | | | | | | | |
| Group 4 | 0.164 | 0.042 | 0.212 | 0.084 | 0.245 | 0.043 | 0.352 | 0.171 | 0.328 | 0.129 |
| (6 mg/kg AD14218) | | | | | | | | | | |
| | Day 78 | | Day 98 | | Day 106 | | Day 120 | | Day 133 | |
| | Avg | Std Dev | Avg | Std Dev | Avg | Std Dev | Avg | Std Dev | Avg | Std Dev |
| Group ID | cANGPTL3 | (+/−) | cANGPTL3 | (+/−) | cANGPTL3 | (+/−) | cANGPTL3 | (+/−) | cANGPTL3 | (+/−) |
| Group 1 | 0.524 | 0.043 | 0.604 | 0.238 | 1.262 | 0.161 | 0.879 | 0.049 | 1.111 | 0.414 |
| (3.0 mg/kg AD14219 + | | | | | | | | | | |
| 3.0 mg/kg AD14220) | | | | | | | | | | |
| Group 2 | 0.603 | 0.160 | 0.666 | 0.233 | 0.944 | 0.244 | 1.128 | 0.388 | 1.129 | 0.302 |
| (6 mg/kg AD14216) | | | | | | | | | | |
| Group 3 | 0.505 | 0.099 | 0.490 | 0.059 | 0.770 | 0.009 | 0.943 | 0.161 | 0.892 | 0.053 |
| (6 mg/kg AD14217) | | | | | | | | | | |
| Group 4 | 0.354 | 0.059 | 0.425 | 0.049 | 0.565 | 0.058 | 0.637 | 0.052 | 0.672 | 0.087 |
| (6 mg/kg AD14218) | | | | | | | | | | |

TABLE 6
Average cynomolgus monkey FXII Protein (cFXII) Normalized
to Pre-Treatment and Control from Example 4

| | Day 8 | | Day 15 | | Day 22 | | Day 29 | | Day 36 | |
|---|---|---|---|---|---|---|---|---|---|---|
| | Avg | Std Dev | Avg | Std Dev | Avg | Std Dev | Avg | Std Dev | Avg | Std Dev |
| Group ID | cFXII | (+/−) | cFXII | (+/−) | cFXII | (+/−) | cFXII | (+/−) | cFXII | (+/−) |
| Group 1 | 0.493 | 0.167 | 0.250 | 0.083 | 0.295 | 0.136 | 0.207 | 0.118 | 0.231 | 0.083 |
| (3.0 mg/kg AD14219 + | | | | | | | | | | |
| 3.0 mg/kg AD14220) | | | | | | | | | | |
| Group 2 | 0.481 | 0.082 | 0.320 | 0.212 | 0.222 | 0.102 | 0.179 | 0.089 | 0.224 | 0.110 |
| (6 mg/kg AD14216) | | | | | | | | | | |
| Group 3 | 0.430 | 0.073 | 0.230 | 0.089 | 0.219 | 0.142 | 0.095 | 0.018 | 0.126 | 0.016 |
| (6 mg/kg AD14217) | | | | | | | | | | |
| Group 4 | 0.299 | 0.071 | 0.201 | 0.098 | 0.156 | 0.078 | 0.077 | 0.022 | 0.058 | 0.017 |
| (6 mg/kg AD14218) | | | | | | | | | | |
| | Day 43 | | Day 50 | | Day 57 | | Day 64 | | Day 71 | |
| | Avg | Std Dev | Avg | Std Dev | Avg | Std Dev | Avg | Std Dev | Avg | Std Dev |
| | cFXII | (+/−) | cFXII | (+/−) | cFXII | (+/−) | cFXII | (+/−) | cFXII | (+/−) |
| Group 1 | 0.339 | 0.184 | 0.155 | 0.051 | 0.242 | 0.048 | 0.211 | 0.033 | 0.390 | 0.079 |
| (3.0 mg/kg AD14219 + | | | | | | | | | | |
| 3.0 mg/kg AD14220) | | | | | | | | | | |
| Group 2 | 0.237 | 0.169 | 0.130 | 0.080 | 0.194 | 0.174 | 0.184 | 0.092 | 0.429 | 0.248 |
| (6 mg/kg AD14216) | | | | | | | | | | |
| Group 3 | 0.103 | 0.011 | 0.104 | 0.051 | 0.065 | 0.021 | 0.093 | 0.067 | 0.211 | 0.053 |
| (6 mg/kg AD14217) | | | | | | | | | | |
| Group 4 | 0.058 | 0.008 | 0.032 | 0.015 | 0.056 | 0.022 | 0.038 | 0.011 | 0.094 | 0.038 |
| (6 mg/kg AD14218) | | | | | | | | | | |
| | Day 78 | | Day 98 | | Day 106 | | Day 120 | | Day 133 | |
| | Avg | Std Dev | Avg | Std Dev | Avg | Std Dev | Avg | Std Dev | Avg | Std Dev |
| | cFXII | (+/−) | cFXII | (+/−) | cFXII | (+/−) | cFXII | (+/−) | cFXII | (+/−) |
| Group 1 | 0.610 | 0.088 | 0.750 | 0.008 | 0.653 | 0.063 | 0.694 | 0.059 | 0.895 | 0.155 |
| (3.0 mg/kg AD14219 + | | | | | | | | | | |
| 3.0 mg/kg AD14220) | | | | | | | | | | |
| Group 2 | 0.491 | 0.313 | 0.592 | 0.331 | 0.520 | 0.294 | 0.567 | 0.253 | 0.784 | 0.337 |
| (6 mg/kg AD14216) | | | | | | | | | | |
| Group 3 | 0.230 | 0.064 | 0.316 | 0.047 | 0.316 | 0.122 | 0.443 | 0.065 | 0.536 | 0.182 |
| (6 mg/kg AD14217) | | | | | | | | | | |
| Group 4 | 0.109 | 0.027 | 0.139 | 0.045 | 0.158 | 0.067 | 0.194 | 0.053 | 0.213 | 0.053 |
| (6 mg/kg AD14218) | | | | | | | | | | |

| | |
|---|---|
| Day 148 | Day 162 |

| | Avg | Std Dev | Avg | Std Dev |
|---|---|---|---|---|
| | cFXII | (+/−) | cFXII | (+/−) |
| Group 1 | 0.779 | 0.156 | 0.910 | 0.209 |
| (3.0 mg/kg AD14219 + | | | | |
| 3.0 mg/kg AD14220) | | | | |
| Group 2 | 0.589 | 0.176 | 0.728 | 0.322 |
| (6 mg/kg AD14216) | | | | |
| Group 3 | 0.567 | 0.122 | 0.468 | 0.335 |
| (6 mg/kg AD14217) | | | | |
| Group 4 | 0.263 | 0.091 | 0.285 | 0.112 |
| (6 mg/kg AD14218) | | | | |

This Example illustrates the utility of the delivery platforms of the present invention.

With respect to cyno ANGPTL3 protein levels, for example, at Day 22 (i.e., 3 weeks after dosing), the co-dosed monomer conjugates of Group 1 achieved approximately 76% cANGPTL3 knockdown (0.244) and Group 2 (using a dimer RNAi agent conjugate with a NAG targeting moiety) achieved approximately 66% cANGPTL3 knockdown (0.339), while the metabolically stabilized multimeric RNAi conjugates of Group 3 (dimer RNAi agent conjugate with metabolically stabilized NAG with beta anomeric linkage) and Group 4 (dimer RNAi agent conjugate with metabolically stabilized NAG with alpha anomeric linkage) showed approximately 81% knockdown (0.194) and 86% knockdown (0.137) of cANGPTL3, respectively. At Day 106 (i.e., more than 3 months after dosing), the co-dosed monomer conjugates of Group 1 and the dimer RNAi agent conjugate with NAG targeting moiety of Group 2 both essentially returned to baseline showing no meaningful knockdown of cANGPTL3. Conversely, at Day 106 the dimer RNAi agent conjugates with metabolically stabilized NAG targeting ligands of Group 3 (approximately 33% knockdown (0.777)) and Group 4 (approximately 45% knockdown (0.565)) still showed gene silencing activity.

With respect to cyno FXII protein levels, for example, at Day 22 (i.e., 3 weeks after dosing), the co-dosed monomer conjugates of Group 1 achieved approximately 71% cFXII knockdown (0.295); Group 2 (using a dimer RNAi agent conjugate with a NAG targeting moiety) and Group 3 (dimer RNAi agent conjugate with metabolically stabilized NAG with alpha anomeric linkage both attained approximately 78% cFXII knockdown), and Group 4 (dimer RNAi agent conjugate with metabolically stabilized NAG with beta anomeric linkage) showed approximately 85% knockdown (0.156). At Day 98 (i.e., more than 3 months after dosing), the co-dosed monomer conjugates of Group 1 returned to only showing 25% cFXII knockdown, and similarly the dimer RNAi agent conjugate with NAG targeting moiety of Group 2 showed only approximately 41% knockdown (0.592). Meanwhile, the dimer RNAi agent conjugates with metabolically stabilized NAG targeting ligands of Group 3 (approximately 69% knockdown (0.316)) and Group 4 (approximately 86% knockdown (0.139)) provided a substantially greater gene knockdown at Day 98, showing they have a greater duration of silencing activity in this study. Indeed, even at Day 162 (i.e., more than 5 months after dosing) the dimer RNAi agent conjugates with metabolically stabilized NAG Targeting Ligands of Group 4 (AD14218) continued to show cFXII inhibition of more than 70% (0.285).

While this particular example includes RNAi agents for the inhibition of ANGPTL3 and FXII, the same multimeric RNAi agent delivery platforms may be used to inhibit gene expression of other genes that are present in liver, including hepatocytes.

### Example 5. In Vivo Administration of APOC3-PCSK9 RNAi Agents in Cynomolgus Monkeys

APOC3-PCSK9 RNAi agents were tested in Cynomolgus monkeys for inhibition of APOC3 and PCSK9.

On Day 1, three (n=3) male Cynomolgus monkey test animals for each test group were dosed with APOC3-PCSK9 RNAi agents formulated in saline (at 6.0 mg/kg), via subcutaneous (SQ) injection with syringe and needle in the mid-scapular region, at 20.0 mL/kg dose volume.

Cynomolgus monkeys were acclimated for at least one (1) day. The animals were of 2 to 7 years. During the animals were not commingled for at least 24 hours after test article (RNAi agent) administration to allow for monitoring of any test article-related effects. The animals were fed with Certified Primate Diet $5048 (PMI, Inc.) and Greenfield city water provided ad libitum. Animals were maintained at a temperature of 20 to 26 degrees Centigrade, a relative humidity of 50+/−20%, and a 12-hour light/12-hour dark cycle.

The dosing regimen was in accordance with Table 7 below.

TABLE 7
Dosing for Cynomolgus animals of Example 5.

| Group | Dose | Dose | | # of Animals |
|---|---|---|---|---|
| ID | (RNAi Agent) | Concentration | Dosing Route | (n=) |
| 1 | 6.0 mg/kg | 20.0 mg/mL | Day 1 SQ | n = 3 |
| | AC003791 | | Injection | |
| 2 | 6.0 mg/kg | 20.0 mg/mL | Day 1 SQ | n = 3 |
| | AC005898 | | Injection | |

Each of the multimeric RNAi agent conjugates and the individual monomeric RNAi conjugates were synthesized according to phosphoramidite technology on solid phase in accordance with general procedures known in the art and commonly used in oligonucleotide synthesis. For the all of the RNAi agent conjugates, a targeting ligand was placed at the 5′ terminal end of the sense strand, as described in the following Table 8:

TABLE 8
RNAi agent conjugate duplexes from Example 5.

| Duplex | | SEQ ID | AS Gene |
|---|---|---|---|
| Name | Structure (5′→3′) | NO | Target |
| AC003791 | AS(1): cPrpasAfsguuacaaaaGfcAfaAfacsg | 14 | PCSK9 |
| | AS(2): cPrpusCfsasCfuGfagaauAfcUfgUfcCfsc | 15 | APOC3 |
| | SS: (NAG52)scguuuuGfcUfuUfuguaacuus(invAb)-Sp18- | 16 | |
| | gsggacaGfUfAfuucucaguias(invAb)s-C6-NH2 | | |
| AC005898 | AS(1): cPrpasCfsasAfaAfgCfaAfaAfcAfgGfuCfsc | 17 | PCSK9 |
| | AS(2): cPrpusCfsasCfuGfagaauAfcUfgUfcCfsc | 18 | APOC3 |
| | SS: (NAG52)sggaccuGfUfUfuugcuuuugus(invAb)-Sp18- | 19 | |
| | gsggacaGfUfAfuucucaguias(invAb)s-C6-NH2 | | |

Table 8 Abbreviations: a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; s represents a phosphorothioate linkage; invAb represents an inverted abasic deoxyribose residue (see Table 2); sp18 represents the spacer 18 polyethylene glycol (PEG) linker as set forth in Table 2; C6-NH2 represents the aminolink linker or end cap as set forth in Table 2, and NAG52s represents the metabolically stabilized NAG trimer having the structure represented in Table 2.

Before each SQ injection, the test animals were first sedated. Sedation was accomplished using Ketamine HCl (10 mg/kg) or Telazol (5-8 mg/kg), administered as an intramuscular (IM) injection and supplemented with Ketamine (5 mg/kg) as needed).

The test animals were dosed via subcutaneous SQ dose via syringe and needle in the scapular region (upper left, upper right, lower left, or lower right scapular region). The dose site was clipped free of hair at least one day prior to each dose administration. Individual doses of APOC3-PCSK9 RNAi agents were calculated based on the body weights recorded on each day of dosing. On each day of dose administration, the APOC3-PCSK9 RNAi agents were allowed to warm to ambient temperature at approximately room temperature for at least 30 minutes prior to the dose administration. Animals were fasted overnight prior to dosing.

Serum blood (approximately 5.0 mL) was collected on Day −6, Day 8, Day 15, Day 22, Day 29, Day 36, Day 43, Day 50, Day 57, and Day 63, prior to liver biopsy sample collections or dose administration (when applicable), and from any animals found in moribund condition or sacrificed at an unscheduled interval. The collection site was the femoral vein, with a saphenous vein as an alternative collection site.

The liver biopsies and serum collected from the test animals were used for analysis for APOC3 and PCSK9 expression and additional biological parameters. Liver biopsies were collected on Day −6, Day 15, Day 36, Day 50, and Day 64 (post-mortem).

Liver biopsies were collected as a sedated procedure. Animals were fasted overnight (at least 12 hours but less than 18 hours) prior to each liver biopsy collection. For each animal, collected liver biopsy samples were of approximately 100 mg each (80 to 120 mg).

The collected liver biopsies were analyzed for APOC3 and PCSK9 expression and additional biological parameters. Liver APOC3 and PCSK9 mRNA expression levels were quantified via qPCR, using cARL1 as endogenous control gene, normalized to Day-6 (pre-dose). The qPCR APOC3 and PCSK9 expression data is shown in the following Table 9 and Table 10.

TABLE 9
Liver APOC3 expression of Cynomolgus animals of Example 5.

| | Day −6 | | | Day 15 | | |
|---|---|---|---|---|---|---|
| | Rel. Exp. | Error | Error | Rel. Exp. | Error | Error |
| Group ID | APOC3 | Low | High | APOC3 | Low | High |
| 1. 6.0 mg/kg AC003791 | 1.000 | 0.210 | 0.266 | 0.408 | 0.131 | 0.194 |
| 2. 6.0 mg/kg AC005898 | 1.000 | 0.229 | 0.298 | 0.545 | 0.107 | 0.133 |
| | Day 36 | | | Day 50 | | |
| | Rel. Exp. | Error | Error | Rel. Exp. | Error | Error |
| Group ID | APOC3 | Low | High | APOC3 | Low | High |
| 1. 6.0 mg/kg AC003791 | 0.611 | 0.111 | 0.136 | 0.336 | 0.074 | 0.095 |
| 2. 6.0 mg/kg AC005898 | 0.929 | 0.220 | 0.288 | 0.491 | 0.161 | 0.239 |

| | Day 64 | | |
|---|---|---|---|
| | Rel. Exp. | Error | Error |
| Group ID | APOC3 | Low | High |
| 1. 6.0 mg/kg AC003791 | 0.500 | 0.054 | 0.061 |
| 2. 6.0 mg/kg AC005898 | 0.535 | 0.111 | 0.139 |

APOC3-PCSK9 RNAi agents achieved knockdown of APOC3 transcripts for a duration of at least 64 days, with subcutaneous SQ injection at 6.0 mg/kg on Day 1. Groups 1 and 2 achieved APOC3 knockdown. More specifically, AC003791 achieved approximately 66% inhibition (0.336) on Day 50 at 6.0 mg/kg. At Day 64, AC003791 achieved approximately 50% inhibition (0.500) at a single 6.0 mg/kg dose.

TABLE 10
Liver PCSK9 expression of Cynomolgus animals of Example 5.

| | Day −6 | | | Day 15 | | |
|---|---|---|---|---|---|---|
| | Rel. Exp. | Error | Error | Rel. Exp. | Error | Error |
| Group ID | PCSK9 | Low | High | PCSK9 | Low | High |
| 1. 6.0 mg/kg AC003791 | 1.000 | 0.255 | 0.343 | 0.692 | 0.133 | 0.165 |
| 2. 6.0 mg/kg AC005898 | 1.000 | 0.269 | 0.367 | 0.614 | 0.193 | 0.282 |
| | Day 36 | | | Day 50 | | |
| | Rel. Exp. | Error | Error | Rel. Exp. | Error | Error |
| Group ID | PCSK9 | Low | High | PCSK9 | Low | High |
| 1. 6.0 mg/kg AC003791 | 0.908 | 0.233 | 0.313 | 1.271 | 0.345 | 0.474 |
| 2. 6.0 mg/kg AC005898 | 0.520 | 0.228 | 0.407 | 0.528 | 0.155 | 0.219 |

| | Day 64 | | |
|---|---|---|---|
| | Rel. Exp. | Error | Error |
| Group ID | PCSK9 | Low | High |
| 1. 6.0 mg/kg AC003791 | 0.351 | 0.109 | 0.158 |
| 2. 6.0 mg/kg AC005898 | 0.457 | 0.085 | 0.104 |

APOC3-PCSK9 RNAi agents achieved knockdown of PCSK9 transcripts for a duration of at least 64 days, with single subcutaneous SQ injection at 6.0 mg/kg on Day 1. Groups 1 and 2 achieved PCSK9 knockdown. More specifically, AC003791 achieved approximately 64% inhibition (0.351) on Day 64 at single 6.0 mg/kg dose.

Serum PCSK9 was quantified via ELISA (R&D Systems, Cat. #DPC900) in accordance with manufacturer's instructions. The relative PCSK9 levels were normalized to pre-dose Day −6. The data is shown in the following Table 11.

TABLE 11
Serum PCSK9 expression of Cynomolgus animals of Example 5.

| | Day −6 | | Day 1 | | Day 8 | |
|---|---|---|---|---|---|---|
| | Rel. Exp. | Std. | Rel. Exp. | Std. | Rel. Exp. | Std. |
| Group ID | PCSK9 | Dev | PCSK9 | Dev | PCSK9 | Dev |
| 1. 6.0 mg/kg AC003791 | 0.925 | 0.299 | 1.161 | 0.376 | 0.682 | 0.393 |
| 2. 6.0 mg/kg AC005898 | 1.140 | 0.514 | 1.004 | 0.434 | 0.724 | 0.220 |
| | Day 15 | | Day 22 | | Day 29 | |
| | Rel. Exp. | Std. | Rel. Exp. | Std. | Rel. Exp. | Std. |
| Group ID | PCSK9 | Dev | PCSK9 | Dev | PCSK9 | Dev |
| 1. 6.0 mg/kg AC003791 | 0.415 | 0.061 | 0.339 | 0.211 | 0.353 | 0.130 |
| 2. 6.0 mg/kg AC005898 | 0.561 | 0.458 | 0.560 | 0.488 | 0.617 | 0.608 |
| | Day 36 | | Day 43 | | Day 50 | |
| | Rel. Exp. | Std. | Rel. Exp. | Std. | Rel. Exp. | Std. |
| Group ID | PCSK9 | Dev | PCSK9 | Dev | PCSK9 | Dev |
| 1. 6.0 mg/kg AC003791 | 0.323 | 0.036 | 0.375 | 0.115 | 0.759 | 0.926 |
| 2. 6.0 mg/kg AC005898 | 0.426 | 0.221 | 0.790 | 0.826 | 0.728 | 0.615 |

| | Day 57 | | Day 64 | |
|---|---|---|---|---|
| | Rel. Exp. | Std. | Rel. Exp. | Std. |
| Group ID | PCSK9 | Dev | PCSK9 | Dev |
| 1. 6.0 mg/kg AC003791 | 0.431 | 0.037 | 0.538 | 0.198 |
| 2. 6.0 mg/kg AC005898 | 0.762 | 0.645 | 0.928 | 0.638 |

APOC3-PCSK9 RNAi agents achieved knockdown of serum PCSK9 for a duration of at least 64 days, with single subcutaneous SQ injection at 6.0 mg/kg on Day 1. Groups 1 and 2 achieved PCSK9 knockdown. More specifically, AC003791 achieved approximately 67% inhibition (0.323) on Day 36 (nadir) at single 6.0 mg/kg dose. At Day 64, AC003791, with single 6.0 mg/kg dose, achieved approximately 46% inhibition (0.538).

Serum APOC3 was quantified via Roche Cobas® assay for APOC3 in accordance with manufacturer's instructions. The data is shown in the following Table 12.

TABLE 12
Serum APOC3 expression of Cynomolgus animals of Example 5.

| | Day −6 | | Day 1 | | Day 8 | |
|---|---|---|---|---|---|---|
| | APOC3 | Std. | APOC3 | Std. | APOC3 | Std. |
| Group ID | (mg/dL) | Dev | (mg/dL) | Dev | (mg/dL) | Dev |
| 1. 6.0 mg/kg AC003791 | 6.15 | 1.04 | 6.48 | 0.32 | 4.57 | 1.13 |
| 2. 6.0 mg/kg AC005898 | 5.84 | 0.79 | 6.10 | 0.34 | 4.61 | 1.23 |
| | Day 15 | | Day 22 | | Day 29 | |
| | APOC3 | Std. | APOC3 | Std. | APOC3 | Std. |
| Group ID | (mg/dL) | Dev | (mg/dL) | Dev | (mg/dL) | Dev |
| 1. 6.0 mg/kg AC003791 | 4.03 | 1.81 | 2.92 | 1.39 | 3.69 | 1.04 |
| 2. 6.0 mg/kg AC005898 | 3.59 | 0.45 | 3.50 | 0.81 | 4.16 | 0.85 |
| | Day 36 | | Day 43 | | Day 50 | |
| | APOC3 | Std. | APOC3 | Std. | APOC3 | Std. |
| Group ID | (mg/dL) | Dev | (mg/dL) | Dev | (mg/dL) | Dev |
| 1. 6.0 mg/kg AC003791 | 2.96 | 0.87 | 3.18 | 1.39 | 3.32 | 1.51 |
| 2. 6.0 mg/kg AC005898 | 3.59 | 0.78 | 4.07 | 1.04 | 3.96 | 1.38 |

| | Day 57 | | Day 64 | |
|---|---|---|---|---|
| | APOC3 | Std. | APOC3 | Std. |
| Group ID | (mg/dL) | Dev | (mg/dL) | Dev |
| 1. 6.0 mg/kg AC003791 | 4.56 | 1.37 | 4.65 | 0.99 |
| 2. 6.0 mg/kg AC005898 | 4.94 | 0.99 | 4.47 | 1.14 |

APOC3-PCSK9 RNAi agents achieved knockdown of serum APOC3 for a duration of at least 64 days, with single subcutaneous SQ injection at 6.0 mg/kg on Day 1. Groups 1 and 2 achieved APOC3 knockdown. More specifically, AC003791 achieved approximately 51% inhibition (2.96 mg/dL APOC3 on Day 36 relative to 6.15 mg/dL APOC3 on Day −6) on Day 36 (nadir) at single 6.0 mg/kg dose. Additionally, AC005898 achieved approximately 40% inhibition (3.50 mg/dL APOC3 on Day 22 relative to 5.84 mg/dL APOC3 on Day −6) on Day 22 (nadir) at single 6.0 mg/kg dose. At Day 64, AC003791, with single 6.0 mg/kg dose, achieved approximately 24% inhibition (4.65 mg/dL relative to 6.15 mg/dL on Day −6). At Day 64, AC005898, with single 6.0 mg/kg dose, achieved approximately 23% inhibition (4.47 mg/dL relative to 5.84 mg/dL on Day −6).

### Example 6. In Vivo Administration of Multimeric FXI-Gene X RNAi Agents in Cynomolgus Monkeys

FXII and a separate gene target produced in human hepatocytes (referred to as “Gene X”) multimeric RNAi agents were tested in Cynomolgus monkeys for inhibition of Factor XII (FXII) and Gene X. The AS(1) of each dimer used in this example are identical to AS(1) of AD14217 and AD14218, shown in Example 4, above. The AS(2) of each dimer used in this example are all the same across Groups 1, 2, and 3, and are complementary to a 19-nucleotide sequence of mRNA encoded by Gene X. The dimers used in this example included the same sense strand sequence as AD14217 and AD14218 as shown in Example 4, above, with the exceptions that the metabolically stabilized carbohydrate ligands are as indicated in Table 13, and that the sense strand portion that is complementary to ANGPTL3 AS(2) in AD14217 and AD 14218 are in this case are modified nucleotides complementary to the antisense sequence of Gene X AS(2).

On Day 1, three (n=3) male Cynomolgus monkeys for each test group were dosed with multimeric FXII-Gene X RNAi agents formulated in saline (at 6.0 mg/kg) via subcutaneous (SQ) injection with syringe and needle in the mid-scapular region, at 0.3 mL/kg dose volume.

The test animals were of Cynomolgus macaques (non-naïve) monkeys, male. The RNAi agent test articles were administered via subcutaneous (SQ) administration with a syringe and needle in the mid-scapular region.

The dosing regimen was in accordance with Table 13 below.

TABLE 13
Dosing for Cynomolgus monkeys of Example 6.

| Group | Dose | Dose | | # of Animals |
|---|---|---|---|---|
| ID | (RNAi Agent) | Volume | Dosing Route | (n=) |
| 1 | 6.0 mg/kg | 0.3 mL/kg | Day 1 SQ | n = 3 |
| | NAG52s | | Injection | |
| | conjugated | | | |
| | FXII-Gene X | | | |
| | dimer | | | |
| 2 | 6.0 mg/kg | 0.3 mL/kg | Day 1 SQ | n = 3 |
| | NAG55s | | Injection | |
| | conjugated | | | |
| | FXII-Gene X | | | |
| | dimer | | | |
| 3 | 6.0 mg/kg | 0.3 mL/kg | Day 1 SQ | n = 3 |
| | NAG1008s | | Injection | |
| | conjugated | | | |
| | FXII-Gene X | | | |
| | dimer | | | |

Each of the multimeric RNAi agent conjugates were synthesized according to phosphoramidite technology on solid phase in accordance with general procedures known in the art and commonly used in oligonucleotide synthesis.

The (NAG52)s, (NAG55)s, and (NAG1008)s structures were also added to the sense strand in accordance with Examples 1, 2 and 3 herein.

As noted above and discussed herein, the multimeric RNAi agent conjugate of Group 1 included the metabolically stabilized targeting ligand (NAG52)s (see table 1), the multimeric RNAi agent conjugate of Group 2 included the metabolically stabilized targeting ligand (NAG55)s (see table 1), and the multimeric RNAi agent conjugate of Group 3 included the metabolically stabilized targeting ligand (NAG1008)s (see table 1).

Before each SQ injection, the test animals were first sedated. Sedation was accomplished using Ketamine HCl (10 mg/kg), administered as an intramuscular (IM) injection.

Individual doses of multimeric FXII-Gene X RNAi agents were calculated based on the body weights recorded on each day of dosing. Animals were fasted overnight prior to dosing, at least 12 hours but less than 24 hours.

Serum blood (approximately 5.0 mL) was collected on Days −14, −7, 1 (pre-dose). 8, 15, 22, 29, 36, 43, 50, 57, 64, 71, 78, 85, 92, and 99, and from any animals found in moribund condition or sacrificed at an unscheduled interval. The collection site was the femoral vein, with a saphenous vein as an alternative collection site.

The collected serum samples were analyzed for FXII and Gene X expression and additional biological parameters. Serum FXII and Gene X protein levels were quantified via ELISA in accordance with manufacturer's instructions with relative expression normalized to the pre-dose of each test group. The quantified FXII and Gene X protein levels are shown in Table 14 and Table 15 below.

TABLE 14
Serum FXII levels of Cynomolgus monkeys of Example 6.

| | Day −14 | | Day −7 | | Day 1 | |
|---|---|---|---|---|---|---|
| | Rel. Exp. | Std. | Rel. Exp. | Std. | Rel. Exp. | Std. |
| Group ID | FXII | Dev. | FXII | Dev. | FXII | Dev. |
| 1. 6.0 mg/kg NAG52s | 0.956 | 0.058 | 1.073 | 0.092 | 0.981 | 0.081 |
| dimer | | | | | | |
| 2. 6.0 mg/kg NAG55s | 0.998 | 0.071 | 1.089 | 0.041 | 0.923 | 0.057 |
| dimer | | | | | | |
| 3. 6.0 mg/kg NAG1008s | 1.032 | 0.121 | 0.929 | 0.048 | 1.051 | 0.079 |
| dimer | | | | | | |
| | Day 8 | | Day 15 | | Day 22 | |
| | Rel. Exp. | Std. | Rel. Exp. | Std. | Rel. Exp. | Std. |
| Group ID | FXII | Dev. | FXII | Dev. | FXII | Dev. |
| 1. 6.0 mg/kg NAG52s | 0.601 | 0.116 | 0.339 | 0.108 | 0.266 | 0.103 |
| dimer | | | | | | |
| 2. 6.0 mg/kg NAG55s | 0.510 | 0.014 | 0.306 | 0.025 | 0.284 | 0.042 |
| dimer | | | | | | |
| 3. 6.0 mg/kg NAG1008s | 0.644 | 0.017 | 0.467 | 0.076 | 0.448 | 0.071 |
| dimer | | | | | | |
| | Day 29 | | Day 36 | | Day 43 | |
| | Rel. Exp. | Std. | Rel. Exp. | Std. | Rel. Exp. | Std. |
| Group ID | FXII | Dev. | FXII | Dev. | FXII | Dev. |
| 1. 6.0 mg/kg NAG52s | 0.252 | 0.095 | 0.272 | 0.117 | 0.311 | 0.150 |
| dimer | | | | | | |
| 2. 6.0 mg/kg NAG55s | 0.240 | 0.051 | 0.238 | 0.035 | 0.275 | 0.014 |
| dimer | | | | | | |
| 3. 6.0 mg/kg NAG1008s | 0.460 | 0.073 | 0.458 | 0.090 | 0.527 | 0.150 |
| dimer | | | | | | |
| | Day 50 | | Day 57 | | Day 64 | |
| | Rel. Exp. | Std. | Rel. Exp. | Std. | Rel. Exp. | Std. |
| Group ID | FXII | Dev. | FXII | Dev. | FXII | Dev. |
| 1. 6.0 mg/kg NAG52s | 0.381 | 0.187 | 0.356 | 0.218 | 0.315 | 0.192 |
| dimer | | | | | | |
| 2. 6.0 mg/kg NAG55s | 0.348 | 0.029 | 0.413 | 0.080 | 0.307 | 0.022 |
| dimer | | | | | | |
| 3. 6.0 mg/kg NAG1008s | 0.625 | 0.094 | 0.695 | 0.210 | 0.561 | 0.057 |
| dimer | | | | | | |
| | Day 71 | | Day 78 | | Day 85 | |
| | Rel. Exp. | Std. | Rel. Exp. | Std. | Rel. Exp. | Std. |
| Group ID | FXII | Dev. | FXII | Dev. | FXII | Dev. |
| 1. 6.0 mg/kg NAG52s | 0.345 | 0.231 | 0.374 | 0.240 | 0.414 | 0.282 |
| dimer | | | | | | |
| 2. 6.0 mg/kg NAG55s | 0.358 | 0.037 | 0.436 | 0.029 | 0.462 | 0.024 |
| dimer | | | | | | |
| 3. 6.0 mg/kg NAG1008s | 0.564 | 0.117 | 0.685 | 0.149 | 0.749 | 0.231 |
| dimer | | | | | | |

| | Day 92 | | Day 99 | |
|---|---|---|---|---|
| | Rel. Exp. | Std. | Rel. Exp. | Std. |
| Group ID | FXII | Dev. | FXII | Dev. |
| 1. 6.0 mg/kg NAG52s | 0.360 | 0.226 | 0.363 | 0.250 |
| dimer | | | | |
| 2. 6.0 mg/kg NAG55s | 0.511 | 0.047 | 0.503 | 0.051 |
| dimer | | | | |
| 3. 6.0 mg/kg NAG1008s | 0.739 | 0.168 | 0.726 | 0.152 |
| dimer | | | | |

FXII-Gene X multimeric RNAi agents achieved FXII inhibition out to at least Day 99, with single subcutaneous SQ injection at 6.0 mg/kg on Day 1. At nadir, a single 6.0 mg/kg dose NAG55s conjugated dimer achieved ˜76 FXII inhibition (0.238) on Day 36. At Day 99, a single 6.0 mg/kg dose NAG52s conjugated dimer achieved ˜63% FXII inhibition (0.363).

TABLE 15
Serum Gene X levels of Cynomolgus monkeys of Example 6.

| | Day −14 | | Day −7 | | Day 1 | |
|---|---|---|---|---|---|---|
| | Rel. Exp. | Std. | Rel. Exp. | Std. | Rel. Exp. | Std. |
| Group ID | Gene X | Dev. | Gene X | Dev. | Gene X | Dev. |
| 1. 6.0 mg/kg NAG52s | 0.764 | 0.049 | 1.201 | 0.025 | 1.094 | 0.075 |
| dimer | | | | | | |
| 2. 6.0 mg/kg NAG55s | 0.860 | 0.078 | 1.128 | 0.065 | 1.041 | 0.131 |
| dimer | | | | | | |
| 3. 6.0 mg/kg NAG1008s | 1.081 | 0.247 | 1.030 | 0.054 | 0.928 | 0.193 |
| dimer | | | | | | |
| | Day 8 | | Day 15 | | Day 22 | |
| | Rel. Exp. | Std. | Rel. Exp. | Std. | Rel. Exp. | Std. |
| Group ID | Gene X | Dev. | Gene X | Dev. | Gene X | Dev. |
| 1. 6.0 mg/kg NAG52s | 0.332 | 0.088 | 0.186 | 0.063 | 0.129 | 0.058 |
| dimer | | | | | | |
| 2. 6.0 mg/kg NAG55s | 0.414 | 0.053 | 0.237 | 0.091 | 0.223 | 0.122 |
| dimer | | | | | | |
| 3. 6.0 mg/kg NAG1008s | 0.509 | 0.070 | 0.267 | 0.084 | 0.294 | 0.088 |
| dimer | | | | | | |
| | Day 29 | | Day 36 | | Day 43 | |
| | Rel. Exp. | Std. | Rel. Exp. | Std. | Rel. Exp. | Std. |
| Group ID | Gene X | Dev. | Gene X | Dev. | Gene X | Dev. |
| 1. 6.0 mg/kg NAG52s | 0.181 | 0.100 | 0.202 | 0.109 | 0.252 | 0.138 |
| dimer | | | | | | |
| 2. 6.0 mg/kg NAG55s | 0.259 | 0.097 | 0.226 | 0.055 | 0.302 | 0.068 |
| dimer | | | | | | |
| 3. 6.0 mg/kg NAG1008s | 0.349 | 0.119 | 0.266 | 0.103 | 0.460 | 0.195 |
| dimer | | | | | | |
| | Day 50 | | Day 57 | | Day 64 | |
| | Rel. Exp. | Std. | Rel. Exp. | Std. | Rel. Exp. | Std. |
| Group ID | Gene X | Dev. | Gene X | Dev. | Gene X | Dev. |
| 1. 6.0 mg/kg NAG52s | 0.313 | 0.189 | 0.324 | 0.216 | 0.236 | 0.190 |
| dimer | | | | | | |
| 2. 6.0 mg/kg NAG55s | 0.355 | 0.072 | 0.405 | 0.082 | 0.286 | 0.045 |
| dimer | | | | | | |
| 3. 6.0 mg/kg NAG1008s | 0.514 | 0.205 | 0.628 | 0.211 | 0.503 | 0.061 |
| dimer | | | | | | |
| | Day 71 | | Day 78 | | Day 85 | |
| | Rel. Exp. | Std. | Rel. Exp. | Std. | Rel. Exp. | Std. |
| Group ID | Gene X | Dev. | Gene X | Dev. | Gene X | Dev. |
| 1. 6.0 mg/kg NAG52s | 0.266 | 0.182 | 0.308 | 0.216 | 0.344 | 0.261 |
| dimer | | | | | | |
| 2. 6.0 mg/kg NAG55s | 0.321 | 0.058 | 0.365 | 0.058 | 0.411 | 0.039 |
| dimer | | | | | | |
| 3. 6.0 mg/kg NAG1008s | 0.475 | 0.101 | 0.456 | 0.105 | 0.459 | 0.103 |
| dimer | | | | | | |

| | Day 92 | | Day 99 | |
|---|---|---|---|---|
| | Rel. Exp. | Std. | Rel. Exp. | Std. |
| Group ID | Gene X | Dev. | Gene X | Dev. |
| 1. 6.0 mg/kg NAG52s | 0.267 | 0.191 | 0.284 | 0.208 |
| dimer | | | | |
| 2. 6.0 mg/kg NAG55s | 0.397 | 0.041 | 0.469 | 0.069 |
| dimer | | | | |
| 3. 6.0 mg/kg NAG1008s | 0.443 | 0.141 | 0.466 | 0.152 |
| dimer | | | | |

FXII-Gene X multimeric RNAi agents achieved Gene X inhibition out to at least Day 99, with single subcutaneous SQ injection at 6.0 mg/kg on Day 1. At nadir, a single 6.0 mg/kg dose NAG52s conjugated dimer achieved ˜87% inhibition (0.129) of Gene X on Day 22. At Day 99, a single 6.0 mg/kg dose NAG52s conjugated dimer achieved ˜71% inhibition of Gene X (0.284).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound for inhibiting expression of one or more genes, comprising:
a. an oligonucleotide comprising a strand 12-49 nucleotides in length; and
b. a metabolically stabilized carbohydrate ligand;
wherein the oligonucleotide and the metabolically stabilized carbohydrate ligand are covalently bound by a phosphorothioate linkage, a phosphorodithioate linkage, or another linkage that is more metabolically stable than a phosphodiester linkage, or a pharmaceutically acceptable salt thereof.

2. The compound of claim 1 or a pharmaceutically acceptable salt thereof, wherein the metabolically stabilized carbohydrate ligand is of the formula: 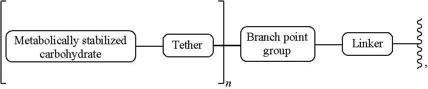
wherein:
each instance of the metabolically stabilized carbohydrate is independently a chemically modified carbohydrate moiety;
each instance of the tether is independently of the formula: 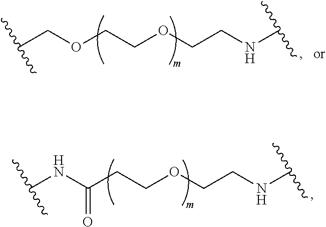
wherein m is an integer selected from 1 to 20;
the branch point group is a structure selected from the group consisting of: 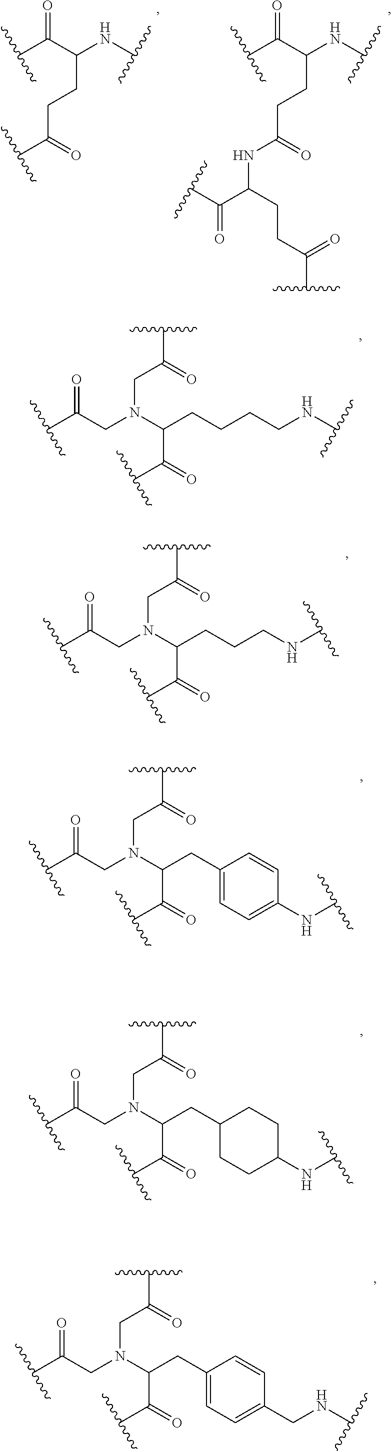 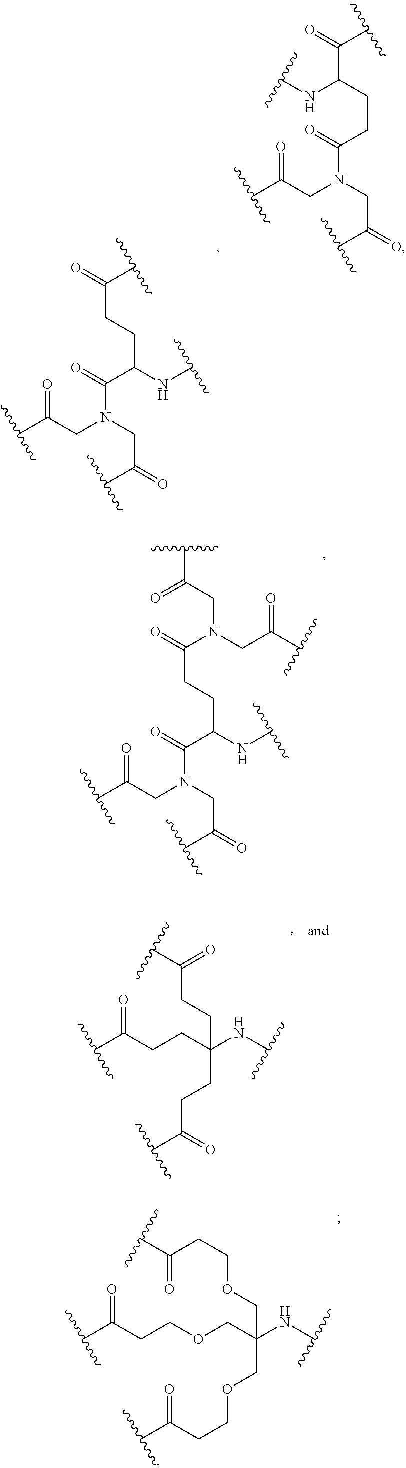 [SMILES: *NC(CCC(=O)N(CC(*)=O)CC(*)=O)C(*)=O ; *NC(CCC(*)=O)C(=O)N(CC(*)=O)CC(*)=O ; *NC(CCC(=O)N(CC(*)=O)CC(*)=O)C(=O)N(CC(*)=O)CC(*)=O ; *NC(CCC(*)=O)(CCC(*)=O)CCC(*)=O ; *NC(COCCC(*)=O)(COCCC(*)=O)COCCC(*)=O]
the linker is a structure selected from the group consisting of: 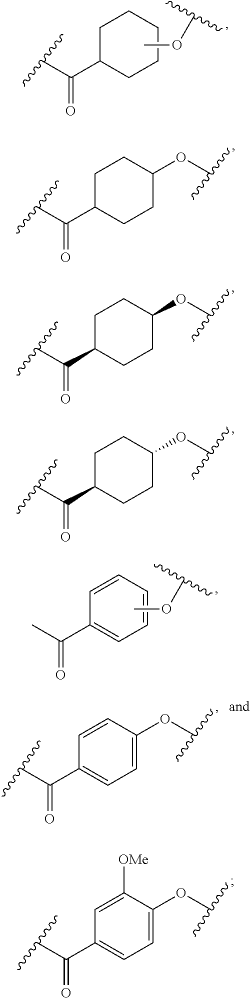
n is an integer from 1 to 4, as valency permits; and [symbol]
indicates the attachment point to the remainder of the compound.

3. The compound of claim 2, wherein the tether is of the formula:

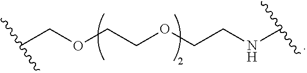

4. The compound of claim 2, wherein the tether is of the formula:

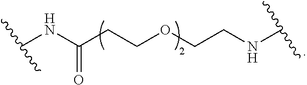

5. The compound of claim 2, wherein the branch point group is of the formula:

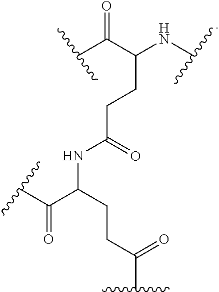 [SMILES: *NC(CCC(=O)NC(CCC(*)=O)C(*)=O)C(*)=O]

6. (canceled)

7. The compound of claim 2, wherein the branch point group is of the formula:

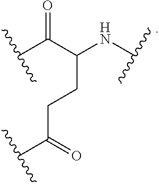 [SMILES: *NC(CCC(*)=O)C(*)=O]

8. (canceled)

9. The compound of claim 1, wherein the metabolically stabilized carbohydrate ligand comprises metabolically stabilized N-Acetylgalactosamine.

10. The compound of any one of claims 1-9, wherein the metabolically stabilized carbohydrate is of the formula: 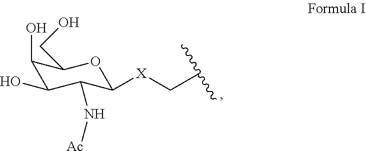
wherein X is CH₂ or S, and [symbol] indicates the point of connection to the remainder of the compound.

11. The compound of any one of claims 1-9, wherein the metabolically stabilized carbohydrate is of the formula: 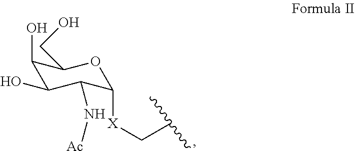
wherein X is CH₂ or S, and [symbol] indicates the point of connection to the remainder of the compound.

12. The compound of claim 10 or 11, wherein X is CH₂.

13. The compound of claim 10 or 11, wherein X is S.

14. The compound of any one of claims 1-6, or 9-13, wherein the metabolically stabilized carbohydrate ligand comprises three metabolically stabilized N-Acetylgalactosamine moieties.

15. The compound of any one of claims 2-6, or 9-13, wherein n is 3.

16. The compound of any one of claims 2-15, wherein the linker is of the formula:

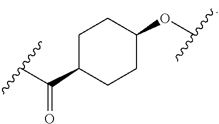 [SMILES: *O[C@H]1CC[C@@H](C(*)=O)CC1]

17. The compound of any one of claims 1-16, wherein the metabolically stabilized carbohydrate ligand comprises a structure selected from the group consisting of: 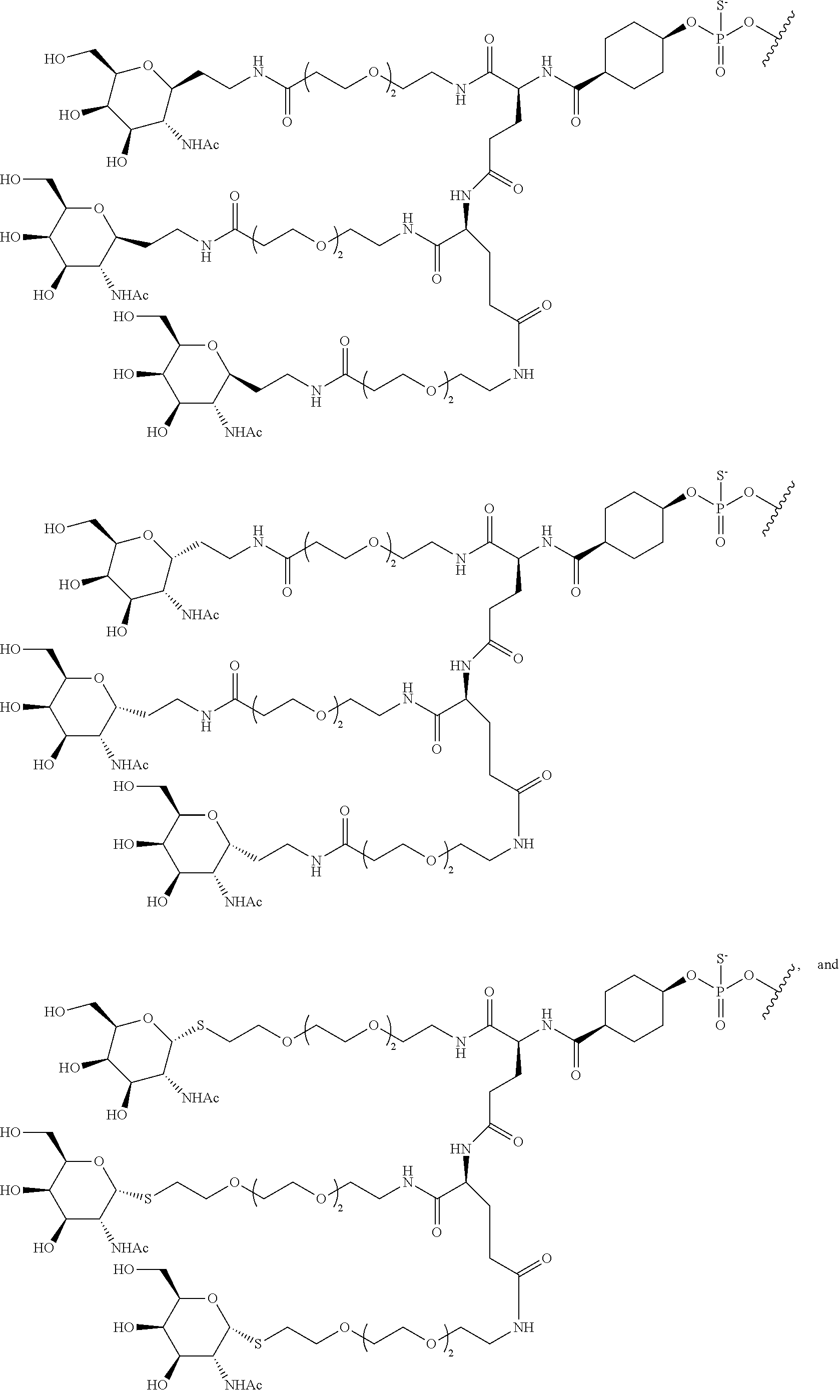 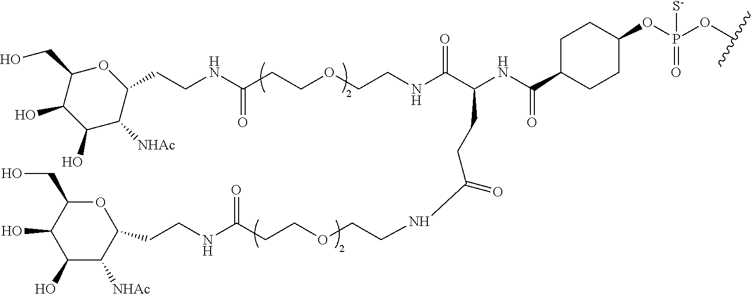
wherein [symbol] indicates the point of connection to the remainder of the compound.

18-22. (canceled)

23. The compound of any one of claims 1-22, wherein the metabolically stabilized carbohydrate ligand is conjugated to the 3′ terminus of the sense strand.

24. The compound of any one of claims 1-23, wherein the metabolically stabilized carbohydrate ligand is conjugated to the 5′ terminus of the sense strand.

25-39. (canceled)

40. A pharmaceutical composition comprising a compound of any one of claims 1-39 and a pharmaceutically acceptable excipient.

41. A method of inhibiting expression of a gene, comprising administering to a subject in need thereof a compound of any one of claims 1-39.

42. A method of inhibiting expression of a gene, comprising administering to a subject in need thereof the pharmaceutical composition of claim 40.
